SEQUENCE LISTING (3 sequences)

SEQ ID NO: 1 - DNA, 22 bp, Artificial
  Synthetic Forward Primer
caacatgaatgccattttccaa

SEQ ID NO: 2 - DNA, 21 bp, Artificial
  Synthetic Reverse Primer
ataatcccctgaacccaagga

SEQ ID NO: 3 - DNA, 25 bp, Artificial
  Synthetic Probe
taaagccatgccctctgcaggaaca

(19) United States
(12) Patent Application Publication
(10) Pub. No.: US 2010/0273816 A1
(43) Pub. Date: Oct. 28, 2010

(54) Quinazoline Compounds

(76) Inventors: Ronald Charles Bernotas, Royersford, PA (US); John William Ullrich, Downingtown, PA (US); Jeremy Mark Travins, Downingtown, PA (US); Jay Edward Wrobel, Lawrenceville, NJ (US); Rayomand Jal Unwalla, Eagleville, PA (US)

Correspondence Address: WYETH LLC;PATENT LAW GROUP, 5 GIRALDA FARMS, MADISON, NJ, 07940, US

(21) Appl. No.: 12/600,737

(22) PCT Filed: May 15, 2008
(86) PCT No.: PCT/US08/63685

§ 371 Date: May 28, 2010

Related U.S. Application Data
(60) Provisional application No. 60/938,801, filed on May 18, 2007.

(51) Int. Cl. A61K 31/517; C07D 239/74; A61P 9/00; A61P 25/28; A61P 3/10; A61P 29/00; A61P 1/16; A61P 19/02; A61P 9/10; A61P 19/04; A61P 17/00; A61P 3/04

(52) U.S. Cl. 514/266.1; 544/283

(57) ABSTRACT

Disclosed are quinazoline-based modulators of Liver X receptors (LXRs) and related methods. The modulators include compounds of formula (I):

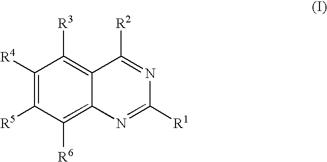

in which R¹, R², R³, R⁴, R⁵, R⁶, R⁷, R⁸, R⁹, R¹⁰, R¹¹, R¹², R¹³, R¹⁴, R¹⁵, W, W¹, W², W³, A, R^{a}, R^{a′}, R^{b}, R^{b′}, R^{c}, R^{d}, R^{d′}, R^{e}, R^{f}, R^{g}, R^{h}, Rⁱ R^{j}, R^{k}, R^{m}, Rⁿ, R^{o}, R^{p}, R^{q}, and n, can be, independently, as defined anywhere herein. In general, these compounds can be used for treating or preventing one or more diseases, disorders, conditions or symptoms mediated by LXRs.

### CROSS REFERENCE TO RELATED APPLICATIONS

This application claims the benefit of U.S. Provisional Application No. 60/938,801, filed on May 18, 2007, which is incorporated herein by reference in its entirety.

### TECHNICAL FIELD

This invention relates generally to quinazoline-based modulators of Liver X receptors (LXRs) and related methods.

### BACKGROUND

Atherosclerosis is among the leading causes of death in developed countries. Some of the independent risk factors associated with atherosclerosis include the presence of relatively high levels of serum LDL cholesterol and relatively low levels of serum HDL cholesterol in affected patients. As such, some anti-atherosclerotic therapy regimens include the administration of agents (e.g., statins) to reduce elevated serum LDL cholesterol levels.

Agents that increase patient HDL cholesterol levels can also be useful in anti-atherosclerotic therapy regimens. HDL cholesterol is believed to play a major role in the transport of cholesterol from peripheral tissues to the liver for metabolism and excretion (this process is sometimes referred to as “reverse cholesterol transport”). ABCA1 is a transporter gene involved in HDL production and reverse cholesterol transport. Upregulation of ABCA1 can therefore result in increased reverse cholesterol transport as well as inhibition of cholesterol absorption in the gut. In addition, HDL is also believed to inhibit the oxidation of LDL cholesterol, reduce the inflammatory response of endothelial cells, inhibit the coagulation pathway, and promote the availability of nitric oxide.

Liver X receptors (LXRs), originally identified in the liver as orphan receptors, are members of the nuclear hormone receptor super family and are believed to be involved in the regulation of cholesterol and lipid metabolism. LXRs are ligand-activated transcription factors and bind to DNA as obligate heterodimers with retinoid X receptors. While LXRα is generally found in tissues such as liver, kidney, adipose tissue, intestine and macrophages, LXRβ displays a ubiquitous tissue distribution pattern. Activation of LXRs by oxysterols (endogenous ligands) in macrophages results in the expression of several genes involved in lipid metabolism and reverse cholesterol transport including the aforementioned ABCA1; ABCG1; and ApoE.

Studies have been conducted in LXRα knock-out (k/o), LXRβ k/o and double k/o mice to determine the physiological role of LXRs in lipid homeostasis and atherosclerosis. The data from these studies suggested that in double k/o mice on normal chow diet, increased cholesterol accumulation was observed in macrophages (foam cells) of the spleen, lung and arterial wall. The increased cholesterol accumulation was believed to be associated with the presence of reduced serum HDL cholesterol and increased LDL cholesterol, even though the total cholesterol levels in the mice were about normal. While LXRα k/o mice did not appear to show significant changes in hepatic gene expression, LXRβ k/o mice showed 58% decrease in hepatic ABCA1 expression and 208% increase in SREBP1c expression suggesting that LXRβ may be involved in the regulation of liver SREBP1c expression.

Data obtained from studies employing two different atherosclerotic mouse models (ApoE k/o and LDLR k/o) suggest that agonists of LXRα or β can be relatively effective in upregulating ABCA1 expression in macrophages. For example, inhibition of atherosclerotic lesions could be observed when ApoE k/o and LDLR k/o mice were treated with LXRα or β agonists for 12 weeks. The tested agonists were observed to have variable effects on serum cholesterol and lipoprotein levels and appeared to cause a relatively significant increase in serum HDL cholesterol and triglyceride levels. These in vivo data were found to be consistent with in vitro data obtained for the same agonists in macrophages.

In addition to the lipid and triglyceride effects described above, it is also believed that activation of LXRs results in the inhibition of inflammation and proinflammatory gene expression. This hypothesis is based on data obtained from studies employing three different models of inflammation (LPS-induced sepsis, acute contact dermatitis of the ear and chronic atherosclerotic inflammation of the artery wall). These data suggest that LXR modulators can mediate both the removal of cholesterol from the macrophages and the inhibition of vascular inflammation.

### SUMMARY

This invention relates generally to quinazoline-based modulators of Liver X receptors (LXRs) and related methods.

In one aspect, this invention features a compound having formula (I):

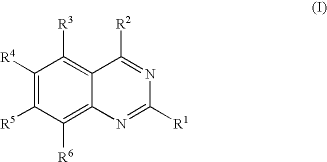

in which:

R¹ is:

(i) hydrogen; or

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{b}; or

(iv) C₃-C₂₀ cycloalkyl, C₃-C₂₀ cycloalkenyl, heterocyclyl including 3-20 atoms, heterocycloalkenyl including 3-20 atoms, C₇-C₂₀ aralkyl, or heteroaralkyl including 6-20 atoms, each of which is optionally substituted with from 1-10 R^{c}; or

(v) C₆-C₁₈ aryl or heteroaryl including 5-16 atoms, each of which is optionally substituted with from 1-10 R^{d};

R² is C₆-C₁₈ aryl or heteroaryl including 5-16 atoms, in which the aryl or heteroaryl is:

(i) substituted with from 1-5 R⁷, and

(ii) optionally substituted with from 1-4 R^{e}; wherein:

R⁷ is WA, wherein:

W at each occurrence is, independently, a bond; —O—; —NR⁸— wherein R⁸ is hydrogen, C₁-C₆ alkyl, C₃-C₇ cycloalkyl, or C₆-C₁₀ aryl or heteroaryl including 5-10 atoms in which the aryl or heteroaryl group is optionally substituted with from 1-5 R^{d}; C₁₋₆ alkylene, C₂₋₆ alkenylene, or C₂₋₆ alkynylene, each of which is optionally substituted with from 1-5 R^{f}; —W¹(C₁₋₆ alkylene)-; or —(C₁₋₆ alkylene)W¹—;

W¹ at each occurrence is, independently, —O— or —NR⁸—; and

A at each occurrence is, independently, C₆-C₁₈ aryl or heteroaryl including 5-16 atoms, each of which is:

(i) substituted with from 1-5 R⁹, and

(ii) optionally further substituted with from 1-10 R^{g};

R⁹ at each occurrence is, independently:

(i) —W²—S(O)ₙR¹⁰ or —W²—S(O)ₙNR¹¹R¹²; or

(ii) —W²—C(O)OR¹³; or

(iii) —W²—C(O)NR¹¹R¹²; or

(iv) —W²—CN; or

(v) C₁-C₁₂ alkyl or C₁-C₁₂ haloalkyl, each of which is:
(a) substituted with from 1-3 R^{h}, and
(b) optionally further substituted with from 1-5 R^{a};

or

(vi) C₇-C₂₀ aralkyl or heteroaralkyl including 6-20 atoms, each of which is:
(a) substituted with from 1-3 R^{h}, and
(b) optionally further substituted with from 1-5 substituents independently selected from R^{a}; C₁-C₆ alkyl, which is optionally substituted with from 1-3 R^{a}; C₁-C₆ haloalkyl; C₆-C₁₀ aryl, which is optionally substituted with from 1-10 R^{d}; halo; C₂-C₆ alkenyl; or C₂-C₆ alkynyl;

or

(vii) —NR¹⁴R¹⁵;

wherein:

W² at each occurrence is, independently, a bond; C₁₋₆ alkylene optionally substituted with from 1-3 R^{f}; C₂₋₆ alkenylene; C₂₋₆ alkynylene; C₃₋₆ cycloalkylene; —O(C₁₋₆ alkylene)-, or —NR⁸(C₁₋₆ alkylene)-;

n at each occurrence is, independently, 1 or 2;

R¹⁰ at each occurrence is, independently:

(i) C₁-C₂₀ alkyl or C₁-C₂₀ haloalkyl, each of which is optionally substituted with from 1-10 R^{a}; or

(ii) C₂-C₂₀ alkenyl or C₂-C₂₀ alkynyl, each of which is optionally substituted with from 1-10 R^{b}; or

(iii) C₃-C₂₀ cycloalkyl, C₃-C₂₀ cycloalkenyl, C₇-C₂₀ aralkyl, or heteroaralkyl including 6-20 atoms, each of which is optionally substituted with from 1-10 R^{c}; or

(iv) C₆-C₁₈ aryl or heteroaryl including 5-16 atoms, each of which is optionally substituted with from 1-10 R^{d};

R¹¹ and R¹² are each, independently, hydrogen; R¹⁰; or heterocyclyl including 3-20 atoms or a heterocycloalkenyl including 3-20 atoms, each of which is optionally substituted with from 1-5 R^{c}; or

R¹¹ and R¹² together with the nitrogen atom to which they are attached form a heterocyclyl including 3-20 atoms or a heterocycloalkenyl including 3-20 atoms, each of which is optionally substituted with from 1-5 R^{c};

R¹³ at each occurrence is, independently, hydrogen or R¹⁰;

at each occurrence of —NR¹⁴R¹⁵, one of R¹⁴ and R¹⁵ is hydrogen or C₁-C₃ alkyl; and the other of R¹⁴ and R¹⁵ is:

(i) —S(O)ₙR¹⁰; or

(ii) —C(O)OR¹³; or

(iii) —C(O)NR¹¹R¹²; or

(iv) —CN; or

(v) C₁-C₁₂ alkyl or C₁-C₁₂ haloalkyl, each of which is:
(a) substituted with from 1-3 R^{h}, and
(b) optionally further substituted with from 1-5 R^{a};

or

(vi) C₇-C₂₀ aralkyl or heteroaralkyl including 6-20 atoms, each of which is:
(a) substituted with from 1-3 R^{h}, and
(b) optionally further substituted with from 1-5 substituents independently selected from R^{a}; C₁-C₆ alkyl, which is optionally substituted with from 1-3 R^{a}; C₁-C₆ haloalkyl; C₆-C₁₀ aryl, which is optionally substituted with from 1-10 R^{d}; halo; C₂-C₆ alkenyl; or C₂-C₆ alkynyl;

each of R³, R⁴, and R⁵ is, independently:

(i) hydrogen; or

(ii) halo; or

(iii) C₁-C₆ alkyl or C₁-C₆ haloalkyl, each of which is optionally substituted with from 1-3 R^{a}; or

(iv) NRⁱR^{j}, wherein each of Rⁱ and R^{j} is, independently, hydrogen or C₁-C₃ alkyl; nitro; azido; hydroxy; C₁-C₆ alkoxy; C₁-C₆ haloalkoxy; C₆-C₁₀ aryloxy or heteroaryloxy including 5-10 atoms, each of which is optionally substituted with from 1-5 R^{d}; C₇-C₁₀ aralkoxy, heteroaralkoxy including 6-10 atoms, C₃-C₆ cycloalkoxy, C₃-C₆ cycloalkenyloxy, heterocyclyloxy including 3-6 atoms, or heterocycloalkenyloxy including 3-6 atoms, each of which is optionally substituted with from 1-5 R^{c}; mercapto; C₁-C₆ thioalkoxy; C₁-C₆ thiohaloalkoxy; C₆-C₁₀ thioaryloxy or thioheteroaryloxy including 5-10 atoms, each of which is optionally substituted with from 1-10 R^{d}; C₇-C₁₀ thioaralkoxy, thioheteroaralkoxy including 6-10 atoms, C₃-C₆ thiocycloalkoxy, C₃-C₆ thiocycloalkenyloxy, thioheterocyclyloxy including 3-6 atoms, or thioheterocycloalkenyloxy including 3-6 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or

(v) C₂-C₆ alkenyl or C₂-C₆ alkynyl, each of which is optionally substituted with from 1-10 R^{b}; or

(vi) C₇-C₁₀ aralkyl, heteroaralkyl including 6-10 atoms, C₃-C₆ cycloalkyl, C₃-C₆ cycloalkenyl, heterocyclyl including 3-6 atoms, or heterocycloalkenyl including 3-6 atoms, each of which is optionally substituted with from 1-3 R^{c}; or

(vii) C₆-C₁₀ aryl or heteroaryl including 5-10 atoms, each of which is optionally substituted with from 1-10 R^{d};

R⁶ is:

(i) halo; or

(ii) C₁-C₆ alkyl or C₁-C₆ haloalkyl, each of which is optionally substituted with from 1-3 R^{a}; or

(iii) nitro; azido; C₁-C₆ alkoxy; C₁-C₆ haloalkoxy; C₆-C₁₀ aryloxy or heteroaryloxy including 5-10 atoms, each of which is optionally substituted with from 1-5 R^{d}; C₇-C₁₀ aralkoxy, heteroaralkoxy including 6-10 atoms, C₃-C₆ cycloalkoxy, C₃-C₆ cycloalkenyloxy, heterocyclyloxy including 3-6 atoms, or heterocycloalkenyloxy including 3-6 atoms, each of which is optionally substituted with from 1-5 R^{c}; C₁-C₆ thioalkoxy; C₁-C₆ thiohaloalkoxy; C₆-C₁₀ thioaryloxy or thioheteroaryloxy including 5-10 atoms, each of which is optionally substituted with from 1-10 R^{d}; C₇-C₁₀ thioaralkoxy, thioheteroaralkoxy including 6-10 atoms, C₃-C₆ thiocycloalkoxy, C₃-C₆ thiocycloalkenyloxy, thioheterocyclyloxy including 3-6 atoms, or thioheterocycloalkenyloxy including 3-6 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or

(iv) C₂-C₆ alkenyl or C₂-C₆ alkynyl, each of which is optionally substituted with from 1-10 R^{b}; or

(v) C₇-C₁₀ aralkyl, heteroaralkyl including 6-10 atoms, C₃-C₆ cycloalkyl, C₃-C₆ cycloalkenyl, heterocyclyl including 3-6 atoms, or heterocycloalkenyl including 3-6 atoms, each of which is optionally substituted with from 1-3 R^{c}; or

(vi) C₆-C₁₀ aryl or heteroaryl including 5-10 atoms, each of which is optionally substituted with from 1-10 R^{d};

R^{a} at each occurrence is, independently:

(i) NR^{m}Rⁿ; nitro; azido; hydroxy; oxo; thioxo; ═NR^{p}; C₁-C₂₀ alkoxy or C₁-C₂₀ haloalkoxy, each of which is optionally substituted with from 1-10 R^{a′}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or

(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{c};

R^{a′} at each occurrence is, independently, NR^{m}Rⁿ; nitro; azido; hydroxy; oxo; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; C₃-C₂₀ cycloalkyl, C₃-C₂₀ cycloalkenyl, heterocyclyl including 3-20 atoms, or heterocycloalkenyl including 3-20 atoms;

R^{b} at each occurrence is, independently:

(i) halo NR^{m}Rⁿ; nitro; azido; hydroxy; oxo; thioxo; ═NR^{p}; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or

(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{c}; or

(iii) C₆-C₁₈ aryl or heteroaryl including 5-16 atoms, each of which is optionally substituted with from 1-10 R^{d};

R^{c} at each occurrence is, independently:

(i) halo; NR^{m}Rⁿ; nitro; azido; hydroxy; oxo; thioxo; ═NR^{p}; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c′}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c′}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{b}; or

(iv) C₆-C₁₈ aryl or heteroaryl including 5-16 atoms, each of which is optionally substituted with from 1-10 R^{d}; or

(v) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{c′};

R^{c′} at each occurrence is, independently, R^{a′}; halo; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₁-C₂₀ alkyl or C₁-C₂₀ haloalkyl, each of which is optionally substituted with from 1-10 R^{a}; C₂-C₂₀ alkenyl; C₂-C₂₀ alkynyl; or C₆-C₁₈ aryl or heteroaryl including 5-16 atoms, each of which is optionally substituted with from 1-10 R^{d};

R^{d} at each occurrence is, independently:

(i) halo; NR^{m}Rⁿ; nitro; azido; hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d′}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d′}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{b}; or

(iv) C₇-C₂₀ aralkyl, heteroaralkyl including 6-20 atoms, C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{c}; or

(v) C₆-C₁₈ aryl or heteroaryl including 5-16 atoms, each of which is optionally substituted with from 1-10 R^{d′};

R^{d′} at each occurrence is, independently, halo; NR^{m}Rⁿ; nitro; azido; hydroxy; C₁-C₂₀ alkyl, C₁-C₂₀ haloalkyl, C₂-C₂₀ alkenyl; C₂-C₂₀ alkynyl; C₃-C₂₀ cycloalkyl; C₃-C₂₀ cycloalkenyl, heterocyclyl including 3-20 atoms; heterocycloalkenyl including 3-20 atoms; C₇-C₂₀ aralkyl; heteroaralkyl including 6-20 atoms; C₁-C₂₀ alkoxy; C₁-C₂₀ haloalkoxy; C₆-C₁₈ aryloxy; heteroaryloxy; C₇-C₂₀ aralkoxy; heteroaralkoxy including 6-20 atoms; C₃-C₁₆ cycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₇-C₂₀ thioaralkoxy, thioheteroaralkoxy including 6-20 atoms, C₃-C₁₆ thiocycloalkoxy C₃-C₂₀ thiocycloalkenyloxy, thioheterocyclyloxy including 3-20 atoms, or thioheterocycloalkenyloxy including 3-20 atoms; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ);

each of R^{e} at each occurrence is, independently, C₁-C₆ alkyl, optionally substituted with from 1-3 R^{a}; C₁-C₆haloalkyl; mercapto; C₁-C₆ thioalkoxy optionally substituted with from 1-3 R^{a}; C₆-C₁₀ aryl or C₆-C₁₀ aryloxy, each of which is optionally substituted with from 1-10 R^{d}; halo; hydroxyl; NR^{m}Rⁿ; nitro; C₂-C₆ alkenyl; C₂-C₆ alkynyl; C₁-C₆ alkoxy; C₁-C₆ haloalkoxy; cyano; —C(O)OR^{k}; or —C(O)R^{k};

R^{f} at each occurrence is, independently, mercapto; C₁-C₆ thioalkoxy optionally substituted with from 1-3 R^{e}; C₆-C₁₀ aryl or C₆-C₁₀ aryloxy, each of which is optionally substituted with from 1-10 R^{h}; halo; hydroxyl; NR^{m}Rⁿ; nitro; C₂-C₆ alkenyl; C₂-C₆ alkynyl; C₁-C₆ alkoxy; C₁-C₆ haloalkoxy; cyano; —C(O)OR^{k}; or —C(O)R^{k};

R^{g} at each occurrence is, independently:

(i) halo; NR^{m}Rⁿ; nitro; azido; hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ);

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{b};

R^{h} at each occurrence is, independently, hydroxyl; C₁-C₁₂ alkoxy; C₁-C₁₂ haloalkoxy; C₃-C₁₀ cycloalkoxy or C₃-C₁₀ cycloalkenyloxy, each of which is optionally substituted with from 1-5 R^{c}; or C₆-C₁₀ aryloxy or heteroaryloxy including 5-10 atoms, each of which is optionally substituted with from 1-5 R^{d};

each of R^{m}, Rⁿ, R^{o}, and R^{p}, at each occurrence is, independently:

(i) hydrogen; or

(ii) R¹⁰; or

(iii) heterocyclyl including 3-20 atoms or a heterocycloalkenyl including 3-20 atoms, each of which is optionally substituted with from 1-5 R^{c}; or

(iv) —C(O)R^{k}, —C(O)OR^{k}; or —S(O)ₙR^{q};

R^{k} at each occurrence is, independently:

(i) hydrogen; or

(ii) R¹⁰; or

(iii) heterocyclyl including 3-20 atoms or a heterocycloalkenyl including 3-20 atoms, each of which is optionally substituted with from 1-5 R^{c}; and

R^{q} at each occurrence is, independently, R^{k}, OR^{k}, or NR^{m}Rⁿ;

or an N-oxide and/or a salt (e.g., a pharmaceutically acceptable salt) thereof.

In another aspect, this invention features a compound having formula (VI):

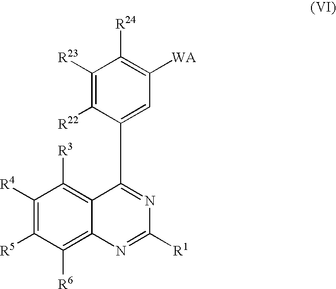

in which:

R¹ is:

(i) hydrogen; or

(ii) C₁-C₃ alkyl or C₁-C₃ haloalkyl; or

(iii) phenyl or heteroaryl including 5-6 atoms, each of which is optionally substituted with from 1-5 R^{d}; or

(iv) C₃-C₈ cycloalkyl or heterocyclyl including 3-8 atoms, each of which is optionally substituted with from 1-3 R^{c};

each of R³, R⁴, and R⁵ is, independently:

(i) hydrogen; or

(ii) halo; or

(iii) C₁-C₃ alkyl or C₁-C₃ haloalkyl, each of which is optionally substituted with from 1-3 R^{a}; or

(iv) C₃-C₆ cycloalkyl, which is optionally substituted with from 1-3 R^{c}; or

(v) C₆-C₁₀ aryl, which is optionally substituted with from 1-10 R^{d};

R⁶ is:

(i) halo; or

(ii) C₁-C₃ alkyl or C₁-C₃ haloalkyl, each of which is optionally substituted with from 1-3 R^{a} or R^{e}; or

(iii) cyano; —C(O)NR^{m}Rⁿ; or —S(O)ₙR^{q}, wherein n is 1 or 2;

(in certain embodiments, the definition of R⁶ can further include C₁-C₃ alkoxy);

each of R²², R²³, and R²⁴ is, independently, hydrogen or R^{e}; and

each of W and A can be as defined anywhere herein; or an N-oxide and/or a salt (e.g., a pharmaceutically acceptable salt) thereof.

Embodiments can include one or more of the following features.

R¹ can be hydrogen. R¹ can be C₁-C₃ alkyl.

R² can be phenyl, which is (a) substituted with 1 R⁷; and (b) optionally substituted with 1 R^{e}. In embodiments, R² can have formula (A-2):

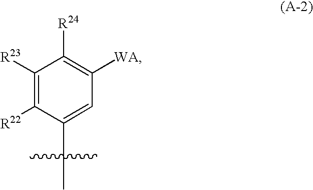

in which, one of R²², R²³, and R²⁴ (e.g., R²²) can be hydrogen or R^{e}, and the other two can be hydrogen.

In certain embodiments, each of R²², R²³, and R²⁴ can be hydrogen.

In other embodiments, R²² can be R^{e} (e.g., halo, e.g., chloro or fluoro) and each of R²³ and R²⁴ can be hydrogen.

W can be —O—, a bond, or —W¹(C₁₋₆ alkylene)- (e.g., W¹ can be O). For example, W can be —O—, a bond, or —OCH₂—.

A can be C₆-C₁₀ aryl, which is (a) substituted with 1 R⁹; and (b) optionally substituted with from 1-4 (e.g., 1) R^{g}. In embodiments, A can have formula (B-1):

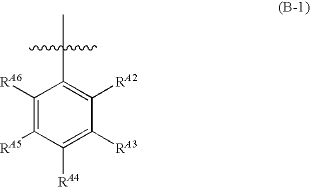

in which, one of R^{A3} and R^{A4} is R⁹, the other of R^{A3} and R^{A4} is hydrogen; and each of R^{A2}, R^{A5}, and R^{A6} can be, independently, hydrogen or R^{g}.

R⁹ can be —W²—S(O)ₙR¹⁰. In embodiments, R¹⁰ can be C₁-C₁₀ alkyl, optionally substituted with from 1-2 (e.g., 1) R^{a}. For example, R¹⁰ can be C₁-C₃ alkyl (e.g., methyl (CH₃), ethyl (CH₂CH₃), or isopropyl ((CH₃)₂CH), e.g., CH₃). As another example, R¹⁰ can be C₂-C₈ alkyl substituted with 1 R^{a}, in which R^{a} can be hydroxyl or C₁-C₃ alkoxy.

R² can have formula (C-1):

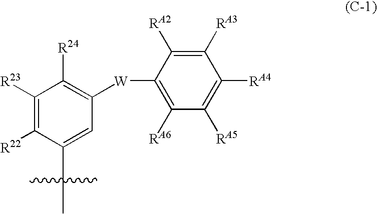

in which, one of R²², R²³, and R²⁴ is hydrogen or R^{e}, and the other two are hydrogen; one of R^{A3} and R^{A4} is R⁹, the other of R^{A3} and R^{A4} is hydrogen; and each of R^{A2}, R^{A5}, and R^{A6} is, independently, hydrogen or R^{g}.

In certain embodiments, each of R²², R²³, and R²⁴ can be hydrogen.

In other embodiments, R²² can be R^{e} (e.g., halo, e.g., chloro or fluoro), and each of R²³ and R²⁴ can be hydrogen.

W can be —O—, a bond, or —OCH₂—.

R^{A3} can be —W²—S(O)ₙR¹⁰, in which W² is a bond and n is 2.

R¹⁰ can be C₁-C₁₀ alkyl, optionally substituted with from 1-2 (e.g., 1) R^{a}. In certain embodiments, R¹⁰ can be C₁-C₃ alkyl (e.g., methyl (CH₃), ethyl (CH₂CH₃), or isopropyl[(CH₃)₂CH], e.g., R¹⁰ can be CH₃). In other embodiments, R¹⁰ can be C₂-C₈ alkyl substituted with 1 R^{a}, in which R^{a} can be hydroxyl or C₁-C₃ alkoxy.

R^{A5} can be hydrogen or R^{g}, and each of R^{A2} and R^{A6} is hydrogen.

Each of R^{A2}, R^{A5}, and R^{A6} can be hydrogen.

R³, R⁴, and R⁵ can each be hydrogen.

R⁶ can be C₁-C₆ haloalkyl (e.g., C₁-C₃ perhaloalkyl, e.g., CF₃).

R⁶ can be halo (e.g., chloro). R⁶ can be cyano or C₁-C₃ alkoxy (e.g., OCH₃).

R¹ can be hydrogen. R¹ can be C₁-C₃ alkyl or C₁-C₃ haloalkyl. For example, R¹ can be CH₃ or CF₃.

R¹ can be C₆-C₁₀ aryl or heteroaryl including 5-10 atoms, each of which is optionally substituted with from 1-5 R^{d}. In certain embodiments, R¹ can be phenyl, which is optionally substituted with from 1-5 R^{d}. In other embodiments, R¹ can be thienyl, which is optionally substituted with from 1-5 R^{d}.

R¹ can be C₃-C₈ cycloalkyl or heterocyclyl including 3-8 atoms, each of which is optionally substituted with from 1-3 R^{c}.

R² can be C₆-C₁₀ aryl, which is (a) substituted with from 1-2 R⁷; and (b) optionally substituted with from 1-2 R^{e}. For example, R² can be phenyl, which is (a) substituted with 1 R⁷; and (b) optionally substituted with 1 R^{e}. In certain embodiments, R² can be phenyl, which is substituted with 1 R⁷.

R² can have formula (A-2):

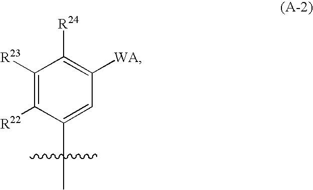

each of R²², R²³, and R²⁴ can be, independently, hydrogen or R^{e}.

In certain embodiments, each of R²², R²³, and R²⁴ can be hydrogen. In certain embodiments, one of R²², R²³, and R²⁴ can be R^{e}, and the other two can be hydrogen.

In other embodiments, R²² can be R^{e}, and each of R²³ and R²⁴ can be hydrogen.

In some embodiments, R²² can be halo. For example, R²² can be chloro.

W can be —O—, a bond, or —W¹(C₁₋₆ alkylene)- (e.g., W¹ can be O). For example, W can be —O—, a bond, or —OCH₂—.

In some embodiments, A can be C₆-C₁₀ aryl, which is (a) substituted with from 1-2 R⁹; and (b) optionally substituted with from 1-4 R^{g}. In other embodiments, A can be C₆-C₁₀ aryl, which is (a) substituted with 1 R⁹; and (b) optionally substituted with from 1-4 R^{g}. For example, A can be phenyl, which is (a) substituted with 1 R⁹; and (b) optionally substituted with from 1-4 R^{g}.

In some embodiments, A can have formula (B-1):

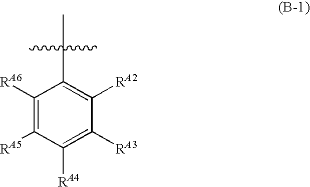

in which:

one of R^{A3} and R^{A4} can be R⁹, the other of R^{A3} and R^{A4} can be hydrogen; and

each of R^{A2}, R^{A5}, and R^{A6} can be, independently, hydrogen or R^{g}. In certain embodiments, R⁹ can be —W²—S(O)ₙR¹⁰. In other embodiments, R⁹ can be —W²—C(O)OR¹³.

In some embodiments, R² can have formula (C-1):

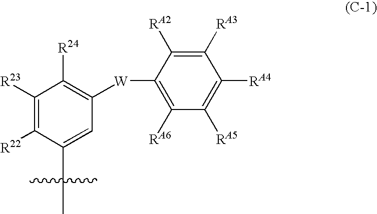

in which each of R²², R²³, and R²⁴ can be, independently, hydrogen or R^{e}; and one of R^{A2}, R^{A3}, R^{A4}, R^{A5}, and R^{A6} can be R⁹, and the others can each be, independently, hydrogen or R^{g}.

In certain embodiments, each of R²², R²³, and R²⁴ can be hydrogen. In other embodiments one of R²², R²³, and R²⁴ can be R^{e}, and the other two can be hydrogen. In some embodiments, R²² can be R^{e}, and each of R²³ and R²⁴ can be hydrogen. In certain embodiments, R²² can be halo. For example, R²² can be chloro.

W can be —O—, a bond, or —OCH₂—.

One of R^{A3} and R^{A4} can be R⁹, and the other of R^{A3} and R^{A4} can be hydrogen; and each of R^{A2}, R^{A5}, and R^{A6} can be, independently, hydrogen or R^{g}.

R^{A3} can be —W²—S(O)ₙR¹⁰. In certain embodiments, each of R^{A2}, R^{A5}, and R^{A6} can be hydrogen, and W² can be a bond. In some embodiments, n can be 2.

R¹⁰ can be C₁-C₁₀ alkyl, optionally substituted with from 1-2 R^{a}. For example, R¹⁰ can be C₁-C₃ alkyl, e.g., CH₃. In certain embodiments, R¹⁰ can be C₂-C₈ alkyl substituted with 1 R^{a}. In embodiments, R^{a} can be hydroxyl or C₁-C₃ alkoxy.

R^{A5} can be hydrogen or R^{g}, and each of R^{A2} and R^{A6} can be hydrogen.

R^{A4} can be —W²—C(O)OR¹³. In embodiments, R¹³ can be hydrogen or C₁-C₃ alkyl. In embodiments, W² can be C₁-C₃ alkylene. For example, W² can be CH₂. In other embodiments, W² can be a bond. In some embodiments, each of R^{A2}, R^{A5}, and R^{A6} can be hydrogen.

Each of R³, R⁴, and R⁵ can be, independently:

(i) hydrogen; or

(ii) halo; or

(iii) C₁-C₆ alkyl or C₁-C₆ haloalkyl, each of which is optionally substituted with from 1-3 R^{e}; or

(iv) C₃-C₆ cycloalkyl, which is optionally substituted with from 1-3 R^{e}; or

(v) C₆-C₁₀ aryl, which is optionally substituted with from 1-10 R^{d}.

In certain embodiments, R³, R⁴, and R⁵ can be hydrogen.

In some embodiments, R⁶ can be:

(i) halo; or

(ii) C₁-C₆ alkyl or C₁-C₆ haloalkyl, each of which is optionally substituted with from 1-3 R^{e}; or

(iii) cyano; —C(O)NR^{m}Rⁿ; or —S(O)ₙR^{q}, wherein n is 1 or 2.

(in certain embodiments, the definition of R⁶ can further include C₁-C₃ alkoxy);

In some embodiments, R⁶ can be C₁-C₆ haloalkyl. For example, R⁶ can be C₁-C₃ perfluoroalkyl, e.g., CF₃.

In some embodiments, R⁶ can be halo, e.g., chloro.

In some embodiments, the compound can have formula (VI):

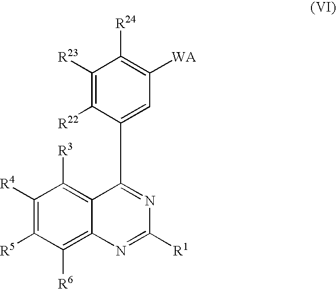

in which:

R¹ can be:

(i) hydrogen; or

(ii) C₁-C₃ alkyl or C₁-C₃ haloalkyl; or

(iii) phenyl or heteroaryl including 5-6 atoms, each of which is optionally substituted with from 1-5 R^{d}; or

(iv) C₃-C₈ cycloalkyl or heterocyclyl including 3-8 atoms, each of which is optionally substituted with from 1-3 R^{c};

each of R³, R⁴, and R⁵ can be, independently:

(i) hydrogen; or

(ii) halo; or

(iii) C₁-C₃ alkyl or C₁-C₃ haloalkyl, each of which is optionally substituted with from 1-3 R^{a}; or

(iv) C₃-C₆ cycloalkyl, which is optionally substituted with from 1-3 R^{c}; or

(v) C₆-C₁₀ aryl, which is optionally substituted with from 1-10 R^{d};

R⁶ can be:

(i) halo; or

(ii) C₁-C₃ alkyl or C₁-C₃ haloalkyl, each of which is optionally substituted with from 1-3 R^{a}; or

(iii) cyano; —C(O)NR^{m}Rⁿ; or —S(O)ₙR^{q}, wherein n is 1 or 2; and

(in certain embodiments, the definition of R⁶ can further include C₁-C₃ alkoxy);

each of R²², R²³, and R²⁴ can be, independently, hydrogen or R^{e}.

R¹ can be hydrogen. R¹ can be CH₃ or CF₃. R¹ can be phenyl or thienyl, each of which is optionally substituted with from 1-5 R^{d}.

W can be —O—, a bond, or —OCH₂—.

A can have formula (B-1), in which one of R^{A3} and R^{A4} can be R⁹, and the other of R^{A3} and R^{A4} can be hydrogen; and each of R^{A2}, R^{A5}, and R^{A6} can be, independently, hydrogen or R^{g}.

In some embodiments, R^{A3} can be —W²—S(O)ₙR¹⁰, in which W² can be a bond, and n can be 2. In embodiments, R¹⁰ can be C₁-C₁₀ alkyl, optionally substituted with from 1-2 R^{a}. For example, R¹⁰ can be CH₃, CH₂CH₃, or isopropyl. As another example, R¹⁰ can be C₂-C₈ alkyl substituted with 1 R^{a}. In embodiments, R^{a} can be hydroxyl or C₁-C₃ alkoxy. In certain embodiments, R^{A5} can be hydrogen or R^{g}, and each of R^{A2} and R^{A6} can be hydrogen.

In some embodiments, R^{A4} can be —W²—C(O)OR¹³. In embodiments, R¹³ can be hydrogen or C₁-C₃ alkyl. In certain embodiments, W² can be CH₂. In some embodiments, each of R^{A2}, R^{A5}, and R^{A6} can be hydrogen.

Each of R³, R⁴, and R⁵ can be hydrogen.

Each of R²², R²³, and R²⁴ can be hydrogen.

One of R²², R²³, and R²⁴ can be R^{e}, and the other two can be hydrogen. In certain embodiments, R²² can be R^{e}, and each of R²³ and R²⁴ can be hydrogen. In certain embodiments, R²² can be chloro.

In some embodiments, R⁶ can be CF₃. In other embodiments, R⁶ can be chloro.

In some embodiments, R^{a} at each occurrence can be, independently:

(i) NR^{m}Rⁿ; hydroxy; C₁-C₆ alkoxy or C₁-C₆ haloalkoxy; C₆-C₁₀ aryloxy or heteroaryloxy including 5-10 atoms, each of which is optionally substituted with from 1-5 R^{d}; C₇-C₁₁ aralkoxy, heteroaralkoxy including 6-11 atoms, C₃-C₁₁ cycloalkoxy, C₃-C₁₁ cycloalkenyloxy, heterocyclyloxy including 3-10 atoms, or heterocycloalkenyloxy including 3-10 atoms, each of which is optionally substituted with from 1-5 R^{c}; or cyano; or

(ii) C₃-C₁₀ cycloalkyl, C₃-C₁₀ cycloalkenyl, heterocyclyl including 3-10 atoms, or heterocycloalkenyl including 3-10 atoms, each of which is optionally substituted with from 1-5 R^{c}.

In some embodiments, R^{a} at each occurrence can be, independently:
NR^{m}Rⁿ; hydroxy; C₁-C₆ alkoxy or C₁-C₆ haloalkoxy;
C₆-C₁₀ aryloxy or heteroaryloxy including 5-10 atoms, each of which is optionally substituted with from 1-5 substituents independently selected from halo; NR^{m}Rⁿ; hydroxy; C₁-C₆ alkoxy; C₁-C₆ haloalkoxy; cyano; C₁-C₆ alkyl; C₁-C₆ haloalkyl; C₂-C₆ alkenyl or C₂-C₆ alkynyl; or
C₇-C₁₁ aralkoxy, heteroaralkoxy including 6-11 atoms, C₃-C₁₁ cycloalkoxy, C₃-C₁₁ cycloalkenyloxy, heterocyclyloxy including 3-10 atoms, or heterocycloalkenyloxy including 3-10 atoms, each of which is optionally substituted with from 1-5 substituents independently selected from halo; NR^{m}Rⁿ; hydroxy; C₁-C₆ alkoxy; C₁-C₆ haloalkoxy; C₁-C₆ alkyl; C₁-C₆ haloalkyl; C₂-C₆ alkenyl or C₂-C₆ alkynyl; or
C₃-C₁₀ cycloalkyl, C₃-C₁₀ cycloalkenyl, heterocyclyl including 3-10 atoms, or heterocycloalkenyl including 3-10 atoms, each of which is optionally substituted with from 1-5 substituents independently selected from halo; NR^{m}Rⁿ; hydroxy; C₁-C₆ alkoxy; C₁-C₆ haloalkoxy; C₁-C₆ alkyl; C₁-C₆ haloalkyl; C₂-C₆ alkenyl or C₂-C₆ alkynyl.

In some embodiments, R^{b} at each occurrence can be, independently:

(i) halo; NR^{m}Rⁿ; hydroxy; C₁-C₆ alkoxy or C₁-C₆ haloalkoxy; C₆-C₁₀ aryloxy or heteroaryloxy including 5-10 atoms, each of which is optionally substituted with from 1-5 R^{d}; C₇-C₁ aralkoxy, heteroaralkoxy including 6-11 atoms, C₃-C₁₀ cycloalkoxy, C₃-C₁₀ cycloalkenyloxy, heterocyclyloxy including 3-10 atoms, or heterocycloalkenyloxy including 3-10 atoms, each of which is optionally substituted with from 1-5 R^{c};

(ii) C₃-C₁₀ cycloalkyl, C₃-C₁₀ cycloalkenyl, heterocyclyl including 3-10 atoms, or heterocycloalkenyl including 3-10 atoms, each of which is optionally substituted with from 1-5 R^{c}; or

(iii) C₆-C₁₀ aryl or heteroaryl including 5-10 atoms, each of which is optionally substituted with from 1-5 R^{d}.

In some embodiments, R^{b} at each occurrence can be, independently:
halo; NR^{m}Rⁿ; hydroxy; C₁-C₆ alkoxy or C₁-C₆ haloalkoxy;
C₆-C₁₀ aryloxy or heteroaryloxy including 5-10 atoms, each of which is optionally substituted with from 1-5 substituents independently selected from halo; NR^{m}Rⁿ; hydroxy; C₁-C₆ alkoxy; C₁-C₆ haloalkoxy; cyano; C₁-C₆ alkyl; C₁-C₆ haloalkyl; C₂-C₆ alkenyl or C₂-C₆ alkynyl; or
C₇-C₁₁ aralkoxy, heteroaralkoxy including 6-11 atoms, C₃-C₁₀ cycloalkoxy, C₃-C₁₀ cycloalkenyloxy, heterocyclyloxy including 3-10 atoms, or heterocycloalkenyloxy including 3-10 atoms, each of which is optionally substituted with from halo; NR^{m}Rⁿ; hydroxy; C₁-C₆ alkoxy; C₁-C₆ haloalkoxy; C₁-C₆ alkyl; C₁-C₆ haloalkyl; C₂-C₆ alkenyl or C₂-C₆ alkynyl; or
C₃-C₁₀ cycloalkyl, C₃-C₁₀ cycloalkenyl, heterocyclyl including 3-10 atoms, or heterocycloalkenyl including 3-10 atoms, each of which is optionally substituted with from halo; NR^{m}Rⁿ; hydroxy; C₁-C₆ alkoxy; C₁-C₆ haloalkoxy; C₁-C₆ alkyl; C₁-C₆ haloalkyl; C₂-C₆ alkenyl or C₂-C₆ alkynyl; or
C₆-C₁₀ aryl or heteroaryl including 5-10 atoms, each of which is optionally substituted with from 1-5 substituents independently selected from halo; NR^{m}Rⁿ; hydroxy; C₁-C₆ alkoxy; C₁-C₆ haloalkoxy; cyano; C₁-C₆ alkyl; C₁-C₆ haloalkyl; C₂-C₆ alkenyl or C₂-C₆ alkynyl.

In some embodiments, R^{c} at each occurrence can be, independently:

(i) halo; NR^{m}Rⁿ; hydroxy; C₁-C₆ alkoxy or C₁-C₆ haloalkoxy; or

(ii) C₁-C₆ alkyl or C₁-C₆ haloalkyl, each of which is optionally substituted with from 1-5 R^{a} (R^{a} can be as defined anywhere herein); or

(iii) C₂-C₆ alkenyl or C₂-C₆ alkynyl, each of which is optionally substituted with from 1-5 R^{b} (R^{b} can be as defined anywhere herein).

In some embodiments, R^{d} at each occurrence can be, independently:

(i) halo; NR^{m}Rⁿ; hydroxy; C₁-C₆ alkoxy or C₁-C₆ haloalkoxy; or cyano; or

(ii) C₁-C₆ alkyl or C₁-C₆ haloalkyl, each of which is optionally substituted with from 1-5 R^{a} (R^{a} can be as defined anywhere herein); or

(iii) C₂-C₆ alkenyl or C₂-C₆ alkynyl, each of which is optionally substituted with from 1-5 R^{b} (R^{b} can be as defined anywhere herein).

In some embodiments, R^{e} at each occurrence is, independently, C₁-C₆ alkyl; C₁-C₆ haloalkyl; halo; hydroxyl; NR^{m}Rⁿ; C₁-C₆ haloalkoxy; or cyano.

In some embodiments, R^{g} at each occurrence is, independently:

(i) halo; NR^{m}Rⁿ; hydroxy; C₁-C₆ alkoxy or C₁-C₆ haloalkoxy; cyano; or

(ii) C₁-C₆ alkyl or C₁-C₆ haloalkyl.

In some embodiments, R^{h} at each occurrence is, independently, hydroxyl, C₁-C₆ alkoxy, or C₁-C₆ haloalkoxy; C₃-C₁₀ cycloalkoxy or C₃-C₁₀ cycloalkenyloxy, each of which is optionally substituted with from 1-5 R^{c}; or C₆-C₁₀ aryloxy or heteroaryloxy including 5-10 atoms, each of which is optionally substituted with from 1-5 R^{d}.

In some embodiments, each of R^{m} and Rⁿ at each occurrence is, independently, hydrogen, C₁-C₆ alkyl, or C₁-C₆ haloalkyl.

In one aspect, this invention relates to any of the specific quinazoline compounds delineated herein (e.g., as shown in the Examples).

In one aspect, this invention features a pharmaceutical composition, which includes a compound of formula (I) (including any subgenera or specific compounds thereof) or a salt (e.g., a pharmaceutically acceptable salt) or a prodrug thereof and a pharmaceutically acceptable adjuvant, carrier or diluent. In some embodiments, the composition can include an effective amount of the compound or the salt thereof. In some embodiments, the composition can further include an additional therapeutic agent.

The invention also relates generally to modulating (e.g., activating) LXRs with the quinazoline compounds described herein. In some embodiments, the methods can include, e.g., contacting an LXR in a sample (e.g., a tissue, a cell free assay medium, a cell-based assay medium) with a compound of formula (I) (including any subgenera or specific compounds thereof). In other embodiments, the methods can include administering a compound of formula (I) (including any subgenera or specific compounds thereof) to a subject (e.g., a mammal, e.g., a human, e.g., a human having or at risk of having one or more of the diseases or disorders described herein).

In one aspect, this invention features a method of selectively modulating (e.g., activating) LXRβ (e.g., selectively modulating LXRβ relative to LXRα, e.g., selectively activating LXRβ relative to LXRα). In some embodiments, a compound of formula (I) can have an LXRα/LXRβ binding ratio of from about 1.5 to about 1,000 (e.g., from about 1.5 to about 500, from about 1.5 to about 100, from about 5.0 to about 100, from about 5.0 to about 70, from about 5 to about 60, from about 5 to about 50, from about 5 to about 20, from about 10 to about 70, from about 20 to about 70, from about 30 to about 70, from about 40 to about 70, from about 50 to about 70, from about 60 to about 70, or from about 30 to about 70).

As used herein, the term “LXRα/LXRβ binding ratio” refers to the following ratio: IC50 (μM) LXRα binding/IC50 (μM) LXRβ binding.

In certain embodiments, a compound of formula (I) can have an LXRα/LXRβ binding ratio of from about 5 to about 20; from about 30 to about 39; from about 40 to about 45; or from about 54 to about 60.

In one aspect, this invention also relates generally to methods of treating (e.g., controlling, ameliorating, preventing, delaying the onset of, or reducing the risk of developing) one or more LXR-mediated diseases or disorders in a subject (e.g., a subject in need thereof). The methods include administering to the subject an effective amount of a compound of formula (I) (including any subgenera or specific compounds thereof) or a pharmaceutically acceptable salt or prodrug thereof. LXR-mediated diseases or disorders can include, e.g., cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging or connective tissue diseases.

In another aspect, this invention relates to methods of modulating (e.g., increasing) serum HDL cholesterol levels in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., decreasing) serum LDL cholesterol levels in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., decreasing) serum triglyceride levels in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., increasing) reverse cholesterol transport in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., decreasing or inhibiting) cholesterol absorption in a subject (e.g., a subject in need thereof), which includes administering to the subject an effective amount of a compound of formula (I) (including any subgenera or specific compounds thereof) or a pharmaceutically acceptable salt or prodrug thereof.

In a further aspect, this invention relates to methods of preventing or treating a cardiovascular disease (e.g., acute coronary syndrome, restenosis), which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating a atherosclerosis and/or atherosclerotic lesions, which includes administering to a subject in need thereof an effective amount of a compound of formula (I) (including any subgenera or specific compounds thereof) or a pharmaceutically acceptable salt or prodrug thereof.

In another aspect, this invention relates to methods of preventing or treating diabetes (e.g., type I diabetes or type 2 diabetes), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating Syndrome X, which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating a obesity, which includes administering to a subject in need thereof an effective amount of a compound of formula (I) (including any subgenera or specific compounds thereof) or a pharmaceutically acceptable salt or prodrug thereof.

In another aspect, this invention relates to methods of preventing or treating a lipid disorder (e.g., dyslipidemia, hyperlipidemia, hypertriglyceridemia, hypercholesterolemia, low HDL and high LDL), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating a cognitive disorder (e.g., Alzheimer's disease or dementia), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating a Alzheimer's disease or dementia, which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating a Alzheimer's disease, which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating an inflammatory disease (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating celiac, which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating thyroiditis, which includes administering to a subject in need thereof an effective amount of a compound of formula (I) (including any subgenera or specific compounds thereof) or a pharmaceutically acceptable salt or prodrug thereof.

In one aspect, this invention relates to methods of treating a connective tissue disease (e.g., osteoarthritis or tendonitis), which includes administering to a subject (e.g., a mammal, e.g., a human) in need thereof an effective amount of a compound of formula (I) (including any subgenera or specific compounds thereof) or a pharmaceutically acceptable salt or prodrug thereof. In embodiments, the compound of formula (I) inhibits (e.g., reduces or otherwise diminishes) cartilage degradation. In embodiments, the compound of formula (I) induces (e.g., increases or otherwise agments) cartilage regeneration. In embodiments, the compound of formula (I) inhibits (e.g., reduces or otherwise diminishes) cartilage degradation and induces (e.g., increases or otherwise agments) cartilage regeneration. In embodiments, the compound of formula (I) inhibits (e.g., reduces or otherwise diminishes) aggrecanase activity. In embodiments, the compound of formula (I) inhibits (e.g., reduces or otherwise diminishes) elaboration of pro-inflammatory cytokines in osteoarthritic lesions.

In another aspect, this invention relates to methods of treating or preventing skin aging, the method comprising administering (e.g., topically administering) to a subject (e.g., a mammal, e.g., a human) in need thereof an effective amount of a compound of formula (I) (including any subgenera or specific compounds thereof) or a pharmaceutically acceptable salt or prodrug thereof. In embodiments, the skin aging can be derived from chronological aging, photoaging, steroid-induced skin thinning, or a combination thereof.

The term “skin aging” includes conditions derived from intrinsic chronological aging (for example, deepened expression lines, reduction of skin thickness, inelasticity, and/or unblemished smooth surface), those derived from photoaging (for example, deep wrinkles, yellow and leathery surface, hardening of the skin, elastosis, roughness, dyspigmentations (age spots) and/or blotchy skin), and those derived from steroid-induced skin thinning. Accordingly, another aspect is a method of counteracting UV photodamage, which includes contacting a skin cell exposed to UV light with an effective amount of a compound of formula (I).

In some embodiments, the compound of formula (I) (including any subgenera or specific compounds thereof) does not substantially increase serum and/or hepatic triglyceride levels of the subject.

In some embodiments, the administered compound of formula (I) (including any subgenera or specific compounds thereof) can be an LXR agonist (e.g., an LXRα agonist or an LXRβ agonist, e.g., an LXRβ agonist).

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In a further aspect, this invention also relates to methods of making compounds described herein. Alternatively, the method includes taking any one of the intermediate compounds described herein and reacting it with one or more chemical reagents in one or more steps to produce a compound described herein.

In one aspect, this invention relates to a packaged product. The packaged product includes a container, one of the aforementioned compounds in the container, and a legend (e.g., a label or an insert) associated with the container and indicating administration of the compound for treatment and control of the diseases or disorders described herein.

The term “mammal” includes organisms, which include mice, rats, cows, sheep, pigs, rabbits, goats, horses, monkeys, dogs, cats, and humans.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/Kg to about 1000 mg/Kg, (e.g., from about 0.1 mg/Kg to about 100 mg/Kg, from about 1 mg/Kg to about 100 mg/Kg). Effective doses will also vary depending on route of administration, as well as the possibility of co-usage with other agents.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, polycyclic rings.

The term “alkyl” refers to a saturated hydrocarbon chain that may be a straight chain or branched chain, containing the indicated number of carbon atoms. For example, C₁-C₂₀ alkyl indicates that the group may have from 1 to 20 (inclusive) carbon atoms in it. Any atom can be optionally substituted, e.g., by one or more substitutents. Examples of alkyl groups include without limitation methyl, ethyl, n-propyl, isopropyl, and tert-butyl.

The term “cycloalkyl” refers to saturated monocyclic, bicyclic, tricyclic, or other polycyclic hydrocarbon groups. Any atom can be optionally substituted, e.g., by one or more substituents. A ring carbon serves as the point of attachment of a cycloalkyl group to another moiety. Cycloalkyl groups can contain fused rings. Cycloalkyl moieties can include, e.g., cyclopropyl, cyclobutyl, cyclopentyl, cyclohexyl, cycloheptyl, adamantyl, and norbornyl(bicycle[2.2.1]heptyl).

The terms “alkylene,” “alkenylene,” “alkynylene,” and “cycloalkylene” refer to divalent straight chain or branched chain alkyl (e.g., —CH₂—), alkenyl (e.g., —CH═CH—), alkynyl (e.g., —C [symbol] —); or cycloalkyl moieties, respectively.

The term “haloalkyl” refers to an alkyl group, in which at least one hydrogen atom is replaced by halo. In some embodiments, more than one hydrogen atom (2, 3, 4, 5, 6, 7, 8, 9, 10, 11, 12, 13, 14, 15, 16, 17, 18, 19, 20, 21, 22, 23, 24, 25, 26, etc. hydrogen atoms) on a alkyl group can be replaced by more than one halogen (e.g., 2, 3, 4, 5, 6, 7, 8, 9, 10, 11, 12, 13, 14, 15, 16, 17, 18, 19, 20, 21, 22, 23, 24, 25, 26, etc. halogen atoms). In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (e.g., perhaloalkyl, e.g., perfluoroalkyl, such as trifluoromethyl). Any atom can be optionally substituted, e.g., by one or more substituents.

The term “aralkyl” refers to an alkyl moiety in which an alkyl hydrogen atom is replaced by an aryl group. One of the carbons of the alkyl moiety serves as the point of attachment of the aralkyl group to another moiety. Aralkyl includes groups in which more than one hydrogen atom on an alkyl moiety has been replaced by an aryl group. Any ring or chain atom can be optionally substituted, e.g., by one or more substituents. Non-limiting examples of “aralkyl” include benzyl, 2-phenylethyl, 3-phenylpropyl, benzhydryl(diphenylmethyl), and trityl(triphenylmethyl) groups.

The term “heteroaralkyl” refers to an alkyl moiety in which an alkyl hydrogen atom is replaced by a heteroaryl group. One of the carbons of the alkyl moiety serves as the point of attachment of the aralkyl group to another moiety. Heteroaralkyl includes groups in which more than one hydrogen atom on an alkyl moiety has been replaced by a heteroaryl group. Any ring or chain atom can be optionally substituted e.g., by one or more substituents. Heteroaralkyl can include, for example, 2-pyridylethyl.

The term “alkenyl” refers to a straight or branched hydrocarbon chain containing 2-20 carbon atoms and having one or more double bonds. Any atom can be optionally substituted, e.g., by one or more substituents. Alkenyl groups can include, e.g., allyl, 1-butenyl, 2-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be optionally substituted, e.g., by one or more substituents. Alkynyl groups can include, e.g., ethynyl, propargyl, and 3-hexynyl. One of the triple bond carbons can optionally be the point of attachment of the alkynyl substituent.

The term “alkoxy” refers to an —O-alkyl radical. The term “mercapto” refers to an SH radical. The term “thioalkoxy” refers to an —S-alkyl radical. The terms “aryloxy” and “heteroaryloxy” refer to an —O-aryl radical and —O-heteroaryl radical, respectively. The terms “thioaryloxy” and “thioheteroaryloxy” refer to an —S-aryl radical and —S-heteroaryl radical, respectively.

The terms “aralkoxy” and “heteroaralkoxy” refer to an —O-aralkyl radical and —O-heteroaralkyl radical, respectively. The terms “thioaralkoxy” and “thioheteroaralkoxy” refer to an —S-aralkyl radical and —S-heteroaralkyl radical, respectively. The term “cycloalkoxy” refers to an —O-cycloalkyl radical. The terms “cycloalkenyloxy” and “heterocycloalkenyloxy” refer to an —O-cycloalkenyl radical and —O-heterocycloalkenyl radical, respectively. The term “heterocyclyloxy” refers to an —O-heterocyclyl radical. The term “thiocycloalkoxy” refers to an —S-cycloalkyl radical. The terms “thiocycloalkenyloxy” and “thioheterocycloalkenyloxy” refer to an —S-cycloalkenyl radical and —S-heterocycloalkenyl radical, respectively. The term “thioheterocyclyloxy” refers to an —S-heterocyclyl radical.

The term “heterocyclyl” refers to a saturated monocyclic, bicyclic, tricyclic or other polycyclic ring system having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂). Thus, a heterocyclyl ring includes carbon atoms and 1-4, 1-8, or 1-10 heteroatoms selected from N, O, or S if monocyclic, bicyclic, or tricyclic, respectively. A ring heteroatom or ring carbon is the point of attachment of the heterocyclyl substituent to another moiety. Any atom can be optionally substituted, e.g., by one or more substituents. The heterocyclyl groups can contain fused rings. Heterocyclyl groups can include, e.g., tetrahydrofuryl, tetrahydropyranyl, piperidyl(piperidino), piperazinyl, morpholinyl(morpholino), pyrrolinyl, and pyrrolidinyl.

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted e.g., by one or more substituents. The cycloalkenyl groups can contain fused rings. Fused rings are rings that share a common carbon atom. Cycloalkenyl moieties can include, e.g., cyclohexenyl, cyclohexadienyl, or norbornenyl.

The term “heterocycloalkenyl” refers to partially unsaturated monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). A ring carbon (e.g., saturated or unsaturated) or heteroatom is the point of attachment of the heterocycloalkenyl substituent. Any atom can be optionally substituted, e.g., by one or more substituents. The heterocycloalkenyl groups can contain fused rings. Heterocycloalkenyl groups can include, e.g., tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H[1,3]oxazinyl.

The term “aryl” refers to a fully unsaturated, aromatic monocyclic, bicyclic, or tricyclic, hydrocarbon ring system, wherein any ring atom can be optionally substituted, e.g., by one or more substituents. Aryl groups can contain fused rings. Aryl moieties can include, e.g., phenyl, naphthyl, anthracenyl, and pyrenyl.

The term “heteroaryl” refers to a fully unsaturated, aromatic monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms independently selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). Any atom can be optionally substituted, e.g., by one or more substituents. Heteroaryl groups can contain fused rings. Heteroaryl groups can include, e.g., pyridyl, thienyl, furyl(furanyl), imidazolyl, indolyl, isoquinolyl, quinolyl and pyrrolyl.

Descriptors such as C(O), C(S), and C(NRⁱ) refer to carbon atoms that are doubly bonded to an oxygen, sulfur, and nitrogen atom, respectively.

The term “substituent” refers to a group “substituted” on, e.g., an alkyl, haloalkyl, cycloalkyl, alkenyl, alkynyl, aralkyl, heteroaralkyl, heterocyclyl, heterocycloalkenyl, cycloalkenyl, aryl, or heteroaryl group at any atom of that group. In one aspect, the substituent(s) (e.g., R^{a}) on a group are independently any one single, or any combination of two or more of the permissible atoms or groups of atoms delineated for that substituent. In another aspect, a substituent may itself be substituted with any one of the above substituents.

In general, when a definition for a particular variable includes both hydrogen and non-hydrogen (halo, alkyl, aryl, etc.) possibilities, the term “substituent(s) other than hydrogen” refers collectively to the non-hydrogen possibilities for that particular variable.

Descriptors such as “alkyl” which is optionally substituted with from 1-10 R^{a}” (and the like) is intended to mean both an unsubstituted alkyl group and an alkyl group that is substituted with from 1-10 R^{a}. The use of (radical) prefix names, such as alkyl without the modifier “optionally substituted” or “substituted” is understood to mean that the particular group is unsubstituted. However, the use of “haloalkyl” without the modifier “optionally substituted” or “substituted” is still understood to mean an alkyl group, in which at least one hydrogen atom is replaced by halo.

In some embodiments, the compounds have agonist activity for genes involved with HDL production and cholesterol efflux (e.g., ABCA1) and antagonist activity for genes involved with triglyceride synthesis (e.g., SREBP-1c).

The details of one or more embodiments of the invention are set forth in the description below. Other features and advantages of the invention will be apparent from the description and from the claim.

### DETAILED DESCRIPTION

This invention relates generally to quinazoline-based modulators of Liver X receptors (LXRs) and related methods.

The quinazoline-based LXR modulators have the general formula (I):

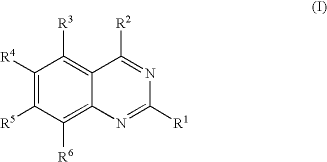

in which R¹, R², R³, R⁴, R⁵, R⁶, R⁷, R⁸, R⁹, R¹⁰, R¹¹, R¹², R¹³, R¹⁴, R¹⁵, W, W¹, W², W³, A, R^{a}, R^{a′}, R^{b}, R^{b′}, R^{c}, R^{d}, R^{d′}, R^{e}, R^{f}, R^{g}, R^{h}, Rⁱ, R^{j}, R^{k}, R^{m}, Rⁿ, R^{o}, R^{p}, R^{q}, and n, can be, independently, as defined anywhere herein.

For ease of exposition, it is understood that where in this specification (including the claims), a group is defined by “as defined anywhere herein” (or the like), the definitions for that particular group include the first occurring and broadest generic definition as well as any sub-generic and specific definitions delineated anywhere in this specification.

Also, for ease of exposition, it is understood that any recitation of ranges (e.g., C₁-C₁₂, 1-4) or sub-ranges of a particular range (e.g., C₁-C₄, C₂-C₆, 1-2) for any of R¹, R², R³, R⁴, R⁵, R⁶, R⁷, R⁸, R⁹, R¹⁰, R¹¹, R¹², R¹³, R¹⁴, R¹⁵, W, W¹, W², A, R^{a}, R^{a′}, R^{b}, R^{b′}, R^{c}, R^{d}, R^{d′}, R^{e}, R^{f}, R^{g}, R^{h}, Rⁱ, R^{j}, R^{k}, R^{m}, Rⁿ, R^{o}, R^{p}, R^{q}, and n expressly includes each of the individual values that fall within the recited range, including the upper and lower limits of the recited range. For example, the range C₁-C₄ alkyl is understood to mean C₁, C₂, C₃, C₄, C₁-C₄, C₁-C₃, C₁-C₂, C₂-C₄, C₂-C₃, or C₃-C₄ alkyl and the range 1-3 R^{a} is understood to mean 1, 2, 3, 1-3, 1-2, or 2-3 R^{a}.

Variable R¹

In some embodiments, R¹ can be:

(1-i) hydrogen; or

(1-ii) C₁-C₂₀ (e.g., C₁-C₁₂, C₁-C₆ or C₁-C₃) alkyl or C₁-C₂₀ (e.g., C₁-C₁₂, C₁-C₆ or C₁-C₃) haloalkyl, each of which is optionally substituted with from 1-10 (e.g., 1-5, 1-4, 1-3, 1-2, 1) R^{a}; or

(1-iv) C₃-C₂₀ (e.g., C₃-C₁₂, C₃-C₁₀, C₃-C₈, or C₃-C₆) cycloalkyl, C₃-C₂₀ (e.g., C₃-C₁₂, C₃-C₁₀, C₃-C₈, or C₃-C₆) cycloalkenyl, heterocyclyl including 3-20 (e.g., 3-12, 3-10, 3-8, or 3-6) atoms, heterocycloalkenyl including 3-20 (e.g., 3-12, 3-10, 3-8, or 3-6) atoms, C₇-C₂₀ (e.g., C₇-C₁₆, C₇-C₁₂, C₇-C₁₀) aralkyl, or heteroaralkyl including 6-20 (e.g., 6-14, 6-12, 6-10) atoms, each of which is optionally substituted with from 1-10 (e.g., 1-5, 1-4, 1-3, 1-2, 1) R^{c}; or

(1-v) C₆-C₁₈ (e.g., C₆-C₁₄, C₆-C₁₀, phenyl) aryl or heteroaryl including 5-16 (e.g., 5-14, 5-10, 5-6) atoms, each of which is optionally substituted with from 1-10 (e.g., 1-5, 1-4, 1-3, 1-2, 1) R^{d}.

In some embodiments, R¹ can be:

(1-i) hydrogen; or

(1-ii) C₁-C₂₀ (e.g., C₁-C₁₂, C₁-C₆ or C₁-C₃) alkyl or C₁-C₂₀ (e.g., C₁-C₁₂, C₁-C₆ or C₁-C₃) haloalkyl, each of which is optionally substituted with from 1-10 (e.g., 1-5, 1-4, 1-3, 1-2, 1) R^{a}; or

(1-iv′) C₃-C₂₀ (e.g., C₃-C₁₂, C₃-C₁₀, C₃-C₈, or C₃-C₆) cycloalkyl, C₃-C₂₀ (e.g., C₃-C₁₂, C₃-C₁₀, C₃-C₈, or C₃-C₆) cycloalkenyl, heterocyclyl including 3-20 (e.g., 3-12, 3-10, 3-8, or 3-6) atoms, or heterocycloalkenyl including 3-20 (e.g., 3-12, 3-10, 3-8, or 3-6) atoms, each of which is optionally substituted with from 1-10 (e.g., 1-5, 1-4, 1-3, 1-2, 1) R^{c}; or

(1-v) C₆-C₁₈ (e.g., C₆-C₁₄, C₆-C₁₀, phenyl) aryl or heteroaryl including 5-16 (e.g., 5-14, 5-10, 5-6) atoms, each of which is optionally substituted with from 1-10 (e.g., 1-5, 1-4, 1-3, 1-2, 1) R^{d}.

In some embodiments, R¹ can be any one of: (1-i), (1-ii), (1-iv), (1-iv′), and (1-v). In certain embodiments, R¹ can be hydrogen. In other embodiments, R¹ can be a substituent other than hydrogen.

In some embodiments, R¹ can be any two of (1-i), (1-ii), (1-iv), (1-iv′), and (1-v). In certain embodiments, R¹ can be hydrogen and any one of (1-ii), (1-iv), (1-iv′), and (1-v). In other embodiments, R¹ can be any two of (1-ii), (1-iv), (1-iv′), and (1-v), e.g., R¹ can be (1-ii) and (1-v).

In some embodiments, R¹ can be any three of: (1-i), (1-ii), (1-iv), (1-iv′), and (1-v). In certain embodiments, R¹ can be hydrogen and any two of (1-ii), (1-iv), (1-iv′), and (1-v), e.g., R¹ can be (1-iii) and (1-v). In other embodiments, R¹ can be any three of (1-ii), (1-iv), (1-iv′), and (1-v), e.g., (1-ii), (1-iv′), and (1-v).

In embodiments, R¹ can be C₁-C₆ (e.g., C₁-C₃) alkyl. For example, R¹ can be CH₃.

In certain embodiments, when R¹ is alkyl that is substituted with one or more R^{a}, then R^{a} can be other than NR^{m}Rⁿ; —NR^{o}C(O)R^{k}; —NR^{o}C(O)NR^{m}Rⁿ; —NR^{o}C(O)OR^{k}; —NR^{o}S(O)ₙR^{q}; optionally substituted heterocyclyl including 3-20 atoms; and optionally substituted heterocycloalkenyl including 3-20 atoms. In certain embodiments, R^{a} can be other than NR^{m}Rⁿ; optionally substituted heterocyclyl including 3-20 atoms; and optionally substituted heterocycloalkenyl including 3-20 atoms.

In embodiments, R¹ can be C₁-C₆ (e.g., C₁-C₃) haloalkyl (e.g., perhaloalkyl). For example, R¹ can be CF₃.

In embodiments, R¹ can be C₆-C₁₀ aryl, which is optionally substituted with from 1-5 (e.g., 1-4, 1-3, 1-2, 1) R^{d}. For example, R¹ can be phenyl, which is optionally substituted with from 1-5 (e.g., 1-4, 1-3, 1-2, 1) R^{d}.

In embodiments, R¹ can be heteroaryl including 5-10 (e.g., 5-6) atoms, each of which is optionally substituted with from 1-5 (e.g., 1-4, 1-3, 1-2, 1) R^{d}. For example, R¹ can be thienyl, furyl, pyrrolyl, or pyridinyl, each of which is optionally substituted with from 1-5 (e.g., 1-4, 1-3, 1-2, 1) R^{d}.

In certain embodiments, when R¹ is aryl (e.g., phenyl) that is substituted with one or more R^{d}, then R^{d} is other than hydroxyl.

In certain embodiments, when R¹ is aryl or heteroaryl that is substituted with one or more R^{d}, then R^{d} is other than —C(O)R^{k}, —C(O)OR^{k}; —C(O)SR^{k}; —C(S)SR^{k}; —C(O)NR^{m}Rⁿ; —S(O)ₙR^{q}; and C₂-C₂₀ (e.g., C₂-C₁₂, C₂-C₁₀, C₂-C₈, or C₂-C₆) alkenyl, which is optionally substituted with from 1-10 R^{b}.

In embodiments, R¹ can be C₃-C₂₀ (e.g., C₃-C₁₂, C₃-C₁₀, C₃-C₈, or C₃-C₆) cycloalkyl or C₃-C₂₀ (e.g., C₃-C₁₂, C₃-C₁₀, C₃-C₈, or C₃-C₆) cycloalkenyl, each of which is optionally substituted with from 1-3 R^{c}.

In embodiments, R¹ can be heterocyclyl including 3-20 (e.g., 3-12, 3-10, 3-8, or 3-6) atoms or heterocycloalkenyl including 3-20 (e.g., 3-12, 3-10, 3-8, or 3-6) atoms, each of which is optionally substituted with from 1-10 (e.g., 1-5, 1-4, 1-3, 1-2, 1) R^{c}.

In embodiments, R¹ can be C₃-C₂₀ (e.g., C₃-C₁₂, C₃-C₁₀, C₃-C₈, or C₃-C₆) cycloalkyl or heterocyclyl including 3-20 (e.g., 3-12, 3-10, 3-8, or 3-6) atoms, each of which is optionally substituted with from 1-10 (e.g., 1-5, 1-4, 1-3, 1-2, 1) R^{c}.

In embodiments, R¹ can be C₃-C₈ (e.g., C₃-C₇ or C₃-C₆) cycloalkyl, which is optionally substituted with from 1-3 R^{c}.

In embodiments, R¹ can be heterocyclyl including 3-8 (e.g., 3-7 or 3-6) atoms, which is optionally substituted with from 1-3 R^{c}.

Variable R²

In some embodiments, R² can be C₆-C₁₈ (e.g., C₆-C₁₄, C₆-C₁₀, phenyl) aryl, which is (i) substituted with from 1-5 (e.g., 1-4, 1-3, 1-2, 1) R⁷ and (ii) optionally substituted with from 1-4 (e.g., 1-3, 1-2, 1) R^{e}.

In some embodiments, when R² is aryl and substituted with R^{e}, each R^{e} can be independently of one another: halo (e.g., chloro); C₁-C₃ alkyl, optionally substituted with from 1-3 R^{a} (e.g., hydroxyl or —C(O)OR^{k}, e.g., the alkyl group can be CH₂C(O)OR^{k}); C₁-C₃ haloalkyl (e.g., C₁-C₃ fluoroalkyl, e.g., 1-5 fluorines can be present; or C₁-C₃ perfluoroalkyl); CN; mercapto; C₁-C₆ thioalkoxy optionally substituted with from 1-3 R^{a}; C₆-C₁₀ aryl (e.g., phenyl) or C₆-C₁₀ aryloxy (e.g., phenoxy), each of which is optionally substituted with from 1-10 R^{d}; hydroxyl; NR^{m}Rⁿ (e.g., NH₂, monoalkylamino, or dialkylamino); nitro; C₂-C₄ alkenyl; C₂-C₄ alkynyl; C₁-C₃ alkoxy; C₁-C₃ haloalkoxy; —C(O)OR^{k} (e.g., R^{k} can be hydrogen or C₁-C₃ alkyl); or —C(O)R^{k} (e.g., R^{k} can be C₁-C₃ alkyl).

In certain embodiments, when R² is substituted with R^{e}, each R^{e} can be independently of one another: C₁-C₃ alkyl; C₁-C₃ haloalkyl, e.g., C₁-C₃ perfluoroalkyl; halo (e.g., chloro or fluoro, e.g., chloro); or CN.

In certain embodiments, when R² is substituted with R^{e}, each R^{e} can be independently of one another: C₁-C₃ alkyl; C₁-C₃ haloalkyl, e.g., C₁-C₃ perfluoroalkyl; halo (e.g., chloro or fluoro, e.g., chloro).

In certain embodiments, when R² is substituted with R^{e}, each R^{e} can be independently of one another halo (e.g., chloro or fluoro, e.g., chloro).

In some embodiments, R² can be C₆-C₁₀ aryl, which is (i) substituted with from 1-5 (e.g., 1-4, 1-3, 1-2, 1) R⁷ and (ii) optionally substituted with from 1-4 (e.g., 1-3, 1-2, 1) R^{e}.

In some embodiments, R² can be C₆-C₁₀ aryl, which is (i) substituted with 1 or 2 R⁷ and (ii) optionally substituted with 1 or 2 R^{e}.

In some embodiments, R² can be phenyl, which is (i) substituted with 1 or 2 R⁷ and (ii) optionally substituted with 1 or 2 R^{e} (e.g., e.g., halo, e.g., chloro or fluoro, e.g., chloro).

In certain embodiments, R² can be phenyl, which is (i) substituted with 1 R⁷ and (ii) optionally substituted with 1 or 2 (e.g., 1) R^{e} (e.g., halo, e.g., chloro or fluoro, e.g., chloro). In other embodiments, R² can be phenyl, which is substituted with 1 R⁷. In these embodiments, R² can have formula (A), in which R⁷ (i.e., the moiety —WA) can be attached to a ring carbon that is ortho, meta, or para (e.g., meta) with respect to the ring carbon that connects the phenyl ring to the 4-position of the quinazoline ring, and R^{e}, when present can be connected to ring carbons that are not occupied by WA. For example, R² can have formula (A-1), in which R⁷ (WA) is attached to the ring carbon that is meta with respect to the ring carbon that connects the phenyl ring to the 4-position of the quinoline ring in formula (I).

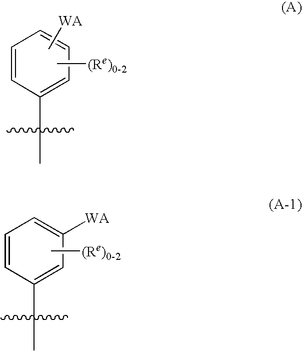

In certain embodiments, R² can have formula (A-2):

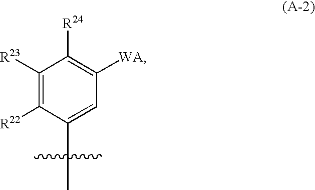

in which each of R²², R²³, and R²⁴ can be, independently of one another, hydrogen or R^{e}, in which R^{e} can be as defined anywhere herein.

In embodiments, each of R²², R²³, and R²⁴ can be hydrogen. In other embodiments, each of R²², R²³, and R²⁴ can be a substituent other than hydrogen. In still other embodiments, one or two of R²², R²³, and R²⁴ can be R^{e}, and the other(s) are hydrogen.

In certain embodiments, one of R²², R²³, and R²⁴ can be R^{e}, and the other two are hydrogen. In embodiments, R²² can be R^{e}, and each of R²³ and R²⁴ can be hydrogen. In certain embodiments, R^{e} can be: halo (e.g., chloro or fluoro, e.g., chloro); C₁-C₃ alkyl, optionally substituted with from 1-3 R^{a}; or C₁-C₃ haloalkyl (e.g., C₁-C₃ fluoroalkyl, e.g., 1-5 fluorines can be present; or C₁-C₃ perfluoroalkyl). In certain embodiments, R^{e} can be halo (e.g., chloro).

In some embodiments, R² can be heteroaryl including 5-16 (e.g., 5-14, 5-10, 5-6) atoms, which is (i) substituted with from 1-5 (e.g., 1-4, 1-3, 1-2, 1) R⁷ and (ii) optionally substituted with from 1-4 (e.g., 1-3, 1-2, 1) R^{e}.

In embodiments, when R² is heteroaryl and substituted with R^{e}, each R^{e} can be independently as defined anywhere herein. For example, each R^{e} can be independently of one another: C₁-C₃ alkyl; C₁-C₃ haloalkyl, e.g., C₁-C₃ perfluoroalkyl; halo (e.g., chloro); e.g., each R^{e} can be halo (e.g., chloro).

In some embodiments, R² can be heteroaryl including 5-12 (e.g., 5-10) atoms, which is (i) substituted with from 1-4 (e.g., 1-3, 1-2, 1) R⁷ and (ii) optionally substituted with from 1-4 (e.g., 1-3, 1-2, 1) R^{e}.

In some embodiments, R² can be heteroaryl including 5-12 (e.g., 5-10) atoms, which is (i) substituted 1 or 2 R⁷ and (ii) optionally substituted with 1 or 2 R^{e}.

In some embodiments, R² can be heteroaryl including 5-6 atoms, which is (i) substituted 1 or 2 R⁷ and (ii) optionally substituted with 1 or 2 R^{e}.

In some embodiments, R² can be heteroaryl including 8-10 atoms, which is (i) substituted 1 or 2 R⁷ and (ii) optionally substituted with 1 or 2 R^{e}.

In certain embodiments, R² can be pyridyl, pyrimidinyl, thienyl, furyl, quinolinyl, oxazolyl, thiazolyl, imidazolyl, isoxazolyl, indolyl, benzo[1,3]-dioxolyl, benzo[1,2,5]-oxadiazolyl, isochromenyl-1-one, 3-H-isobenzofuranyl-1-one (e.g., pyridyl, thienyl, or indolyl, e.g., pyridyl), each of which is (i) substituted with 1 R⁷ and (ii) optionally substituted with 1 or 2 R^{e}. For example, R² can be pyridyl substituted with 1 R⁷.

Variable W

In some embodiments, W can be —O—.

In some embodiments, W can be a bond.

In other embodiments, W can be —W¹(C₁₋₆ alkylene)-. In certain embodiments, W¹ can be —O—. For example, W can be —O(C₁₋₃ alkylene)- (e.g., —OCH₂—).

In some embodiments, W can be —NR⁸— (e.g., —NH—).

In some embodiments, W can be —(C₁₋₆ alkylene)W¹—. In certain embodiments, W¹ is —NR⁹—, in which R⁹ can be hydrogen; or W¹ can be —O—. In certain embodiments, W can be —(C₁₋₃ alkylene)NH— (e.g., —CH₂NH—). In certain embodiments, W can be —(C₁₋₃ alkylene)O— (e.g., —CH₂O—).

In still other embodiments, W can be C₂-C₄ alkenylene (e.g., —CH═CH—); C₂-C₄ alkynylene (e.g., —C [symbol] —); or C₁₋₃ alkylene (e.g., CH₂).

Variable A

In general, A is an aromatic or heteroaromatic ring system that is (a) substituted with one or more R⁹; and (b) optionally substituted with one or more R^{g}.

In some embodiments, A can be C₆-C₁₀ (e.g., phenyl) aryl, which is (a) substituted with from 1-5 (e.g., 1-4, 1-3, 1-2, 1, e.g., 1) R⁹; and (b) optionally further substituted with from 1-6 (e.g., 1-5, 1-4, 1-3, 1-2, 1, e.g., 1-2) R^{g}, in which R^{g} can be as defined anywhere herein.

In embodiments, when A is aryl and substituted with one or more R^{g}, each R^{g} can be independently of one another:

(i) halo; C₁-C₁₂ (e.g., C₁-C₆, C₁-C₃) alkoxy or C₁-C₁₂ (e.g., C₁-C₆, C₁-C₃) haloalkoxy, each of which is optionally substituted with from 1-5 (e.g., 1-4, 1-3, 1-2, or 1) R^{a}; C₆-C₁₀ aryloxy or heteroaryloxy including 5-10 atoms, each of which is optionally substituted with from 1-5 (e.g., 1-4, 1-3, 1-2, or 1) R^{d}; C₇-C₁₂ aralkoxy, heteroaralkoxy including 6-12 atoms, C₃-C₁₀ (e.g., C₃-C₆) cycloalkoxy, C₃-C₁₀ (e.g., C₃-C₆) cycloalkenyloxy, heterocyclyloxy including 3-10 (e.g., 3-6) atoms, or heterocycloalkenyloxy including 3-10 (e.g., 3-6) atoms, each of which is optionally substituted with from 1-5 (e.g., 1-4, 1-3, 1-2, or 1) R^{c}; mercapto; C₁-C₁₂ (e.g., C₁-C₆, C₁-C₃) thioalkoxy; C₁-C₁₂ (e.g., C₁-C₆, C₁-C₃) thiohaloalkoxy; C₆-C₁₀ thioaryloxy or thioheteroaryloxy including 5-10 atoms, each of which is optionally substituted with from 1-5 (e.g., 1-4, 1-3, 1-2, or 1) R^{d}; C₇-C₁₂ thioaralkoxy, thioheteroaralkoxy including 6-12 atoms, C₃-C₁₀ (e.g., C₃-C₆) thiocycloalkoxy, C₃-C₁₀ (e.g., C₃-C₆) thiocycloalkenyloxy, thioheterocyclyloxy including 3-10 (e.g., 3-6) atoms, or thioheterocycloalkenyloxy including 3-10 (e.g., 3-6) atoms, each of which is optionally substituted with from 1-5 (e.g., 1-4, 1-3, 1-2, or 1) R^{c}; or cyano; or

(ii) C₁-C₁₂ (e.g., C₁-C₆, C₁-C₃) alkyl or C₁-C₁₂ (e.g., C₁-C₆, C₁-C₃) haloalkyl, each of which is optionally substituted with from 1-5 (e.g., 1-4, 1-3, 1-2, or 1) R^{a}; or

(iii) C₂-C₁₂ (e.g., C₂-C₈, C₂-C₄) alkenyl or C₂-C₁₂ (e.g., C₂-C₈, C₂-C₄) alkynyl, each of which is optionally substituted with from 1-5 (e.g., 1-4, 1-3, 1-2, or 1) R^{b}.

In embodiments, when A is aryl and substituted with one or more R^{g}, each R^{g} can be independently of one another:

(i) halo; C₁-C₁₂ (e.g., C₁-C₆, C₁-C₃) alkoxy or C₁-C₁₂ (e.g., C₁-C₆, C₁-C₃) haloalkoxy, each of which is optionally substituted with from 1-5 (e.g., 1-4, 1-3, 1-2, or 1) R^{a}; C₆-C₁₀ aryloxy or heteroaryloxy including 5-10 atoms, each of which is optionally substituted with from 1-5 (e.g., 1-4, 1-3, 1-2, or 1) R^{d}; C₇-C₁₂ aralkoxy, heteroaralkoxy including 6-12 atoms, C₃-C₁₀ (e.g., C₃-C₆) cycloalkoxy, C₃-C₁₀ (e.g., C₃-C₆) cycloalkenyloxy, heterocyclyloxy including 3-10 (e.g., 3-6) atoms, or heterocycloalkenyloxy including 3-10 (e.g., 3-6) atoms, each of which is optionally substituted with from 1-5 (e.g., 1-4, 1-3, 1-2, or 1) R^{c}; or cyano; or

(ii) C₁-C₁₂ (e.g., C₁-C₆, C₁-C₃) alkyl or C₁-C₁₂ (e.g., C₁-C₆, C₁-C₃) haloalkyl, each of which is optionally substituted with from 1-5 (e.g., 1-4, 1-3, 1-2, or 1) R^{a}.

In embodiments, when A is aryl and substituted with one or more R^{g}, each R^{g} can be independently of one another:
halo (e.g., chloro or fluoro); or
C₁-C₁₂ (e.g., C₁-C₆, C₁-C₃) alkoxy which is optionally substituted with from 1-5 (e.g., 1-4, 1-3, 1-2, or 1) R^{a} (e.g., R^{a} can be hydroxyl; C₁-C₃ alkoxy; C₃-C₇ cycloalkoxy or aryloxy, each of which can be optionally substituted with R^{c} and R^{d}, respectively; NR^{m}Rⁿ; or heterocyclyl including 3-8 atoms, which is optionally substituted with from 1-5 R^{c});
C₁-C₁₂ (e.g., C₁-C₆, C₁-C₃) haloalkoxy; or
C₆-C₁₀ aryloxy or heteroaryloxy including 5-10 atoms, each of which is optionally substituted with from 1-5 (e.g., 1-4, 1-3, 1-2, or 1) R^{d}; or
C₇-C₁₂ aralkoxy, which is optionally substituted with from 1-5 (e.g., 1-4, 1-3, 1-2, or 1) R^{c}; or
cyano; or
C₁-C₁₂ (e.g., C₁-C₆, C₁-C₃) alkyl or C₁-C₁₂ (e.g., C₁-C₆, C₁-C₃) haloalkyl, each of which is optionally substituted with from 1-5 (e.g., 1-4, 1-3, 1-2, or 1) R^{a}.

In some embodiments, A can be C₆-C₁₀ aryl, which is (i) substituted with 1 or 2 R⁹ and (ii) optionally substituted with from 1-6 (e.g., 1-5, 1-4, 1-3, 1-2, 1, e.g., 1-2) R^{g}.

In some embodiments, A can be C₆-C₁₀ aryl, which is (i) substituted with 1 R⁹ and (ii) optionally substituted with from 1-6 (e.g., 1-5, 1-4, 1-3, 1-2, 1, e.g., 1-2) R^{g}.

In some embodiments, A can be phenyl, which is (i) substituted with 1 R⁹ and (ii) optionally substituted with from 1-4 (e.g., 1-3, 1-2, 1) R^{g}.

In these embodiments, R⁹ can be attached to a ring carbon that is ortho, meta, or para (e.g., meta or para) with respect to the ring carbon that connects the phenyl ring to W.

In certain embodiments, A can have formula (B-1):

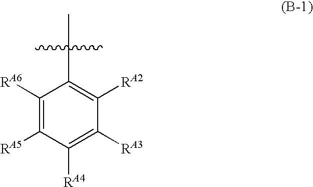

in which one of R^{A3} and R^{A4} is R⁹, the other of R^{A3} and R^{A4} and each of R^{A2}, R^{A5}, and R^{A6} is, independently, hydrogen or R^{g}, in which R^{g} can be as defined anywhere herein.

In embodiments, one of R^{A3} and R^{A4} can be R⁹, the other of R^{A3} and R^{A4} can be hydrogen; and each of R^{A2}, R^{A5}, and R^{A6} can be, independently, hydrogen or R^{g}.

In certain embodiments, R^{A3} can be R⁹, R^{A4} can be hydrogen, and each of R^{A2}, R^{A5}, and R^{A6} can be hydrogen. In other embodiments, R^{A3} can be R⁹; R^{A4} can be hydrogen; one of R^{A2}, R^{A5}, and R^{A6} (e.g., R^{A5}) can be R^{g} (e.g., halo) and the other two of R^{A2}, R^{A5}, and R^{A6} can be hydrogen.

In certain embodiments, R^{A4} can be R⁹, R^{A3} can be hydrogen, and each of R^{A2}, R^{A5}, and R^{A6} can be hydrogen. In other embodiments, R^{A3} can be R⁹; R^{A4} can be hydrogen; one of R^{A2}, R^{A5}, and R^{A6} can be R^{g} (e.g., halo) and the other two of R^{A2}, R^{A5}, and R^{A6} can be hydrogen.

In some embodiments, A can be heteroaryl including 5-10 atoms, which is (a) substituted with from 1-3 (e.g., 1-2, 1) R⁹; and (b) is optionally substituted with from 1-3 (e.g., 1-2, 1) R^{g}, in which R^{g} can be as defined anywhere herein.

In some embodiments, A can be heteroaryl including 5-10 atoms, which is (a) substituted with 1 R⁹; and (b) is optionally substituted with from 1-3 (e.g., 1-2, 1) R^{g}.

In certain embodiments, A can be pyrrolyl, pyridyl, pyridyl-N-oxide, pyrazolyl, pyrimidinyl, thienyl, furyl, quinolinyl, oxazolyl, thiazolyl, imidazolyl, isoxazolyl, indolyl, benzo[1,3]-dioxolyl, benzo[1,2,5]-oxadiazolyl, isochromenyl-1-one, 3-H-isobenzofuranyl-1-one (e.g., pyridyl, thienyl, or indolyl, e.g., pyridyl), which is (i) substituted with 1 R⁹ and (ii) optionally substituted with 1-3 (e.g., 1-2, 1) R^{g}.

In certain embodiments, A can be pyrrolyl, pyridyl, pyrimidinyl, pyrazolyl, thienyl, furyl, quinolyl, oxazolyl, thiazolyl, imidazolyl, or isoxazolyl, each of which is (a) substituted with 1 R⁹; and (b) is optionally substituted with from 1-3 (e.g., 1-2, 1) R^{g}.

In certain embodiments, A can be pyridyl, pyrimidinyl, thienyl, furyl, oxazolyl, thiazolyl, imidazolyl, or isoxazolyl, each of which is (a) substituted with 1 R⁹; and (b) is optionally substituted with from 1-3 (e.g., 1-2, 1) R^{g}.

In certain embodiments, A can be pyridyl in which W is attached to the 2- or 3-position of the pyridiyl ring. For example, A can be pyridyl in which W is attached to the 2-position of the pyridyl ring, and R⁹ is attached to the 4- or the 6-position of the pyridyl ring. Such rings can be further substituted with 1, 2 or 3 R^{g} (e.g., halo, e.g., chloro; or NR^{g}R^{h}, e.g., NH₂).

Variable R⁹

R⁹ can be:

(9-i) —W²—S(O)ₙR¹⁰ or —W²—S(O)ₙNR¹¹R¹²; or

(9-ii) —W²—C(O)OR¹³; or

(9-iii) —W²—C(O)NR¹¹R¹²; or

(9-iv) —W²—CN; or

(9-v) C₁-C₁₂ alkyl or C₁-C₁₂ haloalkyl, each of which is:
(a) substituted with from 1-3 R^{h}, and
(b) optionally further substituted with from 1-5 R^{a};

or

(9-vi) C₇-C₂₀ aralkyl or heteroaralkyl including 6-20 atoms, each of which is:
(a) substituted with from 1-3 R^{h}, and
(b) optionally further substituted with from 1-5 substituents independently selected from R^{c}; C₁-C₆ alkyl, which is optionally substituted with from 1-3 R^{a}; C₁-C₆ haloalkyl; C₆-C₁₀ aryl, which is optionally substituted with from 1-10 R^{d}; halo; C₂-C₆ alkenyl; or C₂-C₆ alkynyl;

or

(9-vii) —NR¹⁴R¹⁵.

In some embodiments, R⁹ can be:
(9-i′) —W²—S(O)ₙR¹⁰; or
(9-ii), (9-iii), (9-iv), (9-v), (9-vi), or (9-vii).

In some embodiments, R⁹ can be any one of: (9-i), (9-i′), (9-ii), (9-iii), (9-iv), (9-v), (9-vi), or (9-vii). In certain embodiments, R⁹ can be —W²—S(O)ₙR¹⁰ or —W²—S(O)ₙNR¹¹R¹² (e.g., —W²—S(O)ₙR¹⁰). In other embodiments, R⁹ can be —W²—C(O)OR¹³.

In some embodiments, R⁹ can be any two of: (9-i), (9-i′), (9-ii), (9-iii), (9-iv), (9-v), (9-vi), or (9-vii). In certain embodiments, R⁹ can be —W²—S(O)ₙR¹⁰ or —W²—S(O)ₙNR¹¹R¹² (e.g., —W²—S(O)ₙR¹⁰) and any one of (9-ii), (9-iii), (9-iv), (9-v), (9-vi), or (9-vii). For example, R⁹ can be:
—W²—S(O)ₙR¹⁰ or —W²—S(O)ₙNR¹¹R¹² (e.g., —W²—S(O)ₙR¹⁰); and
—W²—C(O)OR¹³.

In other embodiments, R⁹ can be any two of (9-ii), (9-iii), (9-iv), (9-v), (9-vi), or (9-vii).

In some embodiments, R⁹ can be any three of: (9-i), (9-i′), (9-ii), (9-iii), (9-iv), (9-v), (9-vi), or (9-vii). In certain embodiments, R⁹ can be:
—W²—S(O)ₙR¹⁰ or —W²—S(O)ₙNR¹¹R¹² (e.g., —W²—S(O)ₙR¹⁰); and
—W²—C(O)OR¹³; and
any one of (9-iii), (9-iv), (9-v), (9-vi), or (9-vii).

In other embodiments, R⁹ can be any three of (9-iii), (9-iv), (9-v), (9-vi), or (9-vii).

In some embodiments, R⁹ can be —W²—S(O)ₙR¹⁰ (e.g., —W²—S(O)₂R¹⁰, i.e., n is 2).

In some embodiments, R¹⁰ can be C₁-C₁₀ (e.g., C₁-C₆ or C₁-C₃) alkyl or C₁-C₁₀ (e.g., C₁-C₆ or C₁-C₃) haloalkyl, optionally substituted with from 1-2 R^{a}. For example, R¹⁰ can be C₁-C₁₀ (e.g., C₁-C₆ or C₁-C₃) alkyl, optionally substituted with from 1-2 R^{a}.

In certain embodiments, R¹⁰ can be CH₃.

In certain embodiments, R¹⁰ can be C₁-C₃ or C₂-C₈ alkyl, which is optionally substituted with 1 R^{a}. For example, R¹⁰ can be CH₃, CH₂CH₃, or (CH₃)₂CH. As another example, R¹⁰ can be unsubstituted branched or unbranched C₃-C₈ alkyl.

In certain embodiments, R¹⁰ can be branched or unbranched C₂-C₈ (e.g., C₃-C₈) alkyl, which is substituted with 1 R^{a}. In embodiments, R^{a} can be: hydroxyl; C₁-C₆ (e.g., C₁-C₃) alkoxy; C₃-C₇ cycloalkoxy or C₆-C₁₀ aryloxy, each of which can be optionally substituted with R^{c} and R^{d}, respectively; NR^{m}Rⁿ; or heterocyclyl including 3-8 atoms, which is optionally substituted with from 1-5 R^{c}. For example, R^{a} can be hydroxyl or C₁-C₆ (e.g., C₁-C₃) alkoxy. In certain embodiments, R^{a} can be attached to a secondary or tertiary carbon atom of the alkyl group. In other embodiments, R^{a} can be attached to a primary (terminal) carbon atom of the alkyl group.

In certain embodiments, R¹⁰ can be C₇-C₁₂ aralkyl (e.g., benzyl), optionally substituted with from 1-3 (e.g., 1-2, 1) R^{c}.

In certain embodiments, R¹⁰ can be C₆-C₁₀ aryl, optionally substituted with from 1-2 R^{d}.

In certain embodiments, W² can be a bond. In other embodiments, W² can be C₁₋₆ alkylene (e.g., CH₂).

In some embodiments, R⁹ can be —W²—S(O)ₙNR¹¹R¹² (e.g., —W²—S(O)₂NR¹¹R¹²).

In certain embodiments, R¹¹ and R¹² can each be, independently of one another:

(i) C₁-C₂₀ alkyl or C₁-C₂₀ haloalkyl, each of which is optionally substituted with from 1-10 R^{a}; or

(ii) C₂-C₂₀ alkenyl or C₂-C₂₀ alkynyl, each of which is optionally substituted with from 1-10 R^{b}; or

(iii) C₃-C₂₀ cycloalkyl, C₃-C₂₀ cycloalkenyl, heterocyclyl including 3-20 atoms, heterocycloalkenyl including 3-20 atoms, C₇-C₂₀ aralkyl, or heteroaralkyl including 6-20 atoms, each of which is optionally substituted with from 1-10 R^{c}; or

(iv) C₆-C₁₈ aryl or heteroaryl including 5-16 atoms, each of which is optionally substituted with from 1-10 R^{d}.

In certain embodiments, R¹¹ and R¹² can each be, independently of one another:

(i) C₁-C₁₂ (e.g., C₁-C₆ or C₁-C₃) alkyl or C₁-C₁₂ (e.g., C₁-C₆ or C₁-C₃) haloalkyl, each of which is optionally substituted with from 1-6 (e.g., 1-5, 1-4, 1-3, 1-2, 1) R^{a} (e.g., R^{a} can be: hydroxyl; C₁-C₆ (e.g., C₁-C₃) alkoxy; C₃-C₇ cycloalkoxy or C₆-C₁₀ aryloxy, each of which can be optionally substituted with R^{c} and R^{d}, respectively; NR^{m}Rⁿ; or heterocyclyl including 3-8 atoms, which is optionally substituted with from 1-5 R^{c}); or

(iii) C₇-C₁₂ aralkyl, or heteroaralkyl including 6-12 atoms, each of which is optionally substituted with from 1-6 (e.g., 1-5, 1-4, 1-3, 1-2, 1) R^{c}; or

(iv) C₆-C₁₀ aryl or heteroaryl including 5-10 atoms, each of which is optionally substituted with from 1-6 (e.g., 1-5, 1-4, 1-3, 1-2, 1) R^{d}.

In certain embodiments, R¹¹ and R¹² together with the nitrogen atom to which they are attached can form a heterocyclyl including 3-20 (e.g., 3-10, 3-8, or 3-6) atoms or a heterocycloalkenyl including 3-20 (e.g., 3-10, 3-8, or 3-6) atoms, each of which is optionally substituted with from 1-5 (1-4, 1-3, 1-2, 1) R^{c}.

In certain embodiments, R¹¹ and R¹² together with the nitrogen atom to which they are attached can form a heterocyclyl including 3-10 (e.g., 3-8, 3-6, or 5-6) atoms, which is optionally substituted with from 1-5 (1-4, 1-3, 1-2, 1) R^{c}. For example, R¹¹ and R¹² together with the nitrogen atom to which they are attached can form a morpholinyl, piperidyl, pyrrolidinyl, or piperazinyl ring, each of which is optionally substituted with from 1-5 (1-4, 1-3, 1-2, 1) R^{c}.

In certain embodiments, one of R¹¹ and R¹² can be hydrogen, and the other of R¹¹ and R¹² can be:

(i) C₁-C₁₂ (e.g., C₁-C₆ or C₁-C₃) alkyl or C₁-C₁₂ (e.g., C₁-C₆ or C₁-C₃) haloalkyl, each of which is optionally substituted with from 1-6 (e.g., 1-5, 1-4, 1-3, 1-2, 1) R^{a} (e.g., R^{a} can be: hydroxyl; C₁-C₆ (e.g., C₁-C₃) alkoxy; C₃-C₇ cycloalkoxy or C₆-C₁₀ aryloxy, each of which can be optionally substituted with R^{c} and R^{d}, respectively; NR^{m}Rⁿ; or heterocyclyl including 3-8 atoms, which is optionally substituted with from 1-5 R^{c}); or

(iii) C₇-C₁₂ aralkyl, or heteroaralkyl including 6-12 atoms, each of which is optionally substituted with from 1-6 (e.g., 1-5, 1-4, 1-3, 1-2, 1) R^{c}; or

(iv) C₆-C₁₀ aryl or heteroaryl including 5-10 atoms, each of which is optionally substituted with from 1-6 (e.g., 1-5, 1-4, 1-3, 1-2, 1) R^{d}.

In certain embodiments, W² can be a bond.

In some embodiments, R⁹ can be —W²—C(O)OR¹³.

In some embodiments, R¹³ can be:

(i) hydrogen; or

C₁-C₁₀ (e.g., C₁-C₇) alkyl, which is optionally substituted with from 1-3 (e.g., 1-2, 1) R^{a}; or

(iii) C₃-C₇ cycloalkyl or C₇-C₁₂ aralkyl, each of which is optionally substituted with from 1-10 R^{c}; or

(iv) C₆-C₁₀ aryl or heteroaryl including 5-10 atoms, each of which is optionally substituted with from 1-10 R^{d}.

In certain embodiments, R¹³ can be hydrogen.

In some embodiments, W² can be C₁-C₆ alkylene, optionally substituted with from 1-3 R^{f}; or a bond.

In certain embodiments, W² can be C₁-C₆ alkylene. For example, W² can be C₁-C₃ alkylene, such as CH₂ or CH₂CH₂.

In certain embodiments, W² can be a bond.

In some embodiments, R⁹ can be —W²—C(O)NR¹¹R¹².

Embodiments can include, for example, any one or more of the features described above in conjunction with —W²—S(O)ₙNR¹¹R¹².

In some embodiments, R⁹ can be —W²—CN.

In some embodiments, W² can be C₁-C₆ alkylene, optionally substituted with from 1-3 R^{f}; or a bond.

In certain embodiments, W² can be C₁-C₆ alkylene. For example, W² can be C₁-C₃ alkylene, such as CH₂ or CH₂CH₂.

In certain embodiments, W² can be a bond.

In some embodiments, R⁹ can be:
C₁-C₁₂ alkyl or C₁-C₁₂ haloalkyl, each of which is (a) substituted with from 1 R^{h}, and (b) optionally further substituted with from 1 or 2 R^{a} (e.g., R^{a} can be C₃-C₇ cycloalkyl, which is optionally substituted with from 1-5 R^{c}); or
C₇-C₂₀ aralkyl or heteroaralkyl including 6-20 atoms, each of which is (a) substituted with from 1-3 R^{h}, and (b) optionally further substituted with from 1 or 2 substituents independently selected from C₃-C₇ cycloalkyl, which is optionally substituted with from 1-5 R^{c}; or C₆-C₁₀ aryl, which is optionally substituted with from 1-10 R^{d}.

In certain embodiments, R^{h} at each occurrence can be, independently, hydroxyl, C₁-C₁₂ alkoxy, C₁-C₁₂ haloalkoxy; C₃-C₁₀ cycloalkoxy, which is optionally substituted with from 1-5 R^{c}; or C₆-C₁₀ aryloxy or heteroaryloxy including 5-10 atoms, each of which is optionally substituted with from 1-5 R^{d}.

In certain embodiments, R⁹ can have the following formula: —C(R⁹¹)(R⁹²)(R^{h}), in which each of R⁹¹ and R⁹² is, independently, C₁-C₁₂ alkyl or C₁-C₁₂ haloalkyl, each of which is optionally further substituted with from 1 or 2 R^{a} (e.g., R^{a} can be C₃-C₇ cycloalkyl, which is optionally substituted with from 1-5 R^{c}); C₃-C₇ cycloalkyl, which is optionally substituted with from 1-5 R^{c}; or C₆-C₁₀ aryl, which is optionally substituted with from 1-10 R^{d}; and R^{h} can be as defined anywhere herein.

In some embodiments, R⁹ can be —NR¹⁴R¹⁵, one of R¹⁴ and R¹⁵ is hydrogen or C₁-C₃ alkyl (e.g., hydrogen); and the other of R¹⁴ and R¹⁵ can be:

(i) —S(O)ₙR¹⁰; or

(ii) —C(O)OR¹³; or

(iii) —C(O)NR¹¹R¹²; or

(iv) —CN; or

(v) C₁-C₁₂ alkyl or C₁-C₁₂ haloalkyl, each of which is:
(a) substituted with from 1-3 R^{h}, and
(b) optionally further substituted with from 1-5 R^{a};

or

(vi) C₇-C₂₀ aralkyl or heteroaralkyl including 6-20 atoms, each of which is:
(a) substituted with from 1-3 R^{h}, and
(b) optionally further substituted with from 1-5 substituents independently selected from R^{a}; C₁-C₆ alkyl, which is optionally substituted with from 1-3 R^{a}; C₁-C₆ haloalkyl; C₆-C₁₀ aryl, which is optionally substituted with from 1-10 R^{d}; halo; C₂-C₆ alkenyl; or C₂-C₆ alkynyl.

In embodiments, each of n, R¹⁰, R¹¹, R¹², R¹³, R^{h}, R^{a}, and R^{d} can be, independently, as defined anywhere herein.

Variables R³, R⁴, and R⁵

In some embodiments, each of R³, R⁴, and R⁵ can be, independently:

(i) hydrogen; or

(ii) halo; or

(iii) C₁-C₆ alkyl or C₁-C₆ haloalkyl, each of which is optionally substituted with from 1-3 R^{e}; or

(iv) C₃-C₆ cycloalkyl, which is optionally substituted with from 1-3 R^{c}; or

(v) C₆-C₁₀ aryl, which is optionally substituted with from 1-10 R^{d}.

In certain embodiments, each of R³, R⁴, and R⁵ can be, independently:

(i) hydrogen; or

(ii) halo; or

(iii) C₁-C₆ (e.g., C₁-C₃) alkyl or C₁-C₆ (e.g., C₁-C₃) haloalkyl (e.g., perhaloalkyl, e.g., perfluoroalkyl), each of which is optionally substituted with from 1-3 R^{e}.

In certain embodiments, each of R³, R⁴, and R⁵ can be, independently, hydrogen or halo (e.g., fluoro).

In certain embodiments, each of R³, R⁴, and R⁵ can be hydrogen.

In certain embodiments, each of R³, R⁴, and R⁵ can be a substituent other than hydrogen.

In certain embodiments, one or two of R³, R⁴, and R⁵ can be hydrogen, and the other(s) can be:

(ii) halo; or

(iii) C₁-C₆ (e.g., C₁-C₃) alkyl or C₁-C₆ (e.g., C₁-C₃) haloalkyl (e.g., perhaloalkyl, e.g., perfluoroalkyl), each of which is optionally substituted with from 1-3 R^{e}.

Variable R⁶

In some embodiments, R⁶ can be:

(i) halo (e.g., choro); or

(ii) C₁-C₆ alkyl or C₁-C₆ haloalkyl, each of which is optionally substituted with from 1-3 R^{a}; or

(iii) cyano; —C(O)NR^{m}Rⁿ; .—C(O)R^{k}; or —S(O)ₙR^{q}, wherein n is 1 or 2.

In certain embodiments, the definition of R⁶ can further include C₁-C₃ alkoxy.

In some embodiments, R⁶ can be halo, cyano, C₁-C₆ (e.g., C₁-C₃) alkyl, C₁-C₆ (e.g., C₁-C₃) haloalkyl, or SO₂R^{q}.

In some embodiments, R⁶ can be chloro or bromo (e.g., chloro); cyano, C₁-C₆ (e.g., C₁-C₃) alkyl, C₁-C₆ (e.g., C₁-C₃) haloalkyl, or SO₂R^{q}.

In some embodiments, R⁶ can be halo, C₁-C₆ (e.g., C₁-C₃) alkyl, C₁-C₆ (e.g., C₁-C₃) haloalkyl, or SO₂R^{q}.

In some embodiments, R⁶ can be chloro or bromo (e.g., chloro); C₁-C₆ (e.g., C₁-C₃) alkyl, C₁-C₆ (e.g., C₁-C₃) haloalkyl, or SO₂R^{q}.

In some embodiments, R⁶ can be halo, C₁-C₆ (e.g., C₁-C₃) alkyl, or C₁-C₆ (e.g., C₁-C₃) haloalkyl.

In some embodiments, R⁶ can be chloro or bromo (e.g., chloro), C₁-C₆ (e.g., C₁-C₃) alkyl, or C₁-C₆ (e.g., C₁-C₃) haloalkyl.

In some embodiments, R⁶ can be halo (e.g., chloro) or C₁-C₆ (e.g., C₁-C₃) haloalkyl (e.g., CF₃).

In some embodiments, R⁶ can be chloro or bromo (e.g., chloro) or C₁-C₆ (e.g., C₁-C₃) haloalkyl.

In certain embodiments, R⁶ can be chloro; cyano; CH₃; CF₃; or SO₂CH₃. In certain embodiments, R⁶ can be chloro; CH₃; or CF₃. In certain embodiments, R⁶ can be chloro or CF₃.

In some embodiments, R⁶ can be C₁-C₆ (e.g., C₁-C₃) haloalkyl (e.g., perfluoroalkyl, e.g., CF₃).

In some embodiments, R⁶ can be halo (e.g., chloro).

In some embodiments, R⁶ can be C₁-C₆ (e.g., C₁-C₃) alkyl (e.g., CH₃).

In some embodiments, R⁶ can be SO₂R^{q}.

In certain embodiments, R^{q} can be C₁-C₁₀ (e.g., C₁-C₆ or C₁-C₃) alkyl or C₁-C₁₀ (e.g., C₁-C₆ or C₁-C₃) haloalkyl, optionally substituted with from 1-2 R^{a}. For example, R^{q} can be CH₃.

In embodiments, R^{a} can be: hydroxyl; C₁-C₆ (e.g., C₁-C₃) alkoxy; C₃-C₇ cycloalkoxy or C₆-C₁₀ aryloxy, each of which can be optionally substituted with R^{c} and R^{d}, respectively; NR^{m}Rⁿ; or heterocyclyl including 3-8 atoms, which is optionally substituted with from 1-5 R^{c}.

In certain embodiments, R^{q} can be C₇-C₁₂ aralkyl (e.g., benzyl), optionally substituted with from 1-3 (e.g., 1-2, 1) R^{c}.

In certain embodiments, R^{q} can be C₆-C₁₀ aryl, optionally substituted with from 1-2 R^{d}.

In certain embodiments, R^{q} can be NR^{m}Rⁿ.

In embodiments, R^{m} and Rⁿ can each be, independently of one another:

(i) C₁-C₁₂ (e.g., C₁-C₆ or C₁-C₃) alkyl or C₁-C₁₂ (e.g., C₁-C₆ or C₁-C₃) haloalkyl, each of which is optionally substituted with from 1-6 (e.g., 1-5, 1-4, 1-3, 1-2, 1) R^{a} (e.g., R^{a} can be: hydroxyl; C₁-C₆ (e.g., C₁-C₃) alkoxy; C₃-C₇ cycloalkoxy or C₆-C₁₀ aryloxy, each of which can be optionally substituted with R^{c} and R^{d}, respectively; NR^{m}Rⁿ; or heterocyclyl including 3-8 atoms, which is optionally substituted with from 1-5 R^{c}); or

(iii) C₇-C₁₂ aralkyl, or heteroaralkyl including 6-12 atoms, each of which is optionally substituted with from 1-6 (e.g., 1-5, 1-4, 1-3, 1-2, 1) R^{c}; or

(iv) C₆-C₁₀ aryl or heteroaryl including 5-10 atoms, each of which is optionally substituted with from 1-6 (e.g., 1-5, 1-4, 1-3, 1-2, 1) R^{d}.

In certain embodiments, R^{q} can be heterocyclyl including 3-10 (e.g., 3-8, 3-6, or 5-6) atoms, which is optionally substituted with from 1-5 (1-4, 1-3, 1-2, 1) R^{c}. For example, R^{q} can be morpholinyl, piperidyl, pyrrolidinyl, or piperazinyl, each of which is optionally substituted with from 1-5 (1-4, 1-3, 1-2, 1) R^{c}.

In some embodiments, R⁶ can be C(O)NR^{m}Rⁿ. In embodiments, R^{m} and Rⁿ can each be, independently, as defined above.

In some embodiments, R⁶ can be C(O)R^{k}. In certain embodiments, R^{k} can be heterocyclyl including 3-10 (e.g., 3-8, 3-6, or 5-6) atoms, which is optionally substituted with from 1-5 (1-4, 1-3, 1-2, 1) R^{c}. For example, R^{q} can be morpholinyl, piperidyl, pyrrolidinyl, or piperazinyl, each of which is optionally substituted with from 1-5 (1-4, 1-3, 1-2, 1) R^{c}.

In some embodiments, R⁶ can be cyano.

A subset of compounds includes those in which R² has formula (C-1):

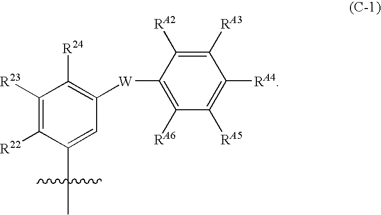

in which each of R²², R²³, and R²⁴ is, independently, hydrogen or R^{e}; and

one of R^{A2}, R^{A3}, R^{A4}, R^{A5}, and R^{A6} is R⁹, and the others are each, independently, hydrogen or R^{g}; and

R^{c}, R^{g}, and W can be as defined anywhere herein.

In certain embodiments, one of R²², R²³, and R²⁴ is hydrogen or R^{e}, and the other two are hydrogen; one of R^{A3} and R^{A4} is R⁹, the other of R^{A3} and R^{A4} is hydrogen; and each of R^{A2}, R^{A5}, and R^{A6} is, independently, hydrogen or R^{g}; and R^{e}, R^{g}, and W can be as defined anywhere herein.

Embodiments can include one or more of the following features.

W can be —O—, a bond, —OCH₂—, or —NH— (e.g., —O—, a bond, or —OCH₂—).

R^{e}, R⁹, and R^{g} can each be, independently, as defined anywhere herein.

Each of R²², R²³, and R²⁴ can be hydrogen; or each of R²², R²³, and R²⁴ can be a substituent other than hydrogen; or one or two of R²², R²³, and R²⁴ can be R^{e}, and the other(s) can be hydrogen.

One of R²², R²³, and R²⁴ can be R^{e}, and the other two can be hydrogen. For example, R²² can be R^{e}, and each of R²³ and R²⁴ can be hydrogen. In embodiments, R^{e} can be: halo (e.g., chloro); C₁-C₃ alkyl, optionally substituted with from 1-3 R^{a}; or C₁-C₃ haloalkyl (e.g., C₁-C₃ fluoroalkyl, e.g., 1-5 fluorines can be present; or C₁-C₃ perfluoroalkyl). In certain embodiments, R^{e} can be halo (e.g., chloro).

One of R^{A3} and R^{A4} can be R⁹, the other of R^{A3} and R^{A4} can be hydrogen; and each of R^{A2}, R^{A5}, and R^{A6} can be, independently, hydrogen or R^{g}.

R^{A3} can be R⁹, R^{A4} can be hydrogen, and each of R^{A2}, R^{A5}, and R^{A6} can be hydrogen; or R^{A3} can be R⁹; R^{A4} can be hydrogen; one of R^{A2}, R^{A5}, and R^{A6} (e.g., R^{A5}) can be R^{g} (e.g., halo) and the other two of R^{A2}, R^{A5}, and R^{A6} can be hydrogen.

R^{A4} can be R⁹, R^{A3} can be hydrogen, and each of R^{A2}, R^{A5}, and R^{A6} can be hydrogen. R^{A3} can be R⁹; R^{A4} can be hydrogen; one of R^{A2}, R^{A5}, and R^{A6} can be R⁹ (e.g., halo) and the other two of R^{A2}, R^{A5}, and R^{A6} can be hydrogen.

R⁹ can be —W²—S(O)ₙR¹⁰, in which n is 2, and each of W² and R¹⁰ can be as defined anywhere herein. For example, W² can be a bond. As another example, R¹⁰ can be C₁-C₁₀ alkyl, optionally substituted with from 1-2 R^{a}. In embodiments, R¹⁰ can be CH₃, CH₂CH₃, or isopropyl.

By way of example, R^{A3} can be —W²—S(O)ₙR¹⁰. n can be 2. W² can be a bond. R¹⁰ can be C₁-C₁₀ alkyl, optionally substituted with from 1-2 R^{a}. R¹⁰ can be C₁-C₃ alkyl (e.g., CH₃). R¹⁰ can be C₂-C₈ alkyl substituted with 1 R^{a} (e.g., R^{a} can be hydroxyl or C₁-C₃ alkoxy). Each of R^{A2}, R^{A4}, R^{A5}, and R^{A6} can be hydrogen. R^{A5} can be R^{g}, and each of R^{A2}, R^{A4}, and R^{A6} can be hydrogen.

R⁹ can be —W²—C(O)OR¹³. Each of W² and R¹⁰ can be as defined anywhere herein. For example, W² can be a bond or C₁-C₆ alkylene. As another example, R¹³ can be hydrogen or C₁-C₆ alkyl.

By way of example, R^{A4} can be —W²—C(O)OR¹³. W² can be a bond or C₁-C₆ alkylene (e.g., CH₂). R¹³ can be hydrogen or C₁-C₃ alkyl. Each of R^{A2}, R^{A3}, R^{A5}, and R^{A6} can be hydrogen.

Other embodiments can include one of more other features described herein and present in combination with the features delineated above.

In some embodiments, the compounds can have formula (II):

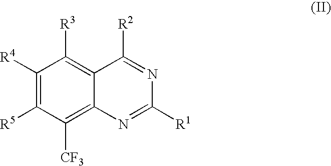

in which each of R¹, R², R³, R⁴, and R⁵ can be, independently, as defined anywhere herein (generically, subgenerically, or specifically).

In some embodiments, the compounds can have formula (III):

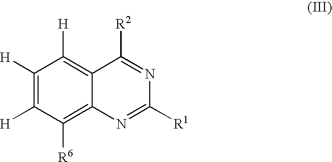

in which each of R¹, R², and R⁵ can be, independently, as defined anywhere herein (generically, subgenerically, or specifically).

In some embodiments, the compounds can have formula (IV):

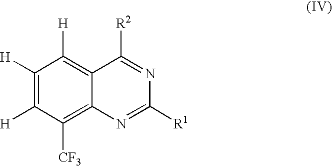

in which each of R¹, R², and R⁵ can be, independently, as defined anywhere herein (generically, subgenerically, or specifically).

In some embodiments, the compounds can have formula (V):

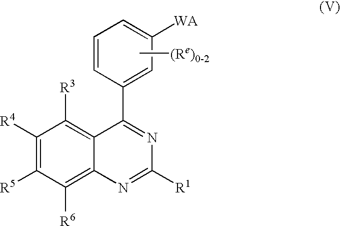

in which each of R¹, R³, R⁴, R⁵, R⁶, R^{e}, W, and A can be, independently, as defined anywhere herein (generically, subgenerically, or specifically).

In some embodiments, the compounds can have formula (VI):

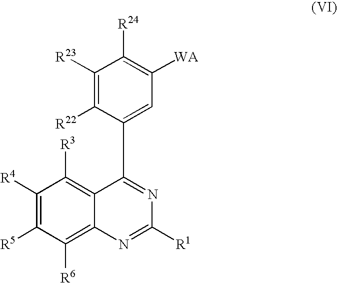

in which each of R¹, R³, R⁴, R⁵, R⁶, R²², R²³, R²⁴, W, and A can be, independently, as defined anywhere herein (generically, subgenerically, or specifically).

In some embodiments, the compounds can have formula (VII):

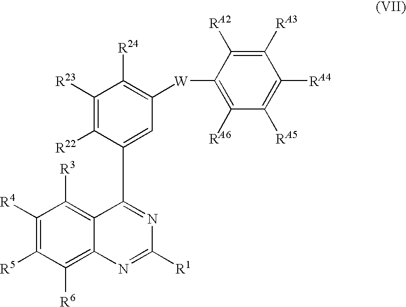

in which each of R¹, R³, R⁴, R⁵, R⁶, R²², R²³, R²⁴, R^{A2}, R^{A3}, R^{A4}, R^{A5}, R^{A6}, W, and A can be, independently, as defined anywhere herein (generically, subgenerically, or specifically).

In embodiments, the compounds of formulas (II), (III), (IV), (V), (VI), and (VII) can include any one or more of the following features below or described herein.

R¹ can be:

(i) hydrogen; or

(ii) C₁-C₆ (e.g., C₁-C₃ or C₁-C₂) alkyl or C₁-C₆ (e.g., C₁-C₃ or C₁-C₂) haloalkyl; or

(iii) C₆-C₁₀ (e.g., phenyl) or heteroaryl including 5-10 (e.g., 5-6 atoms), each of which is optionally substituted with from 1-5 R^{d}; or

(iv) C₃-C₁₀ (e.g., C₃-C₈ or C₃-C₇) cycloalkyl or heterocyclyl including 3-8 (e.g., 3-7 or 3-6) atoms, each of which is optionally substituted with from 1-3 R^{c}.

R¹ can be hydrogen.

R¹ can be:

(ii) C₁-C₆ (e.g., C₁-C₃ or C₁-C₂) alkyl or C₁-C₆ (e.g., C₁-C₃ or C₁-C₂) haloalkyl; or

(iii) C₆-C₁₀ (e.g., phenyl), which is optionally substituted with from 1-5 R^{d}; or

(iv) C₃-C₁₀ (e.g., C₃-C₈ or C₃-C₇) cycloalkyl, which is optionally substituted with from 1-3 R^{c}.

R¹ can be:

(iii) heteroaryl including 5-10 (e.g., 5-6 atoms), which is optionally substituted with from 1-5 R^{d}; or

(iv) heterocyclyl including 3-8 (e.g., 3-7 or 3-6) atoms, which is optionally substituted with from 1-3 R^{c}.

R¹ can be: H; CH₃; CF₃; or phenyl or thienyl, each of which is optionally substituted with from 1-5 R^{d}.

R² can have formula (A), (A-1), (A-2), or (C-1).

W can be —O—.

W can be a bond.

W can be —W¹(C₁₋₆ alkylene)-. In certain embodiments, W¹ can be —O—. For example, W can be —O(C₁₋₃ alkylene)- (e.g., —OCH₂—).

W can be —(C₁₋₆ alkylene)W¹—. In certain embodiments, W¹ is —NR⁹—, in which R⁹ can be hydrogen; or W¹ can be —O—. In certain embodiments, W can be —(C₁₋₃ alkylene)NH— (e.g., —CH₂NH—). In certain embodiments, W can be —(C₁₋₃ alkylene)O— (e.g., —CH₂O—).

W can be —NR⁸—, (e.g., —NH—).

In some embodiments, A can be phenyl, which is (i) substituted with 1 R⁹ and (ii) optionally substituted with from 1-4 (e.g., 1-3, 1-2, 1) R⁹, in which R⁹ can be as defined anywhere herein.

A can have formula (B-1). In embodiments, one of R^{A3} and R^{A4} is R⁹, and the other of R^{A3} and R^{A4} is hydrogen; and each of R^{A2}, R^{A5}, and R^{A6} is, independently, hydrogen or R^{g}, in which R⁹ and R^{g} can be as defined anywhere herein.

A can be heteroaryl including 5-10 atoms, which is (a) substituted with 1 R⁹; and (b) is optionally substituted with from 1-3 (e.g., 1-2, 1) R^{g}, in which R^{g} can be as defined anywhere herein.

Each of R^{e}, R⁹, and R^{g} can be, independently, as defined anywhere herein.

R⁹ can be:
—W²—S(O)ₙR¹⁰ or —W²—S(O)ₙNR¹¹R¹² (e.g., —W²—S(O)ₙR¹⁰); and/or
—W²—C(O)OR¹³.

Each of R¹⁰, R¹¹, R¹², and R¹³ can be, independently, as defined anywhere herein (e.g., as defined in conjunction with formula (C-1)).

W², n, R²², R²³, R²⁴, R^{A2}, R^{A3}, R^{A4}, R^{A5}, R^{A6} can be as defined in conjunction with formula (C-1).

Each of R³, R⁴, and R⁵ can be, independently:

(i) hydrogen; or

(ii) halo; or

(iii) C₁-C₆ (e.g., C₁-C₃) alkyl or C₁-C₆ (e.g., C₁-C₃) haloalkyl (e.g., perhaloalkyl, e.g., perfluoroalkyl), each of which is optionally substituted with from 1-3 R^{e}.

Each of R³, R⁴, and R⁵ can be hydrogen.

R⁶ can be:

(i) halo; or

(ii) C₁-C₆ alkyl or C₁-C₆ haloalkyl, each of which is optionally substituted with from 1-3 R^{a}; or

(iii) cyano; —C(O)NR^{m}Rⁿ; .—C(O)R^{k}; or —S(O)ₙR^{q}, wherein n is 1 or 2.

R⁶ can be halo (e.g., chloro) or C₁-C₆ (e.g., C₁-C₃) haloalkyl (e.g., CF₃).

A compound of the above formulae (e.g., formula VII) can have an LXRα/LXRβ binding ratio of from about 5 to about 20; from about 30 to about 39; from about 40 to about 45; or from about 54 to about 60.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species.

The compounds described herein can be synthesized according to methods described herein (or variations thereof) and/or conventional, organic chemical synthesis methods from commercially available starting materials and reagents or from starting materials and reagents that can be prepared according to conventional organic chemical synthesis methods. The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, compounds of formula (I) can be prepared according to Scheme 1.

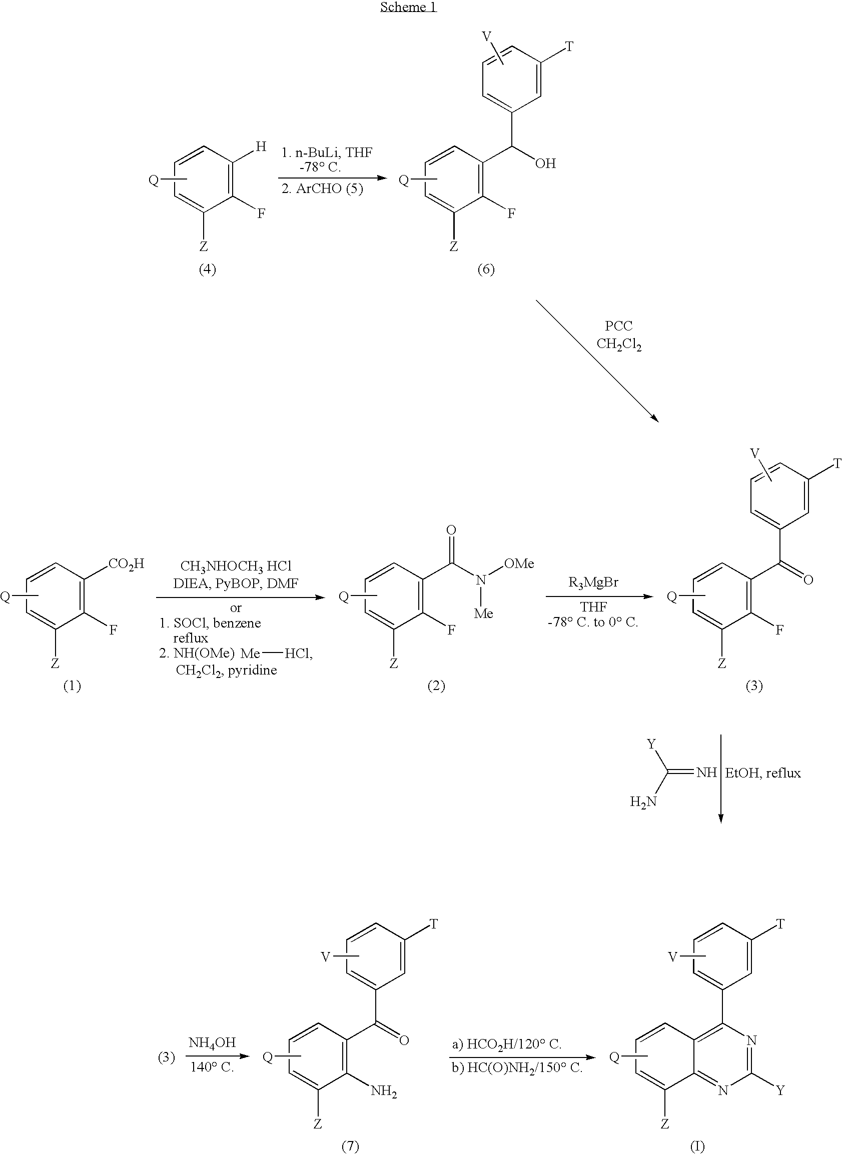

The term “Q” in Scheme 1 corresponds to R³, R⁴, and R⁵ in formula (I) or is a substituent precursor thereto. The term “Z” in Scheme 1 corresponds to R⁶ in formula (I) or is a substituent precursor thereto. The term “V” in Scheme 1 corresponds to hydrogen or R^{e} in formula (I) or is a substituent precursor thereto. The term “T” in Scheme 1 corresponds to WA in formula (I) or is a substituent precursor thereto.

According to Scheme 1, the compounds of formula (I) can be prepared be prepared by converting a benzoic acid compound, e.g., (1) to the corresponding N-methyl, N-methoxy amide (2) (sometimes referred to as a “Weinreb amide”) under conventional amidation conditions. Reaction of the amide (2) with a lithium or Grignard reagent (e.g., ArLi or ArMgBr) at low temperature can provide ketone (3). In certain embodiments, compound (4) can be lithiated alpha to the fluorine and then treated with an appropriately substituted aldehyde, e.g., (5). The resulting alcohol (6) can then be converted to the ketone (3) under conventional oxidation conditions. Conversion of (3) into the aniline of formula (7) can be accomplished using, e.g., ammonium hydroxide at elevated temperature or by using a protected amine followed by deprotection. Substituted anilines of formula (7) can undergo cyclization in the presence of formic acid with formamide at elevated temperatures to provide compounds of formula (I). In other embodiments, compounds of formula (3) can be reacted with an amidine of formula YC(NH)NH₂ at elevated temperatures, typically in ethanol at reflux or DMF in the presence of a metal carbonate base, to provide compounds of formula I. The term “Y” corresponds to R¹ in formula (I) or a substituent precursor thereto.

In some embodiments, compounds of formula (I) can be prepared according to Scheme 2.

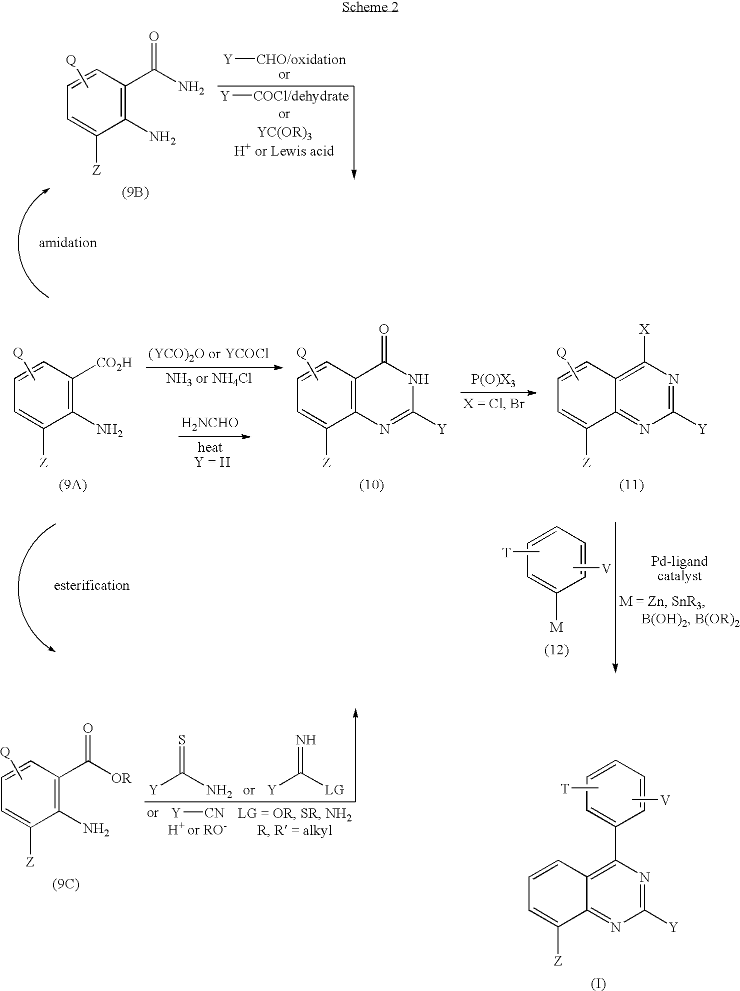

The meanings of “Q,” “Z,” “V,” “Y,” and “T” in Scheme 2 are the same as indicated above for Scheme 1.

Referring to Scheme 2, anthranilic acid derivatives, such as 9A, 9B, 9C, can be converted to into the quinazolone derivatives (10) using conventional methodologies. For example, treatment of (10) with certain phospho-halogen reagents, such as phosphorous oxychloride, phosphorous oxybromide, or other conventional similar or analogous reagents can lead to the formation of the corresponding 4-halo-quinazoline compound (11). Compound (11) can be reacted with an appropriately substituted arylboronic acid, arylzincate, or arylstannane (12) using palladium-(tetrakistriphenylphosphine) or other conventional liganded palladium catalysts to provide the compounds of formula (I).

In some embodiments, compounds of formula (I) can be prepared according to Scheme 3.

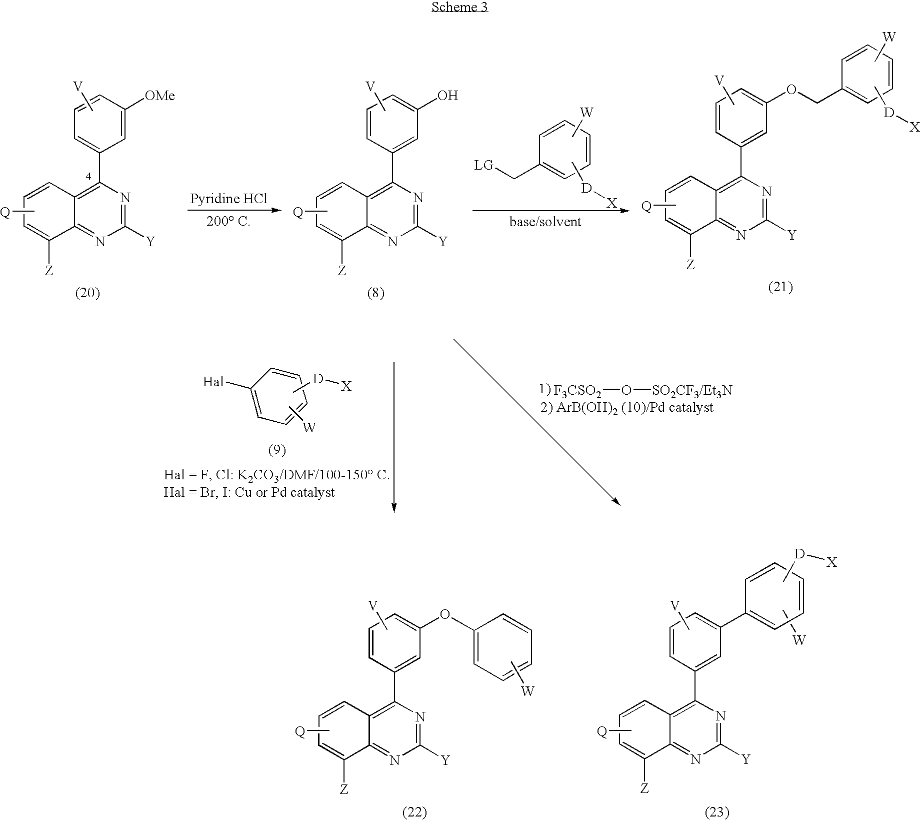

The meanings of “Q,” “Z,” “V,” and “Y” in Scheme 3 are the same as indicated above for Scheme 1. The term “W” in Scheme 3 corresponds to hydrogen or R^{g} in formula (I) or is a substituent precursor thereto. The term “D-X” in Scheme 3 corresponds to WA in formula (I) or is a substituent precursor thereto.

Referring to Scheme 3, compound (20) (which can be prepared according to the methods described in Scheme 1 or Scheme 2, i.e., compounds in which T=OMe) can be converted by conventional demethylation conditions (e.g., pyridine hydrochloride at elevated temperature or by treatment with HI in acetic acid) to provide the corresponding phenol (8). Alkylation of the OH group in (8) with an alkylating agent, e.g., RX′, using, for example, potassium, sodium or cesium carbonate as the base can provide the corresponding alkylated compound (21).

In certain embodiments, compounds that contain a carboxylic acid ester moiety can be transformed to the corresponding carboxylic acid upon treatment with, e.g., aqueous lithium, sodium or potassium hydroxide in a suitable organic solvent. In other embodiments, compounds that contain a CH2X′ moiety, in which X′ is a halogen (e.g., Br or Cl), then this halomethyl moiety can be transformed to the corresponding cyanomethy moiety, i.e., CH2CN, upon treatment with, e.g., sodium cyanide in a suitable organic solvent.

Referring back to Scheme 3, compound (8) (which can be prepared according to the methods described in Scheme 1 or Scheme 2, i.e., compounds in which T=OH) can be treated with a halogenated aromatic ring, e.g., compound (9), to provide biarylether (22). In certain embodiments, the halogen (Hal in Scheme 3) can be a fluorine or chlorine atom, and formation of the biarylether of formula (I) can be accomplished using a base such as potassium carbonate, typically in a polar solvent such as dimethylformamide or dimethylsulfoxide, at elevated temperatures, typically 100° C. to 150° C. for several hours. In other embodiments, the halogen (Hal in Scheme 3) can be a bromine or iodine atom, and the formation of biarylether (22) can be accomplished using a metal catalyst such as a copper salt or a palladium salt in the presence of a base and a solvent such as dioxane at elevated temperatures.

Referring again to Scheme 3, compound (8) can be converted to the corresponding triflate (structure not shown in Scheme 3) using, e.g., triflic anhydride and a tertiary amine such as triethylamine. The triflate (or bromide) can be coupled to aryl boronic acid (10) under catalysis with a palladium catalyst (this transformation is sometimes referred to as a “Suzuki reaction”).

In some embodiments, compounds of formula (I) can be prepared according to Scheme 4.

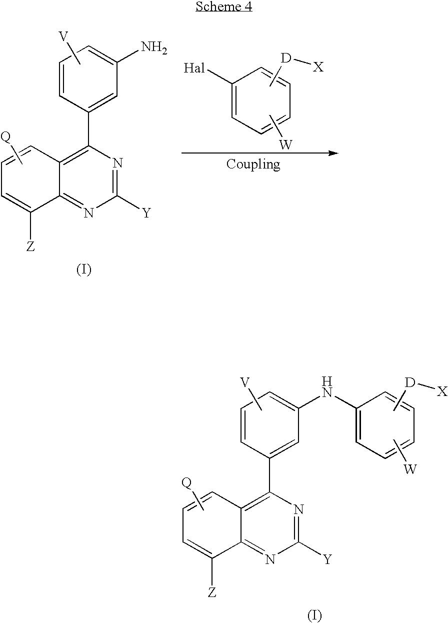

The meanings of “Q,” “Z,” “V,” “Y,” “W,” and “D-X” in Scheme 4 are the same as indicated above for Schemes 1 and 3.

Referring to Scheme 4, aryl amine (24) can be coupled to an optionally substituted aryl halide (or aryltriflate or arylboronic acid) using, e.g., (±)2,2′-bis(diphenylphosphino)-1,1′-binaphthalene (BINAP) as a palladium ligand, Cs₂CO₃ as a base, and Pd(OAc)₂ as the catalyst for the coupling, typically heating in toluene at reflux for 2 to 6 h. Alternatively, the coupling can be performed using stoichiometric Cu(OAc)₂ with triethylamine in dichloromethane, at ambient temperature open to air, for typically 18-24 h to provide biarylamine (25).

In certain embodiments, amine or phenol (collectively shown as (26) in Scheme 5) can be arylated with a boronic acid in the presence of a base, e.g., 2,6-lutidine, an additive such as myristic acid, and Cu(OAc)₂ in an inert solvent such as toluene at room temperature or elevated temperatures to provide biarylamine (25).

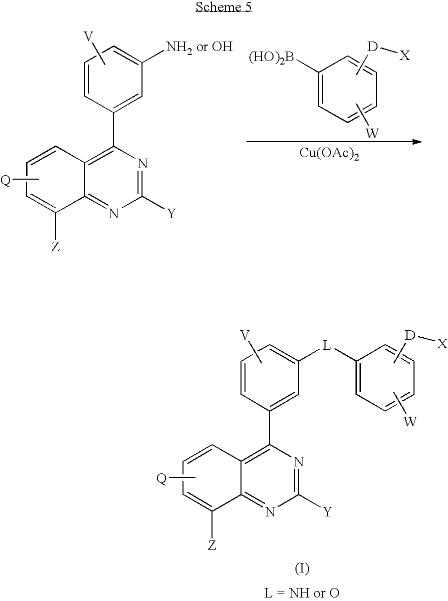

The meanings of “Q,” “Z,” “V,” “Y,” “W,” and “D-X” in Scheme 5 are the same as indicated above for Schemes 1 and 3.

In some embodiments, the compounds of formula (I) can be prepared using halogenated sulfone or sulfonamide intermediates.

Halogenated Arylsulfones

Halogenated arylsulfones can be prepared by conventional methods.

In certain embodiments, halogenated arylsulfones can be prepared via partial reduction of halogenated arylsulfonyl chlorides (which can be obtained from commercial sources or by conventional synthetic methods) using sodium sulfite and sodium bicarbonate in water, typically at 95-100° C. for 0.5 to 1 h to provide the sodium arylsulfinate. Typically, the reduction reaction mixture is cooled, treated with an alkylating agent such as an alkylating agent (e.g., R-LG in which LG is a leaving group such as a bromide, iodine, or tosylate). Useful alkylating agents can include, without limitation, primary alkyl halides, e.g., ethyl iodide and 3-bromopropan-1-ol. Typically, a phase transfer catalyst in the alkylation step, e.g., tetrabutylammonium bromide, and the two-phase mixture is heated at 40-100° C. for several hours to provide the halogenated arylsulfones (see Scheme 6).

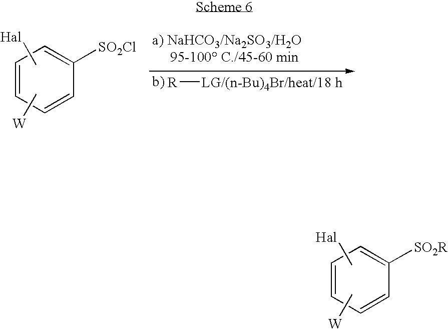

The term “W” in Scheme 6 corresponds to hydrogen or R^{g} in formula (I) or is a substituent precursor thereto.

In other embodiments, halogenated thiophenols can be alkylated with an alkylating agent in the presence of a base, typically potassium carbonate, in an appropriate solvent such as acetone. The reaction is typically heated at 40 to 65° C. for 1-4 h, cooled, and treated with aqueous sodium bicarbonate and Oxone®. Typically after 18-48 h, the desired halogenated arylsulfones can be isolated (see Scheme 7).

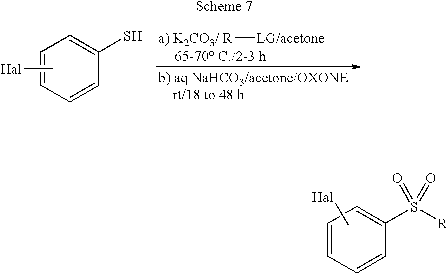

In still other embodiments, aryl bromides and iodides can be converted to halogenated arylsulfones, e.g., methylsulfones, using a copper-catalyzed coupling reaction employing sodium methylsulfinate (see Scheme 8).

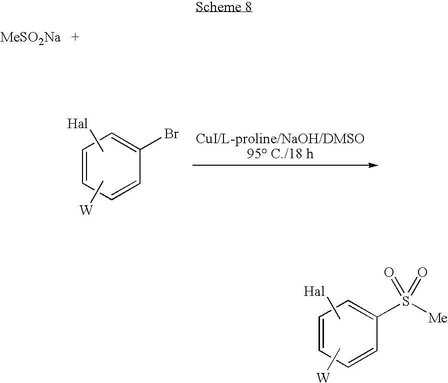

Halogenated Arylsulfonamides

Halogenated arylsulfonamides can be prepared, e.g., by reaction of halogenated arylsulfonyl chlorides with amines (see Scheme 9).

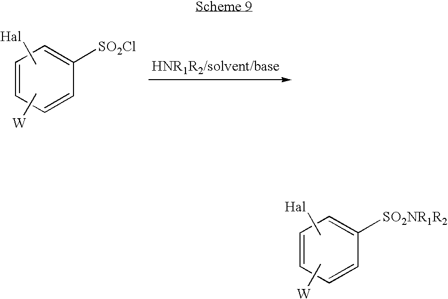

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In general, the compounds described herein can be used for treating (e.g., controlling, ameliorating, preventing, delaying the onset of, or reducing the risk of developing) one or more diseases, disorders, conditions or symptoms mediated by LXRs (e.g., cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A disorder or physiological condition that is mediated by LXR refers to a disorder or condition wherein LXR can trigger the onset of the condition, or where inhibition of a particular LXR can affect signaling in such a way so as to treat, control, ameliorate, prevent, delay the onset of, or reduce the risk of developing the disorder or condition. Examples of such disorders include, but are not limited to cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

While not wishing to be bound by theory, it is believed that LXR modulators that activate cholesterol efflux (e.g., upregulate ABCA1), but do not substantially increase SREBP-1c expression and triglyceride synthesis in liver, can both reduce atherosclerotic risk and minimize the likelihood of concomitantly increasing serum and hepatic triglyceride levels. Candidate compounds having differential activity for regulating ABCA1 (ABCG1) vs. SREBP-1c can be can be evaluated using conventional pharmacological test procedures, which measure the affinity of a candidate compound to bind to LXR and to upregulate the gene ABCA1.

In some embodiments, LXR ligands can be identified initially in cell-free LXR beta and LXR alpha competition binding assays. LXR ligands can be further characterized by gene expression profiling for tissue selective gene regulation.

In some embodiments, the compounds described herein have agonist activity for ABCA1 transactivation but do not substantially affect (e.g., inhibit) SREBP-1c gene expression in differentiated THP-1 macrophages. Gene expression analysis in an antagonist mode can be used to further delineate differential regulation of ABCA1 and SREBP-1c gene expression. In certain embodiments, the compounds described herein preferentially antagonize SREBP-1c activation (a marker for genes involved in cholesterol and fatty acid homeostasis) but do not substantially affect (e.g., have relatively minimal or additive effects) on ABCA1 gene expression or genes known to enhance HDL biogenesis (based on a competition assay with known potent synthetic LXR agonists). Cell type or tissue specificity may be further evaluated in additional cell lines, intestinal, CaCo2 or liver, HepG2 and Huh-7 cells where ABCA1 activity is believed to influence net cholesterol absorption and reverse cholesterol transport. The test procedures performed, and results obtained therefrom are described in the Examples section.

In some embodiments, the compounds described herein have agonist activity for ABCA1 and antagonist activity for SREBP-1c (e.g., as determined by gene specific modulation in cell based assays). In certain embodiments, the compounds described herein (in the agonist mode) have at least about 20% efficacy for ABCA1 activation by LXR and do not substantially agonize SREBP-1c (at most about 25% efficacy relative to a reference compound N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838)). In certain embodiments, the compounds described herein (in the antagonist mode) do not substantially antagonize ABCA1 gene expression. While not wishing to be bound by theory, it is believed that there may be an additive effect on ABCA1 gene expression relative to the reference compound at their EC₅₀ concentration. In certain embodiments, the compounds described herein (in the antagonist mode) inhibited agonist-mediated SREBP-1c gene expression in a dose dependent fashion.

In some embodiments, to study the effect of the compounds of formula (I) on skin aging, for example, in a clinical trial, cells can be isolated and RNA prepared and analyzed for the levels of expression of TIMP1, ABCA12, decorin, TNFα, MMP1, MMP3, and/or IL-8. The levels of gene expression (i.e., a gene expression pattern) can be quantified, for example, by Northern blot analysis or RT-PCR, by measuring the amount of protein produced, or by measuring the levels of activity of TIMP1, ABCA12, decorin, TNFα, MMP1, MMP3, and/or IL-8, all by methods known to those of ordinary skill in the art. In this way, the gene expression pattern can serve as a marker, indicative of the physiological response of the cells to the compounds of formula (I). Accordingly, this response state may be determined before, and at various points during, treatment of the individual with the compounds of formula (I).

In one embodiment, expression levels of cytokines and metalloproteases described herein can be used to facilitate design and/or identification of compounds that treat skin aging through an LXR-based mechanism. Accordingly, the invention provides methods (also referred to herein as “screening assays”) for identifying modulators, i.e., LXR modulators, that have a stimulatory or inhibitory effect on, for example, TIMP1, ABCA12, decorin, TNFα, MMP1, MMP3, and/or IL-8 expression.

An exemplary screening assay is a cell-based assay in which a cell that expresses LXR is contacted with a test compound, and the ability of the test compound to modulate TIMP1, ABCA12, decorin, TNFα, MMP1, MMP3, and/or IL-8 expression through an LXR-based mechanism. Determining the ability of the test compound to modulate TIMP1, ABCA12, decorin, TNFα MMP1, MMP3, and/or IL-8 expression can be accomplished by monitoring, for example, DNA, mRNA, or protein levels, or by measuring the levels of activity of TIMP1, ABCA12, decorin, TNFα, MMP1, MMP3, and/or IL-8, all by methods known to those of ordinary skill in the art. The cell, for example, can be of mammalian origin, e.g., human.

In some embodiments, to study the effect of the compounds of formula (I) on osteoarthritis, for example, in a clinical trial, cells can be isolated and RNA prepared and analyzed for the levels of expression of ApoD and other genes implicated in osteoarthritis (for example, TNFα). The levels of gene expression (i.e., a gene expression pattern) can be quantified by Northern blot analysis or RT-PCR, by measuring the amount of protein produced, or by measuring the levels of activity of ApoD or other genes, all by methods known to those of ordinary skill in the art. In this way, the gene expression pattern can serve as a marker, indicative of the physiological response of the cells to the LXR modulator. Accordingly, this response state may be determined before, and at various points during, treatment of the individual with the LXR modulator.

An exemplary screening assay is a cell-based assay in which a cell that expresses LXR is contacted with a test compound, and the ability of the test compound to modulate ApoD expression and/or aggrecanase activity and/or cytokine elaboration through an LXR-based mechanism. Determining the ability of the test compound to modulate ApoD expression and/or aggrecanase activity and/or cytokine elaboration can be accomplished by monitoring, for example, DNA, mRNA, or protein levels, or by measuring the levels of activity of ApoD, aggrecanase, and/or TNFα, all by methods known to those of ordinary skill in the art. The cell, for example, can be of mammalian origin, e.g., human.

In some embodiments, the compounds described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of one or more compounds of formula (I) (including any subgenera or specific compounds thereof)). Alternatively, these agents may be part of a single dosage form, mixed together with the compounds of this invention in a single composition. In still another embodiment, these agents can be given as a separate dose that is administered at about the same time that one or more compounds of formula (I) (including any subgenera or specific compounds thereof) are administered (e.g., simultaneously with the administration of one or more compounds of formula (I) (including any subgenera or specific compounds thereof)). When the compositions of this invention include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/Kg to about 1000 mg/Kg, (e.g., from about 0.01 to about 100 mg/kg, from about 0.1 to about 100 mg/Kg, from about 1 to about 100 mg/Kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, New York, 537 (1970). In certain embodiments, the compositions are administered by oral administration or administration by injection. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Chapter 67 of Remington's Pharmaceutical Sciences, 15th Edition (1975) published by Mack Publishing Company.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

The invention will be further described in the following examples. It should be understood that these examples are for illustrative purposes only and are not to be construed as limiting this invention in any manner.

### EXAMPLES

The following describes the preparation of representative compounds of this invention. Compounds described as homogeneous are determined to be of 90% or greater purity (exclusive of enantiomers) by analytical reverse phase chromatographic analysis with 254 nM UV detection. Melting points are reported as uncorrected in degrees centigrade. Mass spectral data is reported as the mass-to-charge ratio, m/z; and for high resolution mass spectral data, the calculated and experimentally found masses, [M+H]⁺, for the neutral formulae M are reported. All reactions are stirred and run under a nitrogen atmosphere unless otherwise noted.

### Example 1

### 4-(3-methoxyphenyl)-8-(trifluoromethyl)quinazoline

A stirred mixture of (2-amino-3-(trifluoromethyl)phenyl)(3-methoxyphenyl)methanone (2.95 g, 10.0 mmol), formic acid (5 mL), and formamide (20 mL) was heated for 4 h at 150° C., under a nitrogen atmosphere. After 1 h, reaction was cooled, poured into ice water (150 mL), and extracted with dichloromethane (2×50 mL). The combined extracts were dried (MgSO₄) and concentrated in vacuo. Chromatography on silica gel (15/85 to 30/70 E/H gradient) gave the title compound as a white solid (1.04 g, R_{f}˜0.5 in 35/65 E/H). MS (ES) m/z 304.6; HRMS: calcd for C₁₆H₁₁F₃N₂O+H⁺, 305.08962; found (ESI, [M+H]⁺), 305.0896.

### Example 2

### 3-(8-(Trifluoromethyl)quinazolin-4-yl)phenol

A stirred mixture of 4-(3-methoxyphenyl)-8-(trifluoromethyl)quinazoline (915 mg, 3.00 mmol) and pyridine hydrochloride (6.5 g) was heated at 200-205° C., under a nitrogen atmosphere. After 2.5 h, reaction was poured into water (50 mL), treated with aqueous saturated NaHCO₃ (100 mL) and extracted with dichloromethane (100 mL). The combined extracts were dried (MgSO₄) and concentrated in vacuo. Chromatography on silica gel (30/70 to 60/40 E/H gradient) gave the title compound as a white solid (810 mg, R_{f}˜0.3 in 50/50 E/H). MS (ES) m/z 290.9.

### Example 3

### 3-[2-methyl-8-(trifluoromethyl)quinazolin-4-yl]phenol

Prepared according to a procedure similar to that described in Example 2 except using 4-(3-methoxyphenyl)-2-methyl-8-(trifluoromethyl)quinazoline. MS (ES) m/z 305.0.

### Example 4

### 4-{3-[3-(Methyl sulfonyl)phenoxy]phenyl}-8-(trifluoromethyl)quinazoline

A stirred mixture of 3-(8-(trifluoromethyl)quinazolin-4-yl)phenol (116 mg, 0.40 mmol), 3-(fluorophenyl)-methylsulfone (104 mg, 0.60 mmol), and potassium carbonate (110 mg, 0.80 mmol) in DMF (2 mL) was heated at 150-155° C., under a nitrogen atmosphere. After 19 h, the reaction was poured into water (15 mL), brine (3 mL), and extracted with ethyl acetate (3×10 mL). The combined extracts were dried (MgSO₄) and concentrated in vacuo. Chromatography on silica gel (30/70 to 60/40 E/H gradient) gave the title compound as a white solid (120 mg, R_{f}˜0.25 in 50/50 E/H). MS (ES) m/z 444.9

### Example 5

### 4-{3-[3-(ethylsulfonyl)phenoxy]phenyl}-8-(trifluoromethyl)quinazoline

Prepared according to a procedure similar to that described in Example 4 except using 3-(fluorophenyl)-ethylsulfone. MS (ES) m/z 458.9; HRMS: calcd for C₂₃H₁₇F₃N₂O₃S+H⁺, 459.09847; found (ESI, [M+H]⁺), 459.0963.

### Example 6

### 4-{3-[3-(isopropylsulfonyl)phenoxy]phenyl}-8-(trifluoromethyl)quinazoline

Prepared according to a procedure similar to that described in Example 4 except using 3-(fluorophenyl)-isopropylsulfone. MS (ES) m/z 472.8; HRMS: calcd for C₂₄H₁₉F₃N₂O₃S+H⁺, 473.11412; found (ESI, [M+H]⁺), 473.116.

### Example 7

### 4-(3-{3-[(3-methoxypropyl)sulfonyl]phenoxy}phenyl)-8-(trifluoromethyl)quinazoline

Prepared according to a procedure similar to that described in Example 4 except using 3-(fluorophenyl)-3-methoxypropylsulfone. MS (ES) m/z 502.8; HRMS: calcd for C₂₅H₂₁F₃N₂O₄S+H⁺, 503.12469; found (ESI, [M+H]⁺), 503.1246.

### Example 8

### 4-{3-[3-chloro-5-(propylsulfonyl)phenoxy]phenyl}-8-(trifluoromethyl)quinazoline

Prepared according to a procedure similar to that described in Example 4 except using 3-(fluorophenyl)-propylsulfone. MS (ES) m/z 506.8; HRMS: calcd for C₂₄H₁₈ClF₃N₂O₃S+H⁺, 507.07515; found (ESI, [M+H]⁺), 507.0744.

### Example 9

### 3-[(3-chloro-5-{3-[8-(trifluoromethyl)quinazolin-4-yl]phenoxy}phenyl)sulfonyl]propan-1-ol

Prepared according to a procedure similar to that described in Example 4 except using 3-(fluorophenyl)-3-hydroxypropylsulfone. MS (ES) m/z 522.8; HRMS: calcd for C₂₄H₁₈ClF₃N₂O₄S+H⁺, 523.07006; found (ESI, [M+H]⁺), 523.0712.

### Example 10

### 4-{3-[4-(methylsulfonyl)phenoxy]phenyl}-8-(trifluoromethyl)quinazoline

Prepared according to a procedure similar to that described in Example 4 except using 4-(fluorophenyl)-methylsulfone. MS (ES) m/z 444.8; HRMS: calcd for C₂₂H₁₅F₃N₂O₃S+H⁺, 445.08282; found (ESI, [M+H]⁺), 445.0834.

### Example 11

### 4-{3-[4-(ethylsulfonyl)phenoxy]phenyl}-8-(trifluoromethyl)quinazoline

Prepared according to a procedure similar to that described in Example 4 except using 4-(fluorophenyl)-ethylsulfone. MS (ES) m/z 458.9; HRMS: calcd for C₂₃H₁₇F₃N₂O₃S+H⁺, 459.09847; found (ESI, [M+H]⁺), 459.0983.

### Example 12

### 4-{3-[4-(propylsulfonyl)phenoxy]phenyl}-8-(trifluoromethyl)quinazoline

Prepared according to a procedure similar to that described in Example 4 except using 4-(fluorophenyl)-propylsulfone. MS (ES) m/z 472.8; HRMS: calcd for C₂₄H₁₉F₃N₂O₃S+H⁺, 473.11412; found (ESI, [M+H]⁺), 473.114.

### Example 13

### 4-{3-[4-(isopropylsulfonyl)phenoxy]phenyl}-8-(trifluoromethyl)quinazoline

Prepared according to a procedure similar to that described in Example 4 except using 4-(fluorophenyl)-isopropylsulfone. MS (ES) m/z 472.9; HRMS: calcd for C₂₄H₁₉F₃N₂O₃S+H⁺, 473.11412; found (ESI, [M+H]⁺), 473.1132.

### Example 14

### 4-{3-[2-(methylsulfonyl)phenoxy]phenyl}-8-(trifluoromethyl)quinazoline

Prepared according to a procedure similar to that described in Example 4 except using 2-(fluorophenyl)-methylsulfone. MS (ES) m/z 444.9; HRMS: calcd for C₂₂H₁₅F₃N₂O₃S+H⁺, 445.08282; found (ESI, [M+H]⁺), 445.0817.

### Example 15

### 4-{3-[3-(propylsulfonyl)phenoxy]phenyl}-8-(trifluoromethyl)quinazoline

Prepared according to a procedure similar to that described in Example 4 except using 3-(fluorophenyl)-propylsulfone. MS (ES) m/z 472.7.

### Example 16

### 4-{3-[3-(isobutylsulfonyl)phenoxy]phenyl}-8-(trifluoromethyl)quinazoline

Prepared according to a procedure similar to that described in Example 4 except using 3-(fluorophenyl)-isobutylsulfone. MS (ES) m/z 486.8.

### Example 17

### 4-(3-{3-[(3-methylbutyl)sulfonyl]phenoxy}phenyl)-8-(trifluoromethyl)quinazoline

Prepared according to a procedure similar to that described in Example 4 except using 3-(fluorophenyl)-(3-methylbutyl)sulfone. MS (ES) m/z 500.8.

### Example 18

### 3-[(3-{3-[8-(trifluoromethyl)quinazolin-4-yl]phenoxy}phenyl)sulfonyl]propan-1-ol

Prepared as in Example 4 except using 3-(fluorophenyl)-(3-hydroxylpropyl)sulfone. MS (ES) m/z 488.7.

### Example 19

### 3-[(4-{3-[8-(trifluoromethyl)quinazolin-4-yl]phenoxy}phenyl)sulfonyl]propan-1-ol

Prepared according to a procedure similar to that described in Example 4 except using 4-(fluorophenyl)-(3-hydroxylpropyl)sulfone. MS (ES) m/z 488.7.

### Example 20

### 4-{3-[3-fluoro-5-(methylsulfonyl)phenoxy]phenyl}-8-(trifluoromethyl)quinazoline

Prepared according to a procedure similar to that described in Example 4 except using 3,5-(difluorophenyl)-methylsulfone. MS (ES) m/z 463.1; HRMS: calcd for C₂₂H₁₄F₄N₂O₃S+H⁺, 463.07340; found (ESI, [M+H]⁺), 463.0725.

### Example 21

### 2-methyl-4-{3-[3-(methylsulfonyl)phenoxy]phenyl}-8-(trifluoromethyl)quinazoline

Prepared according to a procedure similar to that described in Example 4 except using 3-[2-methyl-8-(trifluoromethyl)quinazolin-4-yl]phenol. MS (ES) m/z 459.0.

### Example 22

### 3-(3-(methylsulfonyl)phenoxy)benzonitrile

A stirred mixture of 3-(fluorophenyl)-methylsulfone (3.48 g, 20.0 mmol), 3-hydroxybenxonitrile (3.60 g, 30.0 mmol), and potassium carbonate (4.20 g, 30.0 mmol) in NMP (25 mL) was heated at 160° C. under a nitrogen atmosphere for 42 h. The reaction was diluted with water (150 mL) and extracted with dichloromethane (2×60 mL). The extracts were dried (MgSO₄) and concentrated in vacuo to an oil. Chromatography on silica gel using a 20/80 to 40/60 E/H gradient followed by trituration with 20/80 E/H gave the title compound as white flakes (2.20 g, R_{f}˜0.15 in 35/65 E/H).

### Example 23

### (2-amino-3-chlorophenyl)(3-(3-(methylsulfonyl)phenoxy)phenyl)methanone

A stirred mixture of 1.0 M borontrichloride in p-xylenes (3.9 mL, 3.9 mmol) in 1,2-dichloroethane (10 mL) was cooled in an ice bath and treated with 2-chloroaniline (765 mg, 6.0 mmol) in 1,2-dichloroethane (5.0 mL). After 15 min, the cold bath was removed and a solution of 3-(3-(methylsulfonyl)phenoxy)benzonitrile (820 mg, 3.0 mmol) in 1,2-dichloroethane (5.0 mL) was added followed by aluminum trichloride (0.53 g, 3.90 mmol). The reaction was heated at 85° C. for 17 h, cooled, treated with ice (10 g) and 2M hydrochloric acid (5 mL), and heated at reflux for 1 h. The reaction was cooled and extracted with dichloromethane (2×15 mL). The extracts were dried (MgSO₄) and concentrated in vacuo. Chromatography (30/70 to 50/50 E/H) gave the title compound as an oil (235 mg, R_{f}˜0.35 in 50/50 E/H).

### Example 24

### 8-chloro-4-{3-[3-(methylsulfonyl)phenoxy]phenyl}quinazoline

Prepared according to a procedure similar to that described in Example 1. MS (ES) m/z 410.9; HRMS: calcd for C₂₁H₁₅ClN₂O₃S+H⁺, 411.05647; found (ESI, [M+H]⁺), 411.0557.

### Example 25

### 4-(3-{[3-(methylsulfonyl)benzyl]oxy}phenyl)-8-(trifluoromethyl)quinazoline

A stirred mixture of 3-(8-(trifluoromethyl)quinazolin-4-yl)phenol (145 mg, 0.30 mmol), 3-(bromomethylphenyl)-methylsulfone (137 mg, 0.55 mmol), and 60% sodium hydride in mineral oil (24 mg, 0.60 mmol) in DMF (2 mL) was stirred at 20° C. for 3 h, under a nitrogen atmosphere. The reaction was treated with water (20 mL), NH₄Cl (100 mg), and extracted with ethyl acetate (2×10 mL). The combined extracts were dried (MgSO₄) and concentrated in vacuo. Chromatography on silica gel (20/80 to 50/50 E/H gradient) gave the title compound as a white solid (155 mg, R_{f}˜0.40 in 50/50 E/H).

MS (ES) m/z 459.1

### Example 26

### 4-(3-Bromo-phenyl)-8-trifluoromethyl-quinazoline

A stirred mixture of (2-amino-3-(trifluoromethyl)phenyl)(3-bromophenyl)-methanone (1.0 g, 2.9 mmol), triethylorthoformate (5 mL), and H₂SO₄ (cat) was heated for 12 h at 130° C., under a nitrogen atmosphere with removal of ethanol. After 12 h a slight vacuum was placed on the reaction and it was brought to a thick liquid. The reaction was cooled and an excess of ammonium acetate in 4 mL of ethanol was added and the reaction was heated for 5 h at 100° C. The reaction was cooled, poured into ice water (150 mL), and extracted with ethyl acetate (2×50 mL). The combined extracts were dried (MgSO₄) and concentrated in vacuo. Chromatography on silica gel (15/85 to 30/70 E/H gradient) gave the title compound as a white solid (0.85 g). MS (ES) m/z 353.1.

### Example 27

### 4-(3′-Methanesulfonyl-biphenyl-3-yl)-8-trifluoromethyl-quinazoline

4-(3-Bromo-phenyl)-8-trifluoromethyl-quinazoline (0.10 g, 0.27 mmol) in toluene (3 mL) and ethanol (0.5 mL) was treated with 3-(methanesulfonyl)benzeneboronic acid (0.30 mmol), 2 M aqueous Na₂CO₃ (0.25 mL, 0.50 mmol), and Pd(PPh₃)₄ (9 mg, 0.0075 mmol). The reaction was heated at 90° C. for 8 h. The solvent was removed and the residue chromatographed using 10:90 ethyl acetate:hexane to obtain 0.051 g of the title compound. MS (ES) m/z 428.1.

### Example 28

### 4-(4′-Methanesulfonyl-biphenyl-3-yl)-8-trifluoromethyl-quinazoline

Prepared according to a procedure similar to that described in Example 27 except using 4-(methanesulfonyl)benzeneboronic acid MS (ES) m/z 428.1.

### Example 29

### 4-(3-bromophenyl)-2-phenyl-8-(trifluoromethyl)quinazoline

To a stirred solution of benzimidamide hydrochloride (1.2 g, 7.6 mmol), in ethanol (5 mL) was added 1.7 mL of 5 mM sodium ethoxide solution. After stirring for 20 min, a solution of (3-bromophenyl)(2-fluoro-3-(trifluoromethyl)phenyl)methanone (1.7 g, 4.9 mmol) in ethanol was added and the reaction mixture brought to reflux for 4 h. The reaction was cooled and diluted with saturated aqueous ammonium chloride and extracted with ethyl acetate (2×50 mL). The combined extracts were dried (MgSO₄) and concentrated in vacuo. Chromatography on silica gel (15/85 to 30/70 E/H gradient) gave the title compound as a white solid (1.2 g). (60%) MS (ES) m/z 429.2.

### Example 30

### 4-(3-bromophenyl)-2-(thiophen-2-yl)-8-(trifluoromethyl)quinazoline

Prepared according to a procedure similar to that described in Example 29 except using thiophene-2-carboximidamide hydrochloride. MS (ES) m/z 434.6.

### Example 31

### 4-(3-Bromo-phenyl)-2-methyl-8-trifluoromethyl-quinazoline

To a stirred solution of (2-amino-3-(trifluoromethyl)phenyl)(3-bromo-phenyl)methanone (1.00 g, 2.9 mmol), in acetonitrile (15 mL) at 0° C. was bubbled HCl (gas) until saturation. The vessel was sealed and heated for 14 h at 50° C. After 14 h the reaction was cooled and concentrated in vacuo to give an oily residue, which was taken up in NaHCO₃ solution (sat) and extracted with ethyl acetate (2×50 mL). The combined extracts were dried (MgSO₄) and concentrated in vacuo. Chromatography on silica gel (15/85 to 30/70 E/H gradient) gave the title compound as a white solid (0.90 g).

### Example 32

### 4-(3-bromophenyl)-2,8-bis(trifluoromethyl)quinazoline

Prepared according to a procedure similar to that described in Example 31 except using 2,2,2-trifluoroacetonitrile. MS (ES) m/z 420.7.

### Example 33

### 4-(3-bromophenyl)-2-ethyl-8-(trifluoromethyl)quinazoline

Prepared according to a procedure similar to that described in Example 31 except using propiononitrile. MS (ES) m/z 380.5.

### Example 34

### 4-(3-bromophenyl)-2-propyl-8-(trifluoromethyl)quinazoline

Prepared according to a procedure similar to that described in Example 31 except using butyronitrile. MS (ES) m/z 394.6.

### Example 35

### 4-(3-bromophenyl)-2-isopropyl-8-(trifluoromethyl)quinazoline

Prepared according to a procedure similar to that described in Example 31 except using isobutyronitrile. MS (ES) m/z 394.6.

### Example 36

### 4-(3-methoxyphenyl)-2-methyl-8-(trifluoromethyl)quinazoline

Prepared according to a procedure similar to that described in Example 31 except using (2-amino-3-(trifluoromethyl)phenyl)(3-methoxyphenyl)methanone. MS (ES) m/z 318.8.

### Example 37

### 4-(3′-Methanesulfonyl-biphenyl-3-yl)-2-methyl-8-trifluoromethyl-quinazoline

4-(3-Bromo-phenyl)-2-methyl-8-trifluoromethyl-quinazoline (0.20 g, 0.54 mmol) in toluene (3 mL) and ethanol (0.5 mL) was treated with 3-(methanesulfonyl)-benzeneboronic acid (0.30 mmol), 2 M aqueous Na₂CO₃ (0.25 mL, 0.50 mmol), and Pd(PPh₃)₄ (9 mg, 0.0075 mmol). The reaction was heated at 90° C. for 8 h. The solvent was removed and the residue chromatographed using 10:90 ethyl acetate:hexane to obtain 0.051 g of the title compound. MS (ES) m/z 442.5.

### Example 38

### 4-(4′-Methanesulfonyl-biphenyl-3-0)-2-methyl-8-trifluoromethyl-quinazoline

Prepared according to a procedure similar to that described in Example 37 except using 4-(methanesulfonyl)benzeneboronic acid. MS (ES) m/z 442.5.

Compounds below were prepared according to a procedure similar to that described in Example 37, using the appropriate aryl bromide or triflate and boronic acids, varying reaction times up to 12 h.

### Example 39

### 4-[3′-(methylsulfonyl)biphenyl-3-yl]-2,8-bis(trifluoromethyl)quinazoline

Prepared according to a procedure similar to that described in Example 37 except using 4-(3-bromophenyl)-2,8-bis(trifluoromethyl)-quinazoline and 3-(methanesulfonyl)benzeneboronic acid. MS (ES) m/z 496.4.

### Example 39a

### 4-[4-chloro-3′-(methylsulfonyl)biphenyl-3-yl]-8-(trifluoromethyl)quinazoline

Prepared according to a procedure similar to that described in Example 37 except using 4-chloro-3-(8-(trifluoromethyl)quinazolin-4-yl)phenyl trifluoromethanesulfonate and 3-(methanesulfonyl)benzeneboronic acid. MS (ES) m/z 462.8.

### Example 40

### 2-ethyl-4-[3′-(methylsulfonyl)biphenyl-3-yl]-8-(trifluoromethyl)quinazoline

### Step 1: 4-(3-bromophenyl)-2-ethyl-8-(trifluoromethyl)quinazoline

Prepared as in Example 31 except using propiononitrile. MS (ES) m/z 380.5; HRMS: calcd for C₁₇H₁₂BrF₃N₂+H⁺, 381.02087; found (ESI, [M+H]⁺ Obs'd), 381.0209.

### Step 2: 2-ethyl-4-[3′-(methylsulfonyl)biphenyl-3-yl]-8-(trifluoromethyl)quinazoline

Prepared according to a procedure similar to that described in Example 37 except using 4-(3-bromophenyl)-2-ethyl-8-(trifluoromethyl)-quinazoline and 3-(methanesulfonyl)benzeneboronic acid. MS (ES) m/z 456.7.

### Example 41

### 4-[3′-(methylsulfonyl)biphenyl-3-yl]-2-propyl-8-(trifluoromethyl)quinazoline

### Step 1: 4-(3-bromophenyl)-2-propyl-8-(trifluoromethyl)quinazoline

Prepared as in Example 31 except using butyronitrile. MS (ES) m/z 394.6; HRMS: calcd for C₁₈H₁₄BrF₃N₂+H⁺, 395.03652; found (ESI, [M+H]⁺Calc'd), 395.0365.

### Step 2: 4-[3′-(methylsulfonyl)biphenyl-3-yl]-2-propyl-8-(trifluoromethyl)quinazoline

Prepared according to a procedure similar to that described in Example 37 except using 4-(3-bromophenyl)-2-propyl-8-(trifluoromethyl)-quinazoline and 3-(methanesulfonyl)benzeneboronic acid. MS (ES) m/z 470.7.

### Example 42

### 2-isopropyl-4-[3′-(methylsulfonyl)biphenyl-3-yl]-8-(trifluoromethyl)quinazoline

### Step 1: 4-(3-bromophenyl)-2-isopropyl-8-(trifluoromethyl)quinazoline

Prepared as in Example 31 except using isobutyronitrile. MS (ES) m/z 394.6; HRMS: calcd for C₁₈H₁₄BrF₃N₂+H⁺, 395.03652; found (ESI, [M+H]⁺Calc'd), 395.0365.

### Step 2: 2-isopropyl-4-[3′-(methylsulfonyl)biphenyl-3-yl]-8-(trifluoromethyl)quinazoline

Prepared according to a procedure similar to that described in Example 37 except using 4-(3-bromophenyl)-2-isopropyl-8-(trifluoro-methyl)quinazoline and 3-(methanesulfonyl)benzeneboronic acid. MS (ES) m/z 470.7.

### Example 43

### 4-[3′-(methylsulfonyl)biphenyl-3-yl]-2-phenyl-8-(trifluoromethyl)quinazoline

### Step 1: 4-(3-bromophenyl)-2-phenyl-8-(trifluoromethyl)quinazoline

A stirred mixture of (3-bromophenyl)[2-fluoro-3-(trifluoromethyl)phenyl]methanone (0.30 g, 0.86 mmol), benzamidine (0.18 g, 1.11 mmol), and cesium carbonate (0.841 g, 2.58 mmol) in DMF (8 mL) was heated at 120° C. under a nitrogen atmosphere. After 8 h, the mixture was poured into water (15 mL), brine (3 mL), and extracted with ethyl acetate (3×10 mL). The combined extracts were dried (MgSO₄) and concentrated in vacuo. Chromatography on silica gel using a 30:70 to 60:40 EtOAc:Hex gradient gave the title compound as semi-solid (319 mg).

### Step 2: 4-[3′-(methylsulfonyl)biphenyl-3-yl]-2-phenyl-8-(trifluoromethyl)quinazoline

Prepared according to a procedure similar to that described in Example 37 except using 4-(3-bromophenyl)-2-phenyl-8-(trifluoromethyl)quinazoline and 3-(methanesulfonyl)benzeneboronic acid. MS (ES) m/z 504.5.

### Example 44

### 2-methyl-2-[4-({3-[8-(trifluoromethyl)quinazolin-4-yl]phenoxy}methyl)phenyl]propanoic acid

A stirred mixture of 3-(8-(trifluoromethyl)quinazolin-4-yl)phenol (145 mg, 0.50 mmol), ethyl 2-(4-(bromomethyl)phenyl)-acetate (167 mg, 0.65 mmol), and cesium carbonate (489 mg, 1.50 mmol) in dichloromethane (5 mL) was stirred at 20° C. After 7 d, the reaction was treated with dichloromethane (10 mL), filtered through a pad of MgSO₄, and concentrated in vacuo. The residue was chromatographed with a 20/80 to 40/60 ethyl acetate/hexane gradient to afford the title compound as an oil (86 mg). This material was dissolved in THF (2.0 mL) and treated with 1.0 M aqueous LiOH (0.8 mL) at 20° C. for 3 d. The reaction was treated with 1.0 M aqueous HCl (1 mL) and extracted with dichloromethane (3×5 mL). The combined organic extracts were dried (MgSO₄) and concentrated in vacuo. Chromatography on silica gel (50/50 to 100/0 ethyl acetate/hexane gradient) gave the title compound as a white solid (68 mg, R_{f}˜0.20 in ethyl acetate). MS (ES) m/z 466.9, HRMS: calcd for C₂₆H₂₁F₃N₂O₃+H⁺, 467.15770; found (ESI, [M+H]⁺), 467.1567.

### Example 45

### [4-({3-[8-(trifluoromethyl)quinazolin-4-yl]phenoxy}methyl)phenyl]acetic acid

Prepared according to a procedure similar to that described in Example 44, except alkylating with methyl 2-(4-(bromomethyl)phenyl)-2-methylpropanoate. MS (ES) m/z 438.9; HRMS: calcd for C₂₄H₁₇F₃N₂O₃+H⁺, 439.12640; found (ESI, [M+H]⁺), 439.1285.

### Example 46

### 4-chloro-8-(trifluoromethyl)quinazoline

A slurry of 3-trifluoromethylanthranilic acid (1.8 g, 8.8 mmol) was heated in formamide (5 mL) at 135° C. for 45 min, then at 175° C. for 70 min (gas evolution). The mixture was cooled and dissolved in MeOH (40 mL), water (30 mL) was added and the precipitate was filtered (670 mg). The filtrate was extracted with chloroform (4×25 mL) and the combined organic extracts washed with brine (20 mL) and dried over Na₂SO₄. After concentration in vacuo, the solid residue (620 mg) was heated in POCl₃ (10 mL) at 100° C. for 6 h. Similarly, the precipitated product (670 mg) was heated in POCl₃ (6 mL) for 4 h at 100° C. Each solution was poured into ice-water (100 mL) and the mixture was extracted with EtOAc (100 mL). The organic layers for each reaction were combined, and the solutions were washed with 2N aqueous Na₂CO₃ (10×20 mL). The organic layer was dried (Na₂SO₄) and concentrated. The residue (700 mg) was used without further purification. For analysis purposes, a portion of this sample was purified by silica gel chromatography, eluting with 0:100 to 10:90 E:H gradient, yielding the title compound as a white solid. ¹H NMR (400 MHz; CDCl₃) δ 9.16 (1H, s), 8.48 (1H, d, J=8.4 Hz), 8.29 (1H, d, J=7.4 Hz), 7.77 (1H, t, J=7.9 Hz).

### Example 47

### 4-(2-chloro-5-methoxyphenyl)-8-(trifluoromethyl)quinazoline

A stream of nitrogen gas was bubbled through a mixture of 4-chloro-8-(trifluoromethyl)quinazoline (660 mg, 2.83 mmol), 2-chloro-5-methoxyphenylboronic acid (723 mg, 4.26 mmol), 2M aqueous Na₂CO₃ (4.25 mL, 8.5 mmol) in dimethoxyethane (8 mL) for 10 min. Tetrakis-triphenylphosphine palladium (168 mg, 0.14 mmol) was added and the mixture was stirred at 80° C. for 6 h. The suspension was cooled and poured into a mixture of EtOAc (60 mL) and water (30 mL). The layers were separated and the organic layer was further washed with aqueous NaHCO₃ (10 mL), water (10 mL), and brine (20 mL). The organic layer was dried with Na₂SO₄ and concentrated in vacuo. The residue was purified by silica gel chromatography eluting with a gradient of 0:100 to 20:80 E:H to afford white foamy solid (640 mg). MS (ES) m/z 338.7; HRMS: calcd for C₁₆H₁₀ClF₃N₂O+H⁺, 339.05065; found (ESI, [M+H]⁺ Obs'd), 339.0510.

### Example 48

### 4-chloro-3-[8-(trifluoromethyl)quinazolin-4-yl]phenol

A mixture of 4-(2-chloro-5-methoxyphenyl)-8-(trifluoromethyl)quinazoline (540 mg, 1.6 mmol) and solid pyridine hydrochloride (7 g) was heated to 200° C., during which it became a homogenous solution. After 1.5 h, the reaction was poured into stirred water (60 mL)/EtOAc (80 mL) and the layers were separated. The organic layer was further washed with 5% citric acid (2×20 mL), aqueous NaHCO₃ (10 mL), and brine (20 mL). The organic layer was dried with Na₂SO₄ and concentrated in vacuo. The residue was purified by SiO₂ chromatography using a gradient of 0:100 to 30:70 E:H to afford an white solid (420 mg). MS (ES) m/z 324.8; HRMS: calcd for C₁₅H₈ClF₃N₂O+H⁺, 325.03500; found (ESI, [M+H]⁺ Obs'd), 325.0355.

### Example 49

### 4-{2-chloro-5-[3-(methylsulfonyl)phenoxy]phenyl}-8-(trifluoromethyl)quinazoline

A mixture of 4-chloro-3-[8-(trifluoromethyl)quinazolin-4-yl]phenol (from Example 48, 100 mg, 0.31 mmol), Cs₂CO₃ (203 mg, 0.62 mmol), and 1-fluoro-3-(methylsulfonyl)benzene (80 mg, 0.46 mmol) in dimethylacetamide (2 mL) was heated at 150° C. for 24 h. The reaction was cooled and diluted with EtOAc (40 mL) and water (20 mL). The layers were separated and the organic layer was washed water (4×20 mL), and brine (20 mL). The organic layer was dried with Na₂SO₄ and concentrated in vacuo. The residue was purified by SiO₂ chromatography eluting with a gradient of 0:100 to 30:70 E:H to afford the title compound as a white foamy solid (49 mg). MS (ES) m/z 478.7; HRMS: calcd for C₂₂H₁₄ClF₃N₂O₃S+H⁺, 479.04385; found (ESI, [M+H]⁺ Obs'd), 479.0445.

Compounds below were prepared in a similar fashion, using the appropriate halogenated arylsulfones, varying reaction times up to 24 or 48 h.

### Example 50

### 4-{2-chloro-5-[3-(ethylsulfonyl)phenoxy]phenyl}-8-(trifluoromethyl)quinazoline

Prepared according to a procedure similar to that described in Example 3 except using 3-(fluorophenyl)-ethylsulfone. MS (ES) m/z 492.8; HRMS: calcd for C₂₃H₁₆ClF₃N₂O₃S+H⁺, 493.05950; found (ESI, [M+H]⁺ Obs'd), 493.0596.

### Example 51

### 4-{2-chloro-5-[3-(isopropylsulfonyl)phenoxy]phenyl}-8-(trifluoromethyl)quinazoline

Prepared according to a procedure similar to that described in Example 3 except using 3-(fluorophenyl)-isopropylsulfone. MS (ES) m/z 506.6; HRMS: calcd for C₂₄H₁₈ClF₃N₂O₃S+H⁺, 507.07515; found (ESI, [M+H]⁺ Obs'd), 507.0752.

### Example 52

### 4-{2-fluoro-5-[3-(methylsulfonyl)phenoxy]phenyl}-8-(trifluoromethyl)quinazoline

### Step 1: 4-(2-fluoro-5-methoxyphenyl)-8-(trifluoromethyl)quinazoline

Prepared according to a similar procedure to that described in Example 47 except using 2-fluoro-5-methoxyphenylboronic acid. MS (ES) m/z 323.0; HRMS: calcd for C₁₆H₁₀F₄N₂O+H⁺, 323.08020; found (ESI, [M+H]⁺ Obs'd), 323.0804.

### Step 2: 4-fluoro-3-[8-(trifluoromethyl)quinazolin-4-yl]phenol

Prepared according to a similar procedure to that described in Example 48 using 4-(2-fluoro-5-methoxyphenyl)-8-(trifluoromethyl)quinazoline. MS (ES) m/z 309.1; HRMS: calcd for C₁₅H₈F₄N₂O+H⁺, 309.06455; found (ESI, [M+H]⁺ Obs'd), 309.0650.

### Step 3: 4-{2-fluoro-5-[3-(methylsulfonyl)phenoxy]phenyl}-8-(trifluoromethyl)quinazoline

A mixture of 4-fluoro-3-[8-(trifluoromethyl)quinazolin-4-yl]phenol (100 mg, 0.33 mmol), 1-bromo-3-(methylsulfonyl)benzene (113 mg, 0.49 mmol), Cs₂CO₃ (326 mg, 1 mmol), CuI (12 mg, 0.06 mmol), and N,N′-dimethylglycine (16 mg, 0.11 mmol) in dioxane (2 mL) was stirred at 95° C. for 16 h. The reaction was cooled, treated with water, and extracted with EtOAc. The extracts were dried with Na₂SO₄ and concentrated in vacuo. Chromatography on silica gel eluting with EtOAc:Hex gradient of 0:100 to 40:60 afforded the title compound as a white foam-solid. MS (ES) m/z 462.9; HRMS: calcd for C₂₂H₁₄F₄N₂O₃S+H⁺, 463.07340; found (ESI, [M+H]⁺ Obs'd), 463.0741.

### Example 53

### 4-{5-[3-(ethylsulfonyl)phenoxy]-2-fluorophenyl}-8-(trifluoromethyl)quinazoline

Prepared according to a similar procedure to that described in Example 52, Step 3 except using 1-bromo-3-(ethylsulfonyl)benzene. MS (ES) m/z 476.9; HRMS: calcd for C₂₃H₁₆F₄N₂O₃S+H⁺, 477.08905; found (ESI, [M+H]⁺ Obs'd), 477.0896.

### Example 54

### 4-{2-fluoro-5-[3-(isopropylsulfonyl)phenoxy]phenyl}-8-(trifluoromethyl)quinazoline

Prepared according to a similar procedure to that described in Example 52, Step 3 except using 1-bromo-3-(isoproylsulfonyl)benzene. MS (ES) m/z 491.0; HRMS: calcd for C₂₄H₁₈F₄N₂O₃S+H⁺, 491.10470; found (ESI, [M+H]⁺ Obs'd), 491.1056.

### Example 55

### 4-{2-fluoro-5-[3-fluoro-5-(methylsulfonyl)phenoxy]phenyl}-8-(trifluoromethyl)quinazoline

A stirred mixture of 4-fluoro-3-[8-(trifluoromethyl)quinazolin-4-yl]phenol (100 mg, 0.33 mmol), 3,5-difluoro-1-methylsulfonylbenzene (192 mg, 1.00 mmol), and potassium carbonate (138 mg, 1.00 mmol) in dimethylacetamide (2 mL) was heated at 100° C. under a nitrogen atmosphere. After 16 h, the reaction was partitioned between ethyl acetate (40 mL) and water (20 mL). The layers were separated and the organic layer was washed with water (6×20 mL) and brine (20 mL). The combined extracts were dried (Na₂SO₄) and concentrated in vacuo. Chromatography on silica gel (0:100 to 25:75 E:H gradient) gave the title compound as a white solid. MS (ES) m/z 481.0; HRMS: calcd for C₂₂H₁₃F₅N₂O₃S+H⁺, 481.06398; found (ESI, [M+H]⁺ Obs'd), 481.0643.

### Example 56

### 4-(2-fluoro-5-{[3-(methylsulfonyl)benzyl]oxy}phenyl)-8-(trifluoromethyl)quinazoline

Prepared according to a similar procedure to that described in Example 55 except using 1-(bromomethyl)-3-(methylsulfonyl)benzene. MS (ESI) m/z 477.1; HRMS: calcd for C₂₃H₁₆F₄N₂O₃S+H⁺, 477.08905; found (ESI, [M+H]⁺ Obs'd), 477.0895.

### Example 57

### 4-{5-[3-chloro-5-(methylsulfonyl)phenoxy]-2-fluorophenyl}-8-(trifluoromethyl)quinazoline

Prepared according to a similar procedure to that described in Example 55 except using 3,5-dichloro-1-(methylsulfonyl)benzene. MS (ES) m/z 497.0; HRMS: calcd for C₂₂H₁₃ClF₄N₂O₃S+H⁺, 497.03443; found (ESI, [M+H]⁺ Obs'd), 497.0352.

### Example 58

### 4-{2-chloro-5-[3-(methylsulfonyl)phenoxy]phenyl}-2-methyl-8-(trifluoromethyl)quinazoline

### Step 1: 2-methyl-8-(trifluoromethyl)-4H-3,1-benzoxazin-4-one

A mixture of 3-trifluoromethyl-2-aminobenzoic acid (2.15 g, 10.0 mmol) and acetic anhydride (20 mL) was heated at 130° C. for 18 h. The volatile components were removed in vacuo and the residue was used without further purification. MS (ES) m/z 230.1.

### Step 2: 2-methyl-8-(trifluoromethyl)quinazolin-4(3H)-one

A mixture of 2-methyl-8-(trifluoromethyl)-4H-3,1-benzoxazin-4-one (2.00 g, 8.7 mmol) and acetamide (25 g) was heated at 175° C. for 3 h. The mixture was cooled to rt and partitioned between EtOAc (150 mL) and water (100 mL). The organic layer was washed with water (5×50 mL), brine (50 mL), was dried over Na₂SO₄ and concentrated. Chromatography on silica gel eluting with EtOAc:Hex gradient of 0:100 to 40:60 afforded the title compound as a white solid. MS (ES) m/z 229.0; HRMS: calcd for C₁₀H₇F₃N₂O+H⁺, 229.05832; found (ESI, [M+H]⁺ Obs'd), 229.0591.

### Step 3: 2-methyl-8-(trifluoromethyl)quinazolin-4-yl trifluoromethanesulfonate

Trifluoromethanesulfonic anhydride (224 μL, 1.33 mmol) was added over 1 min to a 0° C. solution of 3-[2-methyl-4-(trifluoromethyl)-1H-benzimidazol-1-yl]phenol (354 mg, 1.21 mmol) and diisopropylethylamine (234 μL, 1.82 mmol) in DCM (6 mL). The solution was stirred for 2 h during which it warmed to rt. The reaction was poured into a mixture of EtOAc (40 mL) and citric acid (10 mL) and the layers were separated. The organic layer was washed with citric acid (10 mL), NaHCO₃ (10 mL), and brine (20 mL). The solution was dried over Na₂SO₄, concentrated, and purified by chromatography on SiO₂ eluting with a 0:100 to 10:90 EtOAc:Hex gradient. The product was isolated as a white solid and was used without further purification.

### Step 4: 4-(2-chloro-5-methoxyphenyl)-2-methyl-8-(trifluoromethyl)quinazoline

A mixture of 2-methyl-8-(trifluoromethyl)quinazolin-4-yl trifluoromethanesulfonate (2.7 g, 7.5 mmol), 2-chloro-5-methoxyphenylboronic acid (1.6 g, 9.4 mmol), K₃PO₄ (4.0 g, 18.8 mmol) and Pd(PPh₃)₄ (433 mg, 0.4 mmol) in dioxane (25 mL) was heated at 100° C. for 2 h. The mixture was poured into a mixture of EtOAc (100 mL) and water (70 mL) and the layers were separated. The organic layer was washed with NaHCO₃ (2×50 mL), water (50 mL), and brine (70 mL). The solution was concentrated and the residue was redissolved in ˜15 mL of DCM. The solution was filtered (900 mg of 2-methyl-8-(trifluoromethyl)quinazolin-4(3H)-one was recovered) and the supernatent was added to a column of SiO₂ which was eluted with a gradient of 0:100 to 20:80 EtOAc:Hex. The product was isolated as a white foam. MS (ES) m/z 352.9; HRMS: calcd for C₁₇H₁₂ClF₃N₂O+H⁺, 353.06630; found (ESI, [M+H]⁺ Obs'd), 353.0668.

### Step 5: 4-chloro-3-[2-methyl-8-(trifluoromethyl)quinazolin-4-yl]phenol

Prepared according to a similar procedure to that described in Example 48 using 4-(2-chloro-5-methoxyphenyl)-2-methyl-8-(trifluoromethyl)quinazoline. MS (ES) m/z 338.9; HRMS: calcd for C₁₆H₁₀ClF₃N₂O+H⁺, 339.05065; found (ESI, [M+H]⁺ Obs'd), 339.0513

### Step 6: 4-{2-chloro-5-[3-(methylsulfonyl)phenoxy]phenyl}-2-methyl-8-(trifluoromethyl)quinazoline

Prepared according to a similar procedure to that described in Example 52, Step 3. MS (ES) m/z 492.9; HRMS: calcd for C₂₃H₁₆ClF₃N₂O₃S+H⁺, 493.05950; found (ESI, [M+H]⁺ Obs'd), 493.0595.

### Example 59

### 4-{2-chloro-5-[3-(ethylsulfonyl)phenoxy]phenyl}-2-methyl-8-(trifluoromethyl)quinazoline

Prepared according to a similar procedure to that described in Example 52, Step 3, except using 1-bromo-3-(ethylsulfonyl)benzene. MS (ES) m/z 506.9; HRMS: calcd for C₂₄H₁₈ClF₃N₂O₃S+H⁺, 507.07515; found (ESI, [M+H]⁺ Obs'd), 507.0752.

### Example 60

### 4-{2-chloro-5-[3-(isopropylsulfonyl)phenoxy]phenyl}-2-methyl-8-(trifluoromethyl)quinazoline

Prepared according to a similar procedure to that described in Example 52, Step 3, except using 1-bromo-3-(isopropyl)sulfonylbenzene. MS (ES) m/z 520.9; HRMS: calcd for C₂₅H₂₀ClF₃N₂O₃S+H⁺, 521.09080; found (ESI, [M+H]⁺ Obs'd), 521.0909.

### Example 61

### 4-{2-chloro-5-[3-fluoro-5-(methylsulfonyl)phenoxy]phenyl}-2-methyl-8-(trifluoromethyl)quinazoline

Prepared according to a similar procedure to that described in Example 55. MS (ES) m/z 510.8; HRMS: calcd for C₂₃H₁₅ClF₄N₂O₃S+H⁺, 511.05008; found (ESI, [M+H]⁺ Obs'd), 511.0499.

### Example 62

### 4-{2-chloro-5-[3-(ethylsulfonyl)-5-fluorophenoxy]phenyl}-2-methyl-8-(trifluoromethyl)quinazoline

Prepared according to a similar procedure to that described in Example 55 except using 3,5-difluoro-1-(ethylsulfonyl)benzene. MS (ES) m/z 524.9; HRMS: calcd for C₂₄H₁₇ClF₄N₂O₃S+H⁺, 525.06573; found (ESI, [M+H]⁺ Obs'd), 525.0657.

### Example 63

### 4-(2-chloro-5-{[3-(methylsulfonyl)benzyl]oxy}phenyl)-2-methyl-8-(trifluoromethyl)quinazoline

Prepared according to a similar procedure to that described in Example 55 except using 1-(bromomethyl)-3-(methylsulfonyl)benzene. MS (ESI) m/z 507.1; HRMS: calcd for C₂₄H₁₈ClF₃N₂O₃S+H⁺, 507.07515; found (ESI, [M+H]⁺ Obs'd), 507.0763.

### Example 64

### 8-chloro-4-{2-fluoro-5-[3-fluoro-5-(methylsulfonyl)phenoxy]phenyl}quinazoline

### Step 1: 8-chloroquinazolin-4(3H)-one

A slurry of 2-amino-3-chlorobenzoic acid (3.26 g, 8.8 mmol) was heated in formamide (5 mL) at 135° C. for 90 min, then at 175° C. for 90 min. The mixture was cooled to rt and poured into water (150 mL). The solid was collected and washed with 0.1N NH₄OH (100 mL). The off-white solid was used without further purification. MS (ESI) m/z 179.2; HRMS: calcd for C₈H₅ClN₂O+H⁺, 181.01632; found (ESI, [M+H]⁺ Obs'd), 181.0164.

### Step 2: 4,8-dichloroquinazoline

A suspension of 8-chloroquinazolin-4(3H)-one (2.74 g, 15.2 mmol) and DMF (200 μL) was heated in thionyl chloride (80 mL) at 72° C. for 8 h, with venting, during which the solution became homogeneous. The solution was slowly added into vigorously stirred ice/water (gas evolution!) and the resulting solid was collected. The solid was washed with water and dried to yield an off-white solid that was used without further purification.

### Step 3: 8-chloro-4-(2-fluoro-5-methoxyphenyl)quinazoline

Prepared according to a similar procedure to that described in Example 47 except using 2-fluoro-5-methoxyphenylboronic acid. MS (ESI) m/z 289.0; HRMS: calcd for C₁₅H₁₀ClFN₂O+H⁺, 289.05384; found (ESI, [M+H]⁺ Obs'd), 289.0541.

### Step 4: 3-(8-chloroquinazolin-4-yl)-4-fluorophenol

Prepared according to a similar procedure to that described in Example 48 from 8-chloro-4-(2-fluoro-5-methoxyphenyl)quinazoline. MS (ESI) m/z 275.0; HRMS: calcd for C₁₄H₈ClFN₂O+H⁺, 275.03819; found (ESI, [M+H]⁺ Obs'd), 275.0387.

### Step 5: 8-chloro-4-{2-fluoro-5-[3-fluoro-5-(methylsulfonyl)phenoxy]phenyl}quinazoline

Prepared according to a similar procedure to that described in Example 55. MS (ESI) m/z 447.1; HRMS: calcd for C₂₁H₁₃ClF₂N₂O₃S+H⁺, 447.03762; found (ESI, [M+H]⁺ Obs'd), 447.0375.

### Example 65

### 8-chloro-4-{5-[3-(ethylsulfonyl)-5-fluorophenoxy]-2-fluorophenyl}quinazoline

Prepared according to a similar procedure to that described in Example 55 except using 3,5-difluoro-1-(ethylsulfonyl)benzene. MS (ESI) m/z 461.1; HRMS: calcd for C₂₂H₁₅ClF₂N₂O₃S+H⁺, 461.05327; found (ESI, [M+H]⁺ Obs'd), 461.0533.

### Example 66

### 8-chloro-4-(2-fluoro-5-{[3-(methylsulfonyl)benzyl)oxy]phenyl}quinazoline

Prepared according to a similar procedure to that described in Example 55. 5. MS (ESI) m/z 443.1; HRMS: calcd for C₂₂H₁₆ClFN₂O₃S+H⁺, 443.06269; found (ESI, [M+H]⁺ Obs'd), 443.0630.

### Example 67

### 8-chloro-4-{2-fluoro-5-[3-(ethylsulfonyl)phenoxy]phenyl}quinazoline

Prepared according to a similar procedure to that described in Example 52, Step 3, except using 1-bromo-3-(ethylsulfonyl)benzene. MS (ESI) m/z 443.1; HRMS: calcd for C₂₂H₁₆ClFN₂O₃S+H⁺, 443.06269; found (ESI, [M+H]⁺ Obs'd), 443.0630.

### Example 68

### 8-chloro-4-(2-fluoro-5-{[3-(1-methylethyl)sulfonyl]phenoxy}phenyl)quinazoline

Prepared according to a similar procedure to that described in Example 52, Step 3, except using 1-bromo-3-(isopropylsulfonyl)benzene. MS (ESI) m/z 457.1; HRMS: calcd for C₂₃H₁₈ClFN₂O₃S+H⁺, 457.07834; found (ESI, [M+H]⁺ Obs'd), 457.0781.

### Example 69

### 8-chloro-4-[2-fluoro-5-(3-{[3-(tetrahydro-2H-pyran-2-yloxy)propyl]sulfonyl}phenoxy)phenyl]quinazoline

Prepared according to a similar procedure to that described in Example 52, Step 3, except using 2-{3-[(3-bromophenyl)sulfonyl]propoxy}tetrahydro-2H-pyran. HRMS: calcd for C₂₈H₂₆ClFN₂O₅S+H⁺, 557.13077; found (ESI, [M+H]⁺ Obs'd), 557.1302.

### Example 70

### 3-[3-(8-chloroquinazolin-4-yl)-4-fluorophenoxy]benzonitrile

Prepared according to a similar procedure to that described in Example 52, Step 3, except using 3-bromobenzonitrile. MS (ESI) m/z 376.1; HRMS: calcd for C₂₁H₁₁ClFN₃O+H⁺, 376.06474; found (ESI, [M+H]⁺ Obs'd), 376.0648.

### Example 71

### 3-[3-(8-chloroquinazolin-4-yl)-4-fluorophenoxy]benzoic acid

A mixture of 4-fluoro-3-[8-(trifluoromethyl)quinazolin-4-yl]phenol (138 mg, 0.50 mmol), ethyl 3-iodobenzoate (276 mg, 1.00 mmol), Cs₂CO₃ (978 mg, 3.00 mmol), CuI (36 mg, 0.18 mmol), and N,N′-dimethylglycine (48 mg, 0.33 mmol) in dioxane (4 mL) was stirred at 95° C. for 16 h. The reaction was cooled and EtOAc (20 mL) and EtOH (5 mL) was added. The solids were removed by filtration and the supernatant was concentrated. The residue was purified by chromatography on silica gel eluting with EtOAc:Hex gradient of 0:100 to 20:80 to afford the benzoate product as a mixture of methyl and ethyl esters. The residue was dissolved in MeOH and 2M NaOH (2 mL) was added. After 1 h of stirring, 2N HCl was added dropwise with vigorous stirring to pH˜1. The precipitate (title compound) was collected as a white solid. MS (ESI) m/z 395.1; HRMS: calcd for C₂₁H₁₂ClFN₂O₃+395.05932; found (ESI, [M+H]⁺ Obs'd), 395.0595.

### Example 72

### 3-[(3-{4-chloro-3-[2-methyl-8-(trifluoromethyl)quinazolin-4-yl]phenoxy}phenyl)sulfonyl]propan-1-ol

Prepared according to a similar procedure to that described in Example 52, Step 3, using 4-chloro-3-[2-methyl-8-(trifluoromethyl)quinazolin-4-yl]phenol and 3-[4(3-bromophenyl)sulfonyl]propan-1-ol. MS (ESI) m/z 537.1.

### Example 73

### 3-({3-[3-(8-chloroquinazolin-4-yl)-4-fluorophenoxy]phenyl}sulfonyl)propan-1-ol

A mixture of 8-chloro-4-[2-fluoro-5-(3-{[3-(tetrahydro-2H-pyran-2-yloxy)propyl]sulfonyl}phenoxy)phenyl]quinazoline (260 mg, 0.47 mmol) and benzenesulfonic acid (15 mg, 0.10 mmol) was stirred in MeOH (20 mL) at rt for 18 h. The solvent was removed in vacuo and the residue was purified by chromatography on SiO₂ eluting with a 5:95 to 75:25 EtOAc:Hex gradient. The product was isolated as a white foam. MS (ESI) m/z 473.1

### Example 74

### Methyl 3-[3-(8-chloroquinazolin-4-yl)-4-fluorophenoxy]benzoate

A solution of trimethylsilyldiazomethane (1M in DCM; 130 μL, 0.13 mmol) was added to a stirred solution of 3-[3-(8-chloroquinazolin-4-yl)-4-fluorophenoxy]benzoic acid (40 mg, 0.10 mmol) in DCM (3 mL) and MeOH (1 mL). After 15 min, acetic acid (50 μL) was added and the solution was stirred for 15 min. The solvent was evaporated and the residue was purified by chromatography on SiO₂ eluting with a 0:100 to 20:80 EtOAc:Hex gradient. The product was isolated as a white foam. MS (ESI) m/z 409.1.

### Example 75

### 8-chloro-4-{2-fluoro-5-[3-(methylsulfonyl)phenoxy]phenyl}quinazoline

Prepared according to a similar procedure to that described in Example 52, Step 3, except using 3-(8-chloroquinazolin-4-yl)-4-fluorophenol and 1-bromo-3-(methylsulfonyl)benzene. MS (ESI) m/z 429.1; HRMS: calcd for C₂₁H₁₄ClFN₂O₃S+H⁺, 429.04704; found (ESI, [M+H]⁺ Obs'd), 429.0470.

### Example 76

### 2-cyclopropyl-4-[3′-(methylsulfonyl)biphenyl-3-yl]-8-(trifluoromethyl)quinazoline

### Step 1: 4-(3-bromophenyl)-2-cyclopropyl-8-(trifluoromethyl)quinazoline

Prepared as in Example 29, except using cyclopropanecarboximidamide.

### Step 2: 2-cyclopropyl-4-[3′-(methylsulfonyl)biphenyl-3-yl]-8-(trifluoromethyl)quinazoline

Prepared as in Example 37 except using 4-(3-bromophenyl)-2-cyclopropyl-8-(trifluoromethyl)quinazoline and 3-(methanesulfonyl)benzeneboronic acid. MS (ES) m/z 469.1.

### Example 77

### 4-{2-chloro-5-[3-(methylsulfonyl)phenoxy]phenyl}-8-methoxyquinazoline

### Step 1: 8-methoxyquinazolin-4-ol

Prepared in a manner similar to Example 64, Step 1, using 2-amine-3-methoxy-benzoic acid. MS (ESI) m/z 177.1; HRMS: calcd for C₉H₈N₂O₂+H⁺, 177.06585; found (ESI, [M+H]⁺ Obs'd), 177.0660.

### Step 2: 4-chloro-8-methoxyquinazoline

Prepared in a manner similar to Example 64, Step 2, using 8-methoxyquinazolin-4-ol. MS (ESI) m/z 195.0; HRMS: calcd for C₉H₇ClN₂O+H⁺, 195.03197; found (ESI, [M+H]⁺ Obs'd), 195.0320.

### Step 3: 4-chloro-3-[8-methoxyquinazolin-4-yl]-phenol

A stream of nitrogen gas was bubbled through a mixture of 4-chloro-8-methoxyquinazoline (582 mg, 3.00 mmol), 2-chloro-5-hydroxyphenylboronic acid (723 mg, 4.26 mmol), sat'd aqueous NaHCO₃ (10 mL), dimethoxyethane (15 mL), and water (5 mL) for 10 min. Tetrakis-triphenylphosphine palladium (346 mg, 0.30 mmol) was added and the mixture was stirred at 75° C. for 6 h. The suspension was cooled and poured into a mixture of EtOAc (80 mL) and water (50 mL). The layers were separated and the organic layer was further washed with aqueous NaHCO₃ (10 mL), water (10 mL), and brine (20 mL). The organic layer was dried with Na₂SO₄ and concentrated in vacuo. The residue was purified by silica gel chromatography eluting with a gradient of 0:100 to 60:40 E:H to afford the title compound an impure colorless glass (200 mg, contaminated with triphenylphosphine oxide in ˜1:1 molar ratio by ¹H NMR analysis). The material was used in the next step without further purification.

### Step 4: 4-{2-chloro-5-[3-(methylsulfonyl)phenoxy]phenyl}-8-methoxyquinazoline

A mixture of impure 4-chloro-3-[8-methoxyquinazolin-4-yl]phenol (200 mg), 3-(methanesulfonyl)-benzeneboronic acid (140 mg, 0.70 mmol), Cu(OAc)₂ (95 mg, 0.53 mmol), pyridine (169 μL, 2.10 mmol), and molecular sieves (2 g) in dichloromethane (10 mL) was vigorously stirred in a vial open to the atmosphere, for 16 h. The mixture was diluted with EtOAc (50 mL) and the solids were filtered. The filtrate was washed with 10% aqueous citric acid (2×10 mL), Na₂CO₃ (2×10 mL), and brine (20 mL). The organic layer was dried over Na₂SO₄ then was concentrated. The residue was purified by chromatography on silica gel eluting with EtOAc:Hex gradient of 0:100 to 50:50 to afford the title compound as a white foam solid. HRMS: calcd for C₂₂H₁₇ClN₂O₄S+H⁺, 441.06703; found (ESI, [M+H]⁺ Obs'd), 441.0677.

### Example 78

### 8-chloro-4-{2-chloro-5-[3-(methylsulfonyl)phenoxy]phenyl}quinazoline

### Step 1: 8-chloro-4-(2-chloro-5-methoxyphenyl)quinazoline

Prepared according to a similar procedure to that described in Example 47 using 4,8-dichloroquinazoline and 2-chloro-5-methoxyphenylboronic acid. MS (ESI) m/z 305.0; HRMS: calcd for C₁₅H₁₀Cl₂N₂O+H⁺, 305.02429; found (ESI, [M+H]⁺ Obs'd), 305.0247.

### Step 2: 4-chloro-3-(8-chloroquinazolin-4-yl)phenol

Prepared according to a similar procedure to that described in Example 48, from 8-chloro-4-(2-chloro-5-methoxyphenyl)quinazoline. MS (ESI) m/z 291.0; HRMS: calcd for C₁₄H₈Cl₂N₂O+H⁺, 291.00864; found (ESI, [M+H]⁺ Obs'd), 291.0089.

### Step 3: 8-chloro-4-{2-chloro-5-[3-(methylsulfonyl)phenoxy]phenyl}quinazoline

Prepared according to a similar procedure to that described in Example 52, step 3, from 4-chloro-3-(8-chloroquinazolin-4-yl)phenol and 1-bromo-3-(methylsulfonyl)benzene. MS (ESI) m/z 445.1; HRMS: calcd for C₂₁H₁₄Cl₂N₂O₃S+H⁺, 445.01749; found (ESI, [M+H]⁺ Obs'd), 445.0171.

### Example 79

### 3-({3-[4-chloro-3-(8-chloroquinazolin-4-yl)phenoxy]phenyl}sulfonyl)propan-1-ol

Prepared according to a similar procedure to that described in Example 52, step 3, from 4-chloro-3-(8-chloroquinazolin-4-yl)phenol and 3-[(3-bromophenyl)sulfonyl]propan-1-ol. MS (ESI) m/z 489.1; HRMS: calcd for C₂₃H₁₈Cl₂N₂O₄S+H⁺, 489.04371; found (ESI, [M+H]⁺ Obs'd), 489.0435.

### Example 80

### 8-chloro-4-{2-chloro-5-[3-(ethylsulfonyl)phenoxy]phenyl}quinazoline

Prepared according to a similar procedure to that described in Example 52, step 3, from 4-chloro-3-(8-chloroquinazolin-4-yl)phenol and 1-bromo-3-(ethylsulfonyl)benzene. MS (ESI) m/z 459.1; HRMS: calcd for C₂₂H₁₆Cl₂N₂O₃S+H⁺, 459.03314; found (ESI, [M+H]⁺), 459.0328.

### Example 81

### 8-chloro-4-{2-chloro-5-[3-(isopropylsulfonyl)phenoxy]phenyl}quinazoline

Prepared according to a similar procedure to that described in Example 52, step 3, from 4-chloro-3-(8-chloroquinazolin-4-yl)phenol and 1-bromo-3-(isopropylsulfonyl)benzene. MS (ESI) m/z 473.1; HRMS: calcd for C₂₃H₁₈Cl₂N₂O₃S+H⁺, 473.04879; found (ESI, [M+H]⁺ Obs'd), 473.0492.

### Example 82

### 4-({3-[4-chloro-3-(8-chloroquinazolin-4-yl)phenoxy]phenyl}sulfonyl)butan-1-ol

Prepared according to a similar procedure to that described in Example 52, step 3, from 4-chloro-3-(8-chloroquinazolin-4-yl)phenol and 3-[(3-bromophenyl)sulfonyl]butan-1-ol. HRMS: calcd for C₂₄H₂₀Cl₂N₂O₄S+H⁺, 503.05936; found (ESI, [M+H]⁺ Obs'd), 503.0593.

### Example 83

### 4-[(3-{4-chloro-3-[8-(trifluoromethyl)quinazolin-4-yl]phenoxy}phenyl)sulfonyl]butan-1-ol

Prepared according to a similar procedure to that described in Example 52, step 3, from 4-chloro-3-[8-(trifluoromethyl)quinazolin-4-yl]phenol and 3-[(3-bromophenyl)sulfonyl]butan-1-ol. HRMS: calcd for C₂₅H₂₀ClF₃N₂O₄S+H⁺, 537.08572; found (ESI, [M+H]⁺ Obs'd), 537.0860.

### Example 84

### 3-[(3-{4-chloro-3-[8-(trifluoromethyl)quinazolin-4-yl]phenoxy}phenyl)sulfonyl]propan-1-ol

Prepared according to a similar procedure to that described in Example 52, step 3, from 4-chloro-3-[8-(trifluoromethyl)quinazolin-4-yl]phenol and 3-[(3-bromophenyl)sulfonyl]propan-1-ol. MS (ESI) m/z 523.1; HRMS: calcd for C₂₄H₁₈ClF₃N₂O₄S+H⁺, 523.07006; found (ESI, [M+H]⁺ Obs'd), 523.0701.

### Example 85

### 4-(2-chloro-5-{[3-(methylsulfonyl)benzyl]oxy}phenyl)-8-(trifluoromethyl)quinazoline

Prepared according to a similar procedure to that described in Example 55 from 4-chloro-3-[8-(trifluoromethyl)quinazolin-4-yl]phenol and 1-(bromomethyl)-3-(methylsulfonyl)benzene. MS (ESI) m/z 493.1; HRMS: calcd for C₂₃H₁₆ClF₃N₂O₃S+H⁺, 493.05950; found (ESI, [M+H]⁺ Obs'd), 493.0599.

### Example 86

### 4-{2-chloro-5-[3-fluoro-5-(methylsulfonyl)phenoxy]phenyl}-8-(trifluoromethyl)quinazoline

Prepared according to a similar procedure to that described in Example 55 from 4-chloro-3-[8-(trifluoromethyl)quinazolin-4-yl]phenol and 1,3-difluoro-5-(methylsulfonyl)benzene. MS (ESI) m/z 497.1; HRMS: calcd for C₂₂H₁₃ClF₄N₂O₃S+H⁺, 497.03443; found (ESI, [M+H]⁺ Obs'd), 497.0348.

### Example 87

### 4-{2-chloro-5-[3-(ethylsulfonyl)-5-fluorophenoxy]phenyl}-8-(trifluoromethyl)quinazoline

Prepared according to a similar procedure to that described in Example 55 from 4-chloro-3-[8-(trifluoromethyl)quinazolin-4-yl]phenol and 1,3-difluoro-5-(ethylsulfonyl)benzene. MS (ESI) m/z 511.1; HRMS: calcd for C₂₃H₁₅ClF₄N₂O₃S+H⁺, 511.05008; found (ESI, [M+H]⁺ Obs'd), 511.0506.

### Example 88

### 4-{2-chloro-5-[3-chloro-5-(methylsulfonyl)phenoxy]phenyl}-8-(trifluoromethyl)quinazoline

Prepared according to a similar procedure to that described in Example 55 from 4-chloro-3-[8-(trifluoromethyl)quinazolin-4-yl]phenol and 1,3-dichloro-5-(methylsulfonyl)benzene, but heating at 140° C. MS (ESI) m/z 513.1; HRMS: calcd for C₂₂H₁₃Cl₂F₃N₂O₃S+H⁺, 513.00488; found (ESI, [M+H]⁺ Obs'd), 513.0051.

### Example 89

### 8-chloro-4-(2-chloro-5-{[3-(methylsulfonyl)benzyl]oxy}phenyl)quinazoline

Prepared according to a similar procedure to that described in Example 55 from 4-chloro-3-(8-chloroquinazolin-4-yl)phenol and 1-(bromomethyl)-3-(methylsulfonyl)benzene. MS (ESI) m/z 459.1; HRMS: calcd for C₂₂H₁₆Cl₂N₂O₃S+H⁺, 459.03314; found (ESI, [M+H]⁺ Obs'd), 459.0334.

### Example 90

### 8-chloro-4-{2-chloro-5-[3-fluoro-5-(methylsulfonyl)phenoxy]phenyl}quinazoline

Prepared according to a similar procedure to that described in Example 55 from 4-chloro-3-(8-chloroquinazolin-4-yl)phenol and 1,3-difluoro-5-(methylsulfonyl)benzene. MS (ESI) m/z 463.1; HRMS: calcd for C₂₁H₁₃Cl₂FN₂O₃S+H⁺, 463.00807; found (ESI, [M+H]⁺ Obs'd), 463.0084.

### Example 91

### 8-chloro-4-{2-chloro-5-[3-(ethylsulfonyl)-5-fluorophenoxy]phenyl}quinazoline

Prepared according to a similar procedure to that described in Example 55 from 4-chloro-3-(8-chloroquinazolin-4-yl)phenol and 1,3-difluoro-5-(ethylsulfonyl)benzene. MS (ESI) m/z 477.1; HRMS: calcd for C₂₂H₁₅O₂FN₂O₃S+H⁺, 477.02372; found (ESI, [M+H]⁺ Obs'd), 477.0238.

### Example 92

### 8-chloro-4-{2-chloro-5-[3-chloro-5-(methylsulfonyl)phenoxy]phenyl}quinazoline

Prepared according to a similar procedure to that described in Example 55 from 4-chloro-3-(8-chloroquinazolin-4-yl)phenol and 1,3-dichloro-5-(methylsulfonyl)benzene. MS (ESI) m/z 479.0; HRMS: calcd for C₂₁H₁₃Cl₃N₂O₃S+H⁺, 478.97852; found (ESI, [M+H]⁺ Obs'd), 478.9785.

### Example 93

### 3-[(3-{4-fluoro-3-[8-(trifluoromethyl)quinazolin-4-yl]phenoxy}phenyl)sulfonyl]propan-1-ol

Prepared according to a similar procedure to that described in Example 52, step 3, from 4-fluoro-3-[8-(trifluoromethyl)quinazolin-4-yl]phenol and 3-[(3-bromophenyl)sulfonyl]propan-1-ol. MS (ESI) m/z 507.2; HRMS: calcd for C₂₄H₁₈F₄N₂O₄S+H⁺, 507.09962; found (ESI, [M+H]⁺ Obs'd), 507.1000.

### Example 94

### 4-[(3-{4-fluoro-3-[8-(trifluoromethyl)quinazolin-4-yl]phenoxy}phenyl)sulfonyl]butan-1-ol

Prepared according to a similar procedure to that described in Example 52, step 3, from 4-fluoro-3-[8-(trifluoromethyl)quinazolin-4-yl]phenol and 3-[(3-bromophenyl)sulfonyl]butan-1-ol. MS (ESI) m/z 521.2; HRMS: calcd for C₂₅H₂₀F₄N₂O₄S+H⁺, 521.11527; found (ESI, [M+H]⁺ Obs'd), 521.1155.

### Example 95

### 4-({3-[3-(8-chloroquinazolin-4-yl)-4-fluorophenoxyl]phenyl}sulfonyl)butan-1-ol

Prepared according to a similar procedure to that described in Example 52, step 3, from 4-fluoro-3-(8-chloroquinazolin-4-yl)phenol and 3-[(3-bromophenyl)sulfonyl]butan-1-ol. MS (ESI) m/z 487.1; HRMS: calcd for C₂₄H₂₀ClFN₂O₄S+H⁺, 487.08891; found (ESI, [M+H]⁺ Obs'd), 487.0892.

### Example 96

### 3-[(3-{4-chloro-3-[2-methyl-8-(trifluoromethyl)quinazolin-4-yl]phenoxy}phenyl)sulfonyl]butan-1-ol

Prepared according to a similar procedure to that described in Example 52, Step 3, using 4-chloro-3-[2-methyl-8-(trifluoromethyl)quinazolin-4-yl]phenol and 3-[(3-bromophenyl)sulfonyl]butan-1-ol. MS (ESI) m/z 551.2.

### Example 97

### 4-(2-fluoro-5-{3-[(methylsulfonyl)methyl]phenoxy}phenyl)-8-(trifluoromethyl)quinazoline

The title compound was prepared according to the procedure of Example 52, step 3 except using 4-fluoro-3-[8-(trifluoromethyl)quinazolin-4-yl]phenol and 1-bromo-3-[(methylsulfonyl)methyl]benzene as the substrates. MS (ESI) m/z 477.1. HRMS: calcd for C₂₃H₁₆F₄N₂O₃S+H⁺, 477.08905; found (ESI, [M+H]⁺ Obs'd), 477.088.

### Example 98

### Preparation of Sulfone Intermediates

### Intermediate 1

### 1-(ethylsulfonyl)-3-fluorobenzene

A stirred mixture of 3-fluorobenzenesulfonyl chloride (0.973 g, 5.00 mmol), sodium bicarbonate (0.84 g, 10.0 mmol), and sodium sulfite (1.16 g, mmol) was heated in water (7 mL) at 95-100° C. for 1 h under nitrogen. The reaction was cooled to ˜50° C., treated with (nBu)₄NBr (100 mg) and ethyl iodide (2.5 mL) and heated at 70° C. for 18 h. The reaction was cooled, treated with water (10 mL) and extracted with dichloromethane (3×15 mL). The extracts were dried with MgSO₄ and concentrated in vacuo. Chromatography on silica gel eluting with an ethyl acetate:hexane gradient of 10:90 to 40/60 gave the title compound as a colorless oil (878 mg). MS (ES) m/z 189.0.

### Intermediates 2 to 11; 13-14

Prepared according to a procedure similar to that described for Intermediate 1, using the appropriate halogenated arylsulfonylchloride and alkylating agent R-LG, and eluting with an appropriate eluent.

### Intermediate 2

1-fluoro-3-(methylsulfonyl)benzene; MS (ES) m/z 175.0.

### Intermediate 3

1,3-difluoro-5-(methylsulfonyl)benzene; MS (EI) m/z 192.

### Intermediate 4

1-fluoro-3-[(3-methylbutyl)sulfonyl]benzene; MS (ES) m/z 231.0.

### Intermediate 5

1-fluoro-3-(isobutylsulfonyl)benzene; MS (ES) m/z 217.0.

### Intermediate 6

1-fluoro-3-(propylsulfonyl)benzene; MS (ES) m/z 203.0.

### Intermediate 7

3-[(3-fluorophenyl)sulfonyl]propan-1-ol; MS (ES) m/z 218.9; HRMS: calcd for C₉H₁₁FO₃S+H⁺, 219.04857; found (ESI, [M+H]⁺), 219.0475.

### Intermediate 8

1-fluoro-3-(isopropylsulfonyl)benzene; MS (ES) m/z 203.0.

### Intermediate 9

1,3-dichloro-5-(propylsulfonyl)benzene; mp 59-61° C.; MS (ES) m/z 252.9; HRMS: calcd for C₉H₁₀Cl₂O₂S, 251.97785; found (EI, M+.), 251.9776.

### Intermediate 10

1-fluoro-3-[(3-methoxypropyl)sulfonyl]benzene; MS (ES) m/z 233.0; HRMS: calcd for C₁₀H₁₃FO₃S+H⁺, 233.06422; found (ESI, [M+H]⁺), 233.0643.

### Intermediate 11

1-fluoro-4-(propylsulfonyl)benzene; MS (ES) m/z 203.1.

### Preparation of Halogenated Arylsulfones by Thiophenol Alkylation, Oxidation

### Intermediate 8, Second Approach

### 1-fluoro-3-(isopropylsulfonyl)benzene

A stirred mixture of 3-fluorobenzenethiol (3.38 mL, 40.0 mmol), potassium carbonate (11.04 g, 80.0 mmol), and 2-iodopropane (6.00 mL, 60.0 mmol) was heated in acetone (120 mL) at 65-70° C. for 2.5 h under nitrogen. The reaction was cooled, treated with 0.3 M sodium bicarbonate in water (240 mL) and then, in portions, OXONE (61.6 g) and then stirred at ambient temperature for 18 h. The reaction was treated with water (100 mL) and extracted with dichloromethane (2×150 mL). The extracts were dried with MgSO₄ and concentrated in vacuo. Chromatography on silica gel eluting with an ethyl acetate:hexane gradient of 25:75 to 50:50 gave the title compound as a slightly orange liquid (6.21 g). HRMS: calcd for C₉H₁₁FO₂S, 202.04638; found (EI, M+.), 202.0469.

### Intermediates 1, 2, 7, Second Approach

Prepared according to a procedure similar to that described for Intermediate 8, second approach above, using the appropriate halogenated thiophenol and alkylating agent R-LG (where LG was a leaving group such as a bromine, iodide, chloride, or tosylate), and eluting with an appropriate eluent.

### Intermediate 2, Second Approach

1-fluoro-3-(methylsulfonyl)benzene; MS (ES) m/z 175.1

### Intermediate 1, Second Approach

1-(ethylsulfonyl)-3-fluorobenzene; MS (ES) m/z 189.0

### Intermediate 7, Second Approach

3-[(3-fluorophenyl)sulfonyl]propan-1-ol; MS (ES) m/z 218.9

### Intermediate 2, Third Approach

### 1-fluoro-3-(methylsulfonyl)benzene

A stirred mixture of 1-bromo-3-fluorobenzene (10.0 g, 57.1 mmol), sodium methanesulfinate (7.00 g, 68.6 mmol), CuI (1.08 g, 5.71 mmol), L-proline (1.31 g, 11.4 mmol) and sodium hydroxide (0.456 g, 11.4 mmol) was heated in DMSO (135 mL) at 95° C. overnight (˜18 h). The reaction was cooled, diluted with water, and then extracted with ethyl acetate (2×150 mL). The extracts were dried with MgSO₄ and concentrated in vacuo. Chromatography on silica gel eluting with 25/75 ethyl acetate/hexane gave the title compound as a colorless solid (6.21 g). MS (ES) m/z 175.1.

### Intermediates 3, 12 by Third Approach

Prepared according to a procedure similar to that described for Intermediate 8, using the appropriate halogenated thiophenol and alkylating agent R-LG (where LG was a leaving group such as a bromine, iodide, chloride, or tosylate), and eluting with an appropriate eluent.

### Intermediate 12

1-chloro-3-fluoro-5-(methylsulfonyl)benzene; MS (EI) m/z 208

### Intermediate 3

1,3-difluoro-5-(methylsulfonyl)benzene; MS (EI) m/z 192.

### Intermediate 13

### 2-{3-[(3-bromophenyl)sulfonyl]propoxy}tetrahydro-2H-pyran

Prepared according to a procedure similar to that described for Intermediate 8, using the appropriate halogenated thiophenol and alkylating agent R-LG (where LG was a leaving group such as a bromine, iodide, chloride, or tosylate), and eluting with an appropriate eluent. MS (ES) m/z 361.4.

### Intermediate 14

### 1-bromo-3-(ethylsulfonyl)benzene

Prepared according to a procedure similar to that described for Intermediate 8, using 3-bromothiophenol and ethyl iodide, and eluting with an appropriate eluent. MS (ES) m/z 247.9.

### Intermediate 15

### 3-[(3-bromophenyl)sulfonyl]propan-1-ol

Prepared according to a procedure similar to that described for Intermediate 8, using 3-bromothiophenol and 3-bromopropan-1-ol, and eluting with an appropriate eluent. MS (ES) m/z 247.9.

### Intermediate 16

### 4-[(3-bromophenyl)sulfonyl]butan-1-ol

Prepared according to a procedure similar to that described for Intermediate 8, using 3-bromothiophenol and 3-bromobutan-1-ol, and eluting with an appropriate eluent. MS (ESI) m/z 293.0; HRMS: calcd for C₁₀H₁₃BrO₃S+H⁺, 292.98415; found (ESI, [M+H]⁺ Obs'd), 292.9850.

### Example 99

### Biological Testing

Representative compounds of this invention were evaluated in conventional pharmacological test procedures which measured their affinity to bind to LXR and to upregulate the gene ABCA1, which causes cholesterol efflux from atherogenic cells, such as macrophages.

LXR activation can be critical for maintaining cholesterol homeostasis, but its coincident regulation of fatty acid metabolism may lead to increased serum and hepatic triglyceride levels. Selective LXR modulators that activate cholesterol efflux with minimal impact on SREBP-1c expression and triglyceride synthesis in liver would be expected to reduce atherosclerotic risk with an improved therapeutic index and minimize the potential for deleterious effects on metabolic balance.

The test procedures performed, and results obtained, are briefly described in the following sections:

I. Ligand-Binding Test Procedure for Human LXRβ

II. Ligand-Binding Test Procedure for Human LXRα

III. Quantitative Analysis of ABCA1 Gene Regulation in THP-1 Cells

IV. Results

### I. Ligand-Binding Test Procedure for Human LXRβ.

Ligand-binding to the human LXRβ was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

Tracer: ³H T0901317

Receptor source: E. coli extract from cells expressing biotinylated hLXRβ. Extract was made in a similar buffer as above, but with 50 mM TRIS.

### Day 1

Washed streptavidin and coated flash plates with wash buffer.

Diluted receptor extract to give Bmax ˜4000 cpm and add to the wells.

Wrapped the plates in aluminum foil and stored them at +4° C. over night.

### Day 2

Made a dilution series in DMSO of the test ligands.

Made a 5 nM solution of the radioactive tracer in buffer.

Mixed 250 μl diluted tracer with 50 of the test ligand from each concentration of the dilution series.

Washed the receptor-coated flash plates.

Added 200 μl per well of the ligand/radiolabel mixture to the receptor-coated flash plates.

Wrapped the plates in aluminum foil and incubate at +4° C. over night.

### Day 3

Aspirated wells, and wash the flashed plates. Sealed the plate.

Measured the remaining radioactivity in the plate.

### II. Ligand-Binding Test Procedure for Human LXRα.

Ligand-binding to the human LXRα was demonstrated for representative compounds of this invention by the following procedure.

Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

Tracer: ³H T0901317

Receptor source: E. coli extract from cells expressing biotinylated hLXRα. Extract was made in a similar buffer as above, but with 50 mM TRIS.

Day 1

Washed streptavidin and coated flash plates with wash buffer.

Diluted receptor extract to give Bmax ˜4000 cpm and add to the wells.

Wrapped the plates in aluminum foil and stored them at +4° C. over night.

Day 2

Made a dilution series in DMSO of the test ligands.

Made a 5 nM solution of the radioactive tracer in buffer.

Mixed 250 μl diluted tracer with 5 μl of the test ligand from each concentration of the dilution series.

Washed the receptor-coated flash plates.

Added 200 μl per well of the ligand/radiolabel mixture to the receptor-coated flash plates.

Wrapped the plates in aluminum foil and incubate at +4° C. over night.

Day 3

Aspirated wells, and wash the flashed plates. Sealed the plate.

Measured the remaining radioactivity in the plate.

### III. Quantitative Analysis of ABCA1 Gene Regulation in THP-1 Cells.

The compounds of formula (I) effect on the regulation of the ABCA1 gene was evaluated using the following procedure.

### Materials and Methods

Cell culture: The THP-1 monocytic cell line (ATCC # TIB-202) was obtained from American Type Culture Collection (Manassas, Va.) and cultured in RPMI 1640 medium (Gibco, Carlsbad, Calif.) containing 10% FBS, 2 mM L-glutamine, and 55 uM beta-Mercaptoethanol (BME). Cells were plated in 96-well format at a density of 7.5×10⁴ in complete medium containing 50-100 ng/ml phorbal 12,13-dibutyrate (Sigma, St. Louis, Mo.) for three days to induce differentiation into adherent macrophages. Differentiated THP-1 cells were treated with test compounds or ligands dissolved in DMSO (Sigma, D-8779) in culture medium lacking phorbal ester. Final concentrations of DMSO did not exceed 0.3% of the media volume. Dose response effects were measured in duplicate, in the range of 0.001 to 30 micromolar concentrations and treated cells were incubated for an additional 18 hrs prior to RNA isolation. Unstimulated cells treated with vehicle were included as negative controls on each plate. An LXR agonist reference, N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838), was dosed at 1.0 uM and served as a positive control. In antagonist mode, the compound under study is analyzed in the presence of 150 nM GW3965, trifluoromethyl-benzyl)-(2,2-diphenyl-ethyl)-amino]-propoxy]-phenyl)-acetic acid (Collins, J. L., J. Med. Chem. (2000), 45:1963-1966.). Results of antagonist analysis are expressed as antagonism and IC50 (in μM).

RNA isolation and quantitation: Total cellular RNA was isolated from treated cells cultured in 96-well plates using PrepStation 6100 (Applied Biosystems, Foster City, Calif.), according to the manufacturer's recommendations. RNA was resuspended in ribonuclease-free water and stored at −70° C. prior to analysis. RNA concentrations were quantitated with RiboGreen test procedure, #R-11490 (Molecular Probes, Eugene, Oreg.).

Gene expression analysis: Gene-specific mRNA quantitation was performed by real-time PCR with the Perkin Elmer Corp. chemistry on an ABI Prism 7700 Sequence detection system (Applied Biosystems, Foster City, Calif.) according to the manufacturer's instructions. Samples (50-100 ng) of total RNA were assayed in duplicate or triplicate in 50 ul reactions using one-step RT-PCR and the standard curve method to estimate specific mRNA concentrations. Sequences of gene-specific primer and probe sets were designed with Primer Express Software (Applied Biosystems, Foster City, Calif.). The human ABCA1 primer and probe sequences are: forward, CAACATGAATGCCATTTTCCAA, reverse, ATAATCCCCTGAACCCAAGGA, and probe, 6FAM-TAAAGCCATGCCCTCTGCAGGAACA-TAMRA. RT and PCR reactions were performed according to PE Applied Biosystem's protocol for Taqman Gold RT-PCR or Qiagen's protocol for Quantitect probe RT-PCR. Relative levels of ABCA1 mRNA are normalized using GAPDH mRNA or 18S rRNA probe/primer sets purchased commercially (Applied Biosystems, Foster City, Calif.).

### Statistics:

Mean, standard deviation and statistical significance of duplicate evaluations of RNA samples were assessed using ANOVA, one-way analysis of variance using SAS analysis.

### Reagents:

GAPDH Probe and Primers—Taqman GAPDH Control Reagents 402869 or 4310884E

18S Ribosomal RNA—Taqman 18S Control Reagents 4308329

10 Pack Taqman PCR Core Reagent Kit 402930

Qiagen Quantitect probe RT-PCR 204443.

### IV. Results

| | hLXRB binding assay | hLXRa binding assay |
|---|---|---|
| Example | Mean IC50 (uM) | Mean IC50 (uM) |
| 4 | 0.151 | 1.824 |
| 5 | 0.101 | 1.572 |
| 6 | 0.063 | 1.015 |
| 7 | 0.651 | 4.986 |
| 8 | >1 | >1 |
| 9 | 1 | >1 |
| 10 | 3.617 | 26.069 |
| 11 | 2.239 | >1 |
| 12 | >1 | >1 |
| 13 | 1.545 | >1 |
| 15 | 0.449 | 4.418 |
| 16 | >1 | >1 |
| 17 | >1 | >1 |
| 18 | 0.412 | 3.543 |
| 19 | 19.058 | 64.803 |
| 20 | 0.398 | 6.332 |
| 21 | 0.433 | 13.756 |
| 24 | 0.295 | 5.321 |
| 25 | 0.285 | 2.833 |
| 27 | 0.194 | 1.666 |
| 37 | 0.069 | 0.706 |
| 38 | 6.268 | |
| 40 | 0.097 | 1.136 |
| 41 | 0.175 | >1 |
| 42 | 0.452 | 3.251 |
| 43 | 0.309 | 20.337 |
| 44 | >1 | >1 |
| 45 | 5.289 | >1 |
| 49 | 0.005 | 0.177 |
| 50 | 0.005 | 0.236 |
| 51 | 0.002 | 0.119 |
| 52 | 0.014 | 0.284 |
| 53 | 0.008 | 0.233 |
| 54 | 0.005 | 0.176 |
| 55 | 0.035 | 0.782 |
| 56 | 0.044 | 0.787 |
| 57 | 0.214 | 2.341 |
| 58 | 0.02 | 1.356 |
| 59 | 0.019 | 2.371 |
| 60 | 0.016 | 0.524 |
| 61 | 0.049 | 1.791 |
| 62 | 0.063 | 1.432 |
| 63 | 0.181 | 3.382 |
| 64 | 0.081 | 1.826 |
| 65 | 0.152 | 2.954 |
| 66 | 0.099 | 1.535 |
| 67 | 0.036 | 0.712 |
| 68 | 0.015 | 0.597 |
| 69 | 0.095 | 0.265 |
| 73 | 0.182 | 2.517 |
| 75 | 0.02 | 0.718 |
| 76 | 0.153 | 0.714 |
| 77 | 0.344 | 10.388 |
| 78 | 0.026 | 0.325 |
| 93 | 0.047 | 0.714 |
| 94 | 0.069 | 1.045 |

Based on the results obtained in the standard pharmacological test procedures, the compounds of this invention can be useful in treating or inhibiting LXR mediated diseases. In particular, the compounds of this invention can be useful in the treatment and inhibition of atherosclerosis and atherosclerotic lesions, lowering LDL cholesterol levels, increasing HDL cholesterol levels, increasing reverse cholesterol transport, inhibiting cholesterol absorption, treatment or inhibition of cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A number of embodiments of the invention have been described. Nevertheless, it will be understood that various modifications may be made without departing from the spirit and scope of the invention. Accordingly, other embodiments are in the claims.

What is claimed is:

1. A compound having formula (I): 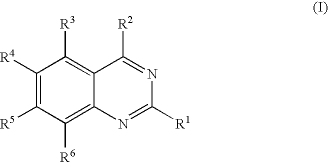
wherein:
R¹ is:
(i) hydrogen; or
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{b}; or
(iv) C₃-C₂₀ cycloalkyl, C₃-C₂₀ cycloalkenyl, heterocyclyl including 3-20 atoms, heterocycloalkenyl including 3-20 atoms, C₇-C₂₀ aralkyl, or heteroaralkyl including 6-20 atoms, each of which is optionally substituted with from 1-10 R^{c}; or
(v) C₆-C₁₈ aryl or heteroaryl including 5-16 atoms, each of which is optionally substituted with from 1-10 R^{d};
R² is C₆-C₁₈ aryl or heteroaryl including 5-16 atoms, each of which is:
(i) substituted with from 1-5 R⁷, and
(ii) optionally substituted with from 1-4 R^{e}; wherein:
R⁷ is WA, wherein:
W at each occurrence is, independently, a bond; —O—; —NR⁸— wherein R⁸ is hydrogen, C₁-C₆ alkyl, C₃-C₇ cycloalkyl, or C₆-C₁₀ aryl or heteroaryl including 5-10 atoms in which the aryl or heteroaryl group is optionally substituted with from 1-5 R^{d}; C₁₋₆ alkylene, C₂₋₆ alkenylene, or C₂₋₆ alkynylene, each of which is optionally substituted with from 1-5 R^{f}; —W¹(C₁₋₆ alkylene)-; or —(C₁₋₆ alkylene)W¹—;
W¹ at each occurrence is, independently, —O— or —NR⁸—; and
A at each occurrence is, independently, C₆-C₁₈ aryl or heteroaryl including 5-16 atoms, each of which is:
(i) substituted with from 1-5 R⁹, and
(ii) optionally further substituted with from 1-10 R^{g};
R⁹ at each occurrence is, independently:
(i) —W²—S(O)ₙR¹⁰ or —W²—S(O)ₙNR¹¹R¹²; or
(ii) —W²—C(O)OR¹³; or
(iii) —W²—C(O)NR¹¹R¹²; or
(iv) —W²—CN; or
(v) C₁-C₁₂ alkyl or C₁-C₁₂ haloalkyl, each of which is:
(a) substituted with from 1-3 R^{h}, and
(b) optionally further substituted with from 1-5 R^{a};
or
(vi) C₇-C₂₀ aralkyl or heteroaralkyl including 6-20 atoms, each of which is:
(a) substituted with from 1-3 R^{h}, and
(b) optionally further substituted with from 1-5 substituents independently selected from R^{a}; C₁-C₆ alkyl, which is optionally substituted with from 1-3 R^{a}; C₁-C₆ haloalkyl; C₆-C₁₀ aryl, which is optionally substituted with from 1-10 R^{d}; halo; C₂-C₆ alkenyl; or C₂-C₆ alkynyl;
or
(vii) —NR¹⁴R¹⁵;
wherein:
W² at each occurrence is, independently, a bond; C₁₋₆ alkylene optionally substituted with from 1-3 R^{f}; C₂₋₆ alkenylene; C₂₋₆ alkynylene; C₃₋₆ cycloalkylene;
—O(C₁₋₆ alkylene)-, or —NR⁸(C₁₋₆ alkylene)-;
n at each occurrence is, independently, 1 or 2;
R¹⁰ at each occurrence is, independently:
(i) C₁-C₂₀ alkyl or C₁-C₂₀ haloalkyl, each of which is optionally substituted with from 1-10 R^{a}; or
(ii) C₂-C₂₀ alkenyl or C₂-C₂₀ alkynyl, each of which is optionally substituted with from 1-10 R^{b}; or
(iii) C₃-C₂₀ cycloalkyl, C₃-C₂₀ cycloalkenyl, C₇-C₂₀ aralkyl, or heteroaralkyl including 6-20 atoms, each of which is optionally substituted with from 1-10 R^{c}; or
(iv) C₆-C₁₈ aryl or heteroaryl including 5-16 atoms, each of which is optionally substituted with from 1-10 R^{d};
R¹¹ and R¹² are each, independently, hydrogen; R¹⁰; or heterocyclyl including 3-20 atoms or a heterocycloalkenyl including 3-20 atoms, each of which is optionally substituted with from 1-5 R^{c}; or
R¹¹ and R¹² together with the nitrogen atom to which they are attached form a heterocyclyl including 3-20 atoms or a heterocycloalkenyl including 3-20 atoms, each of which is optionally substituted with from 1-5 R^{c};
R¹³ at each occurrence is, independently, hydrogen or R¹⁰;
at each occurrence of —NR¹⁴R¹⁵, one of R¹⁴ and R¹⁵ is hydrogen or C₁-C₃ alkyl; and the other of R¹⁴ and R¹⁵ is:
(i) —S(O)ₙR¹⁰; or
(ii) —C(O)OR¹³; or
(iii) —C(O)NR¹¹R¹²; or
(iv) —CN; or
(v) C₁-C₁₂ alkyl or C₁-C₁₂ haloalkyl, each of which is:
(a) substituted with from 1-3 R^{h}, and
(b) optionally further substituted with from 1-5 R^{a};
or
(vi) C₇-C₂₀ aralkyl or heteroaralkyl including 6-20 atoms, each of which is:
(a) substituted with from 1-3 R^{h}, and
(b) optionally further substituted with from 1-5 substituents independently selected from R^{a}; C₁-C₆ alkyl, which is optionally substituted with from 1-3 R^{a}; C₁-C₆ haloalkyl; C₆-C₁₀ aryl, which is optionally substituted with from 1-10 R^{d}; halo; C₂-C₆ alkenyl; or C₂-C₆ alkynyl;
each of R³, R⁴, and R⁵ is, independently:
(i) hydrogen; or
(ii) halo; or
(iii) C₁-C₆ alkyl or C₁-C₆ haloalkyl, each of which is optionally substituted with from 1-3 R^{a}; or
(iv) NRⁱR^{j}, wherein each of Rⁱ and R^{j} is, independently, hydrogen or C₁-C₃ alkyl; nitro; azido; hydroxy; C₁-C₆ alkoxy; C₁-C₆ haloalkoxy; C₆-C₁₀ aryloxy; heteroaryloxy including 5-10 atoms, each of which is optionally substituted with from 1-5 R^{d}; C₇-C₁₀ aralkoxy, heteroaralkoxy including 6-10 atoms, C₃-C₆ cycloalkoxy, C₃-C₆ cycloalkenyloxy, heterocyclyloxy including 3-6 atoms, or heterocycloalkenyloxy including 3-6 atoms, each of which is optionally substituted with from 1-5 R^{c}; mercapto; C₁-C₆ thioalkoxy; C₁-C₆ thiohaloalkoxy; C₆-C₁₀ thioaryloxy or thioheteroaryloxy including 5-10 atoms, each of which is optionally substituted with from 1-10 R^{d}; C₇-C₁₀ thioaralkoxy, thioheteroaralkoxy including 6-10 atoms, C₃-C₆ thiocycloalkoxy, C₃-C₆ thiocycloalkenyloxy, thioheterocyclyloxy including 3-6 atoms, or thioheterocycloalkenyloxy including 3-6 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or
(v) C₂-C₆ alkenyl or C₂-C₆ alkynyl, each of which is optionally substituted with from 1-10 R^{b}; or
(vi) C₇-C₁₀ aralkyl, heteroaralkyl including 6-10 atoms, C₃-C₆ cycloalkyl, C₃-C₆ cycloalkenyl, heterocyclyl including 3-6 atoms, or heterocycloalkenyl including 3-6 atoms, each of which is optionally substituted with from 1-3 R^{c}; or
(vii) C₆-C₁₀ aryl or heteroaryl including 5-10 atoms, each of which is optionally substituted with from 1-10 R^{d};
R⁶ is:
(i) halo; or
(ii) C₁-C₆ alkyl or C₁-C₆ haloalkyl, each of which is optionally substituted with from 1-3 R^{a}; or
(iii) nitro; azido; C₁-C₆ alkoxy; C₁-C₆haloalkoxy; C₆-C₁₀ aryloxy or heteroaryloxy including 5-10 atoms, each of which is optionally substituted with from 1-5 R^{d}; C₇-C₁₀ aralkoxy, heteroaralkoxy including 6-10 atoms, C₃-C₆ cycloalkoxy, C₃-C₆ cycloalkenyloxy, heterocyclyloxy including 3-6 atoms, or heterocycloalkenyloxy including 3-6 atoms, each of which is optionally substituted with from 1-5 R^{c}; C₁-C₆ thioalkoxy; C₁-C₆ thiohaloalkoxy; C₆-C₁₀ thioaryloxy or thioheteroaryloxy including 5-10 atoms, each of which is optionally substituted with from 1-10 R^{d}; C₇-C₁₀ thioaralkoxy, thioheteroaralkoxy including 6-10 atoms, C₃-C₆ thiocycloalkoxy, C₃-C₆ thiocycloalkenyloxy, thioheterocyclyloxy including 3-6 atoms, or thioheterocycloalkenyloxy including 3-6 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or
(iv) C₂-C₆ alkenyl or C₂-C₆ alkynyl, each of which is optionally substituted with from 1-10 R^{b}; or
(v) C₇-C₁₀ aralkyl, heteroaralkyl including 6-10 atoms, C₃-C₆ cycloalkyl, C₃-C₆ cycloalkenyl, heterocyclyl including 3-6 atoms, or heterocycloalkenyl including 3-6 atoms, each of which is optionally substituted with from 1-3 R^{c}; or
(vi) C₆-C₁₀ aryl or heteroaryl including 5-10 atoms, each of which is optionally substituted with from 1-10 R^{d};
R^{a} at each occurrence is, independently:
(i) NR^{m}Rⁿ; nitro; azido; hydroxy; oxo; thioxo; ═NR^{p}; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or
(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{c};
R^{a′} at each occurrence is, independently, NR^{m}Rⁿ; nitro; azido; hydroxy; oxo; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; C₃-C₂₀ cycloalkyl, C₃-C₂₀ cycloalkenyl, heterocyclyl including 3-20 atoms, or heterocycloalkenyl including 3-20 atoms;
R^{b} at each occurrence is, independently:
(i) halo NR^{m}Rⁿ; nitro; azido; hydroxy; oxo; thioxo; ═NR^{p}; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or
(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{c}; or
(iii) C₆-C₁₈ aryl or heteroaryl including 5-16 atoms, each of which is optionally substituted with from 1-10 R^{d};
R^{c} at each occurrence is, independently:
(i) halo; NR^{m}Rⁿ; nitro; azido; hydroxy; oxo; thioxo; ═NR^{p}; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{b}; or
(iv) C₆-C₁₈ aryl or heteroaryl including 5-16 atoms, each of which is optionally substituted with from 1-10 R^{d}; or
(v) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{c′};
R^{c′} at each occurrence is, independently, R^{e′}; halo; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₁-C₂₀ alkyl or C₁-C₂₀ haloalkyl, each of which is optionally substituted with from 1-10 R^{a}; C₂-C₂₀ alkenyl; C₂-C₂₀ alkynyl; or C₆-C₁₈ aryl or heteroaryl including 5-16 atoms, each of which is optionally substituted with from 1-10 R^{d};
R^{d} at each occurrence is, independently:
(i) halo; NR^{m}Rⁿ; nitro; azido; hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d′}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d′}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ); or
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{b}; or
(iv) C₇-C₂₀ aralkyl, heteroaralkyl including 6-20 atoms, C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{c}; or
(v) C₆-C₁₈ aryl or heteroaryl including 5-16 atoms, each of which is optionally substituted with from 1-10 R^{d′};
R^{d′} at each occurrence is, independently, halo; NR^{m}Rⁿ; nitro; azido; hydroxy; C₁-C₂₀ alkyl, C₁-C₂₀ haloalkyl, C₂-C₂₀ alkenyl; C₂-C₂₀ alkynyl; C₃-C₂₀ cycloalkyl; C₃-C₂₀ cycloalkenyl, heterocyclyl including 3-20 atoms; heterocycloalkenyl including 3-20 atoms; C₇-C₂₀ aralkyl; heteroaralkyl including 6-20 atoms; C₁-C₂₀ alkoxy; C₁-C₂₀ haloalkoxy; C₆-C₁₈ aryloxy; heteroaryloxy; C₇-C₂₀ aralkoxy; heteroaralkoxy including 6-20 atoms; C₃-C₁₆ cycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₇-C₂₀ thioaralkoxy, thioheteroaralkoxy including 6-20 atoms, C₃-C₁₆ thiocycloalkoxy C₃-C₂₀ thiocycloalkenyloxy, thioheterocyclyloxy including 3-20 atoms, or thioheterocycloalkenyloxy including 3-20 atoms; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ);
each of R^{e} at each occurrence is, independently, C₁-C₆ alkyl, optionally substituted with from 1-3 R^{a}; C₁-C₆ haloalkyl; mercapto; C₁-C₆ thioalkoxy optionally substituted with from 1-3 R^{a}; C₆-C₁₀ aryl or C₆-C₁₀ aryloxy, each of which is optionally substituted with from 1-10 R^{d}; halo; hydroxyl; NR^{m}Rⁿ; nitro; C₂-C₆ alkenyl; C₂-C₆ alkynyl; C₁-C₆ alkoxy; C₁-C₆ haloalkoxy; cyano; —C(O)OR^{k}; or —C(O)R^{k};
R^{f} at each occurrence is, independently, mercapto; C₁-C₆ thioalkoxy optionally substituted with from 1-3 R^{e}; C₆-C₁₀ aryl or C₆-C₁₀ aryloxy, each of which is optionally substituted with from 1-10 R^{h}; halo; hydroxyl; NR^{m}Rⁿ; nitro; C₂-C₆ alkenyl; C₂-C₆ alkynyl; C₁-C₆ alkoxy; C₁-C₆ haloalkoxy; cyano; —C(O)OR^{k}; or —C(O)R^{k};
R^{g} at each occurrence is, independently:
(i) halo; NR^{m}Rⁿ; nitro; azido; hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{c}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{c}; cyano; —C(O)R^{k}, —C(O)OR^{k}; —OC(O)R^{k}; —C(O)SR^{k}; —SC(O)R^{k}; —C(S)SR^{k}; —SC(S)R^{k}; —C(O)NR^{m}Rⁿ; —NR^{o}C(O)R^{k}; —C(NR^{p})R^{k}; —OC(O)NR^{m}Rⁿ; —NR^{o}C(O)NR^{m}Rⁿ; —NR^{o}C(O)OR^{k}; —S(O)ₙR^{q}, wherein n is 1 or 2; —NR^{o}S(O)ₙR^{q}; or —P(O)(OR^{m})(ORⁿ);
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{h};
R^{h} at each occurrence is, independently, hydroxyl, C₁-C₁₂ alkoxy, or C₁-C₁₂ haloalkoxy; C₃-C₁₀ cycloalkoxy or C₃-C₁₀ cycloalkenyloxy, each of which is optionally substituted with from 1-5 R^{c}; or C₆-C₁₀ aryloxy or heteroaryloxy including 5-10 atoms, each of which is optionally substituted with from 1-5 R^{d};
each of R^{m}, Rⁿ, R^{o}, and R^{p}, at each occurrence is, independently:
(i) hydrogen; or
(ii) R¹⁰; or
(iii) heterocyclyl including 3-20 atoms or a heterocycloalkenyl including 3-20 atoms, each of which is optionally substituted with from 1-5 R^{c}; or
(iv) —C(O)R^{k}, —C(O)OR^{k}; or —S(O)ₙR^{q};
R^{k} at each occurrence is, independently:
(i) hydrogen; or
(ii) R¹⁰; or
(iii) heterocyclyl including 3-20 atoms or a heterocycloalkenyl including 3-20 atoms, each of which is optionally substituted with from 1-5 R^{c}; and
R^{q} at each occurrence is, independently, R^{k}, OR^{k}, or NR^{m}Rⁿ;
or an N-oxide and/or a pharmaceutically acceptable salt thereof.

2. The compound of claim 1, wherein R¹ is hydrogen.

3. The compound of claim 1, wherein R¹ is C₁-C₃ alkyl or C₁-C₃ haloalkyl.

4. The compound of claim 3, wherein R¹ is CH₃.

5. The compound of claim 3, wherein R¹ is CF₃.

6. The compound of claim 1, wherein R¹ is C₆-C₁₀ aryl or heteroaryl including 5-10 atoms, each of which is optionally substituted with from 1-5 R^{d}.

7. The compound of claim 6, wherein R¹ is phenyl, which is optionally substituted with from 1-5 R^{d}.

8. The compound of claim 6, wherein R¹ is thienyl, which is optionally substituted with from 1-5 R^{d}.

9. The compound of claim 1, wherein R¹ is C₃-C₈ cycloalkyl or heterocyclyl including 3-8 atoms, each of which is optionally substituted with from 1-3 R^{c}.

10. The compound of claim 1, wherein R² is C₆-C₁₀ aryl, which is (a) substituted with from 1-2 R⁷; and (b) optionally substituted with from 1-2 R^{e}.

11. The compound of claim 1, wherein R² is phenyl, which is (a) substituted with 1 R⁷; and (b) optionally substituted with 1 R^{e}.

12. The compound of claim 1, wherein R² is phenyl, which is substituted with 1 R⁷.

13. The compound of claim 1, wherein R² has formula (A-2): 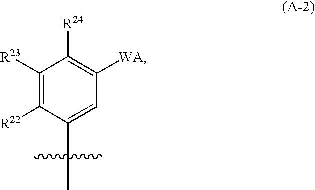
each of R²², R²³, and R²⁴ is, independently, hydrogen or R^{e}.

14. The compound of claim 13, wherein each of R²², R²³, and R²⁴ is hydrogen.

15. The compound of claim 13, wherein one of R²², R²³, and R²⁴ is R^{e}, and the other two are hydrogen.

16. The compound of claim 13, wherein R²² is R^{e}, and each of R²³ and R²⁴ is hydrogen.

17. The compound of claim 16, wherein R²² is halo.

18. The compound of claim 17, wherein R²² is chloro.

19. The compound of claim 1, wherein W is —O—.

20. The compound of claim 1, wherein W is a bond.

21. The compound of claim 1, wherein W is —W¹(C₁₋₆ alkylene)-.

22. The compound of claim 21, wherein W¹ is —O—.

23. The compound of claim 1, wherein A is C₆-C₁₀ aryl, which is (a) substituted with from 1-2 R⁹; and (b) optionally substituted with from 1-4 R^{g}.

24. The compound of claim 1, wherein A is C₆-C₁₀ aryl, which is (a) substituted with 1 R⁹; and (b) optionally substituted with from 1-4 R^{g}.

25. The compound of claim 1, wherein A is phenyl, which is (a) substituted with 1 R⁹; and (b) optionally substituted with from 1-4 R^{g}.

26. The compound of claim 1, wherein A has formula (B-1): 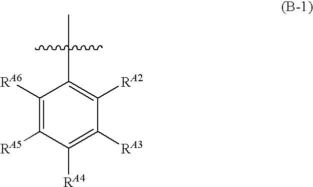
wherein:
one of R^{A3} and R^{A4} is R⁹, the other of R^{A3} and R^{A4} is hydrogen; and
each of R^{A2}, R^{A5}, and R^{A6} is, independently, hydrogen or R^{g}.

27. The compound of claim 1, wherein R⁹ is —W²—S(O)ₙR¹⁰.

28. The compound of claim 1, wherein R⁹ is —W²—C(O)OR¹³.

29. The compound of claim 1, wherein R² has formula (C-1): 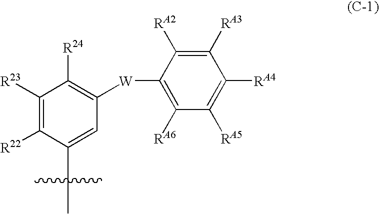
wherein:
each of R²², R²³, and R²⁴ is, independently, hydrogen or R^{e}; and
one of R^{A2}, R^{A3}, R^{A4}, R^{A5}, and R^{A6} is R⁹, and the others are each, independently, hydrogen or R^{g}.

30. The compound of claim 29, wherein each of R²², R²³, and R²⁴ is hydrogen.

31. The compound of claim 29, wherein one of R²², R²³, and R²⁴ is R^{e}, and the other two are hydrogen.

32. The compound of claim 29, wherein R²² is R^{e}, and each of R²³ and R²⁴ is hydrogen.

33. The compound of claim 32, wherein R²² is halo.

34. The compound of claim 33, wherein R²² is chloro.

35. The compound of claim 29, wherein W is —O—.

36. The compound of claim 29, wherein W is a bond.

37. The compound of claim 29, wherein W is —OCH₂—.

38. The compound of claim 29, wherein one of R^{A3} and R^{A4} is R⁹, and the other of R^{A3} and R^{A4} is hydrogen; and each of R^{A2}, R^{A5}, and R^{A6} is, independently, hydrogen or R^{g}.

39. The compound of claim 38, wherein R^{A3} is —W²—S(O)ₙR¹⁰.

40. The compound of claim 39, wherein each of R^{A2}, R^{A5}, and R^{A6} is hydrogen.

41. The compound of claim 39, wherein W² is a bond.

42. The compound of claim 39, wherein n is 2.

43. The compound of claim 39, wherein R¹⁰ is C₁-C₁₀ alkyl, optionally substituted with from 1-2 R^{a}.

44. The compound of claim 43, wherein R¹⁰ is C₁-C₃ alkyl.

45. The compound of claim 44, wherein R¹⁰ is CH₃.

46. The compound of 43, wherein R¹⁰ is C₂-C₈ alkyl substituted with 1 R^{a}.

47. The compound of claim 46, wherein R^{a} is hydroxyl or C₁-C₃ alkoxy.

48. The compound of claim 39, wherein R^{A5} is hydrogen or R^{g}, and each of R^{A2} and R^{A6} is hydrogen.

49. The compound of claim 38, wherein R^{A4} is —W²—C(O)OR¹³.

50. The compound of claim 49, wherein R¹³ is hydrogen.

51. The compound of claim 49, wherein R¹³ is C₁-C₃ alkyl.

52. The compound of claim 49, wherein W² is C₁-C₃ alkylene.

53. The compound of claim 49, wherein W² is CH₂.

54. The compound of claim 49, wherein W² is a bond.

55. The compound of claim 49, wherein each of R^{A2}, R^{A5}, and R^{A6} is hydrogen.

56. The compound of claim 1, wherein each of R³, R⁴, and R⁵ is, independently:
(i) hydrogen; or
(ii) halo; or
(iii) C₁-C₆ alkyl or C₁-C₆ haloalkyl, each of which is optionally substituted with from 1-3 R^{e}; or
(iv) C₃-C₆ cycloalkyl, which is optionally substituted with from 1-3 R^{c}; or
(v) C₆-C₁₀ aryl, which is optionally substituted with from 1-10 R^{d}.

57. The compound of claim 1, wherein each of R³, R⁴, and R⁵ is hydrogen.

58. The compound of claim 1, wherein R⁶ is:
(i) halo; or
(ii) C₁-C₆ alkyl or C₁-C₆ haloalkyl, each of which is optionally substituted with from 1-3 R^{e};
(iii) cyano; —C(O)NR^{m}Rⁿ; or —S(O)ₙR^{q}, wherein n is 1 or 2.

59. The compound of claim 1, wherein R⁶ is C₁-C₆ haloalkyl.

60. The compound of claim 59, wherein R⁶ is C₁-C₃ perfluoroalkyl.

61. The compound of claim 60, wherein R⁶ is CF₃.

62. The compound of claim 1, wherein R⁶ is halo.

63. The compound of claim 62, wherein R⁶ is chloro.

64. The compound of claim 1, wherein the compound has formula (VI): 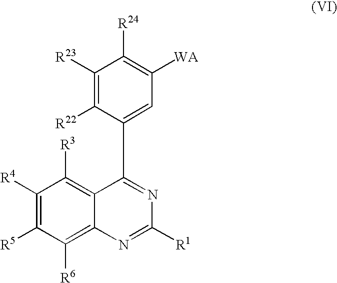
wherein:
R¹ is:
(i) hydrogen; or
(ii) C₁-C₃ alkyl or C₁-C₃ haloalkyl; or
(iii) phenyl or heteroaryl including 5-6 atoms, each of which is optionally substituted with from 1-5 R^{d}; or
(iv) C₃-C₈ cycloalkyl or heterocyclyl including 3-8 atoms, each of which is optionally substituted with from 1-3 R^{c};
each of R³, R⁴, and R⁵ is, independently:
(i) hydrogen; or
(ii) halo; or
(iii) C₁-C₃ alkyl or C₁-C₃ haloalkyl, each of which is optionally substituted with from 1-3 R^{a}; or
(iv) C₃-C₆ cycloalkyl, which is optionally substituted with from 1-3 R^{c}; or
(v) C₆-C₁₀ aryl, which is optionally substituted with from 1-10 R^{d};
R⁶ is:
(i) halo; or
(ii) C₁-C₃ alkyl or C₁-C₃ haloalkyl, each of which is optionally substituted with from 1-3 R^{e}; or
(iii) cyano; —C(O)NR^{m}Rⁿ; or —S(O)ₙR^{q}, wherein n is 1 or 2; and
each of R²², R²³, and R²⁴ is, independently, hydrogen or R^{e}.

65. The compound of claim 64, wherein R¹ is hydrogen.

66. The compound of claim 64, wherein R¹ is CH₃ or CF₃.

67. The compound of claim 64, wherein R¹ is phenyl or thienyl, each of which is optionally substituted with from 1-5 R^{d}.

68. The compound of claim 64, wherein W is —O—.

69. The compound of claim 64, wherein W is a bond.

70. The compound of claim 64, wherein W is —OCH₂—.

71. The compound of claim 64, wherein A has formula (B-1), wherein one of R^{A3} and R^{A4} is R⁹, and the other of R^{A3} and R^{A4} is hydrogen; and each of R^{A2}, R^{A5}, and R^{A6} is, independently, hydrogen or R^{g}.

72. The compound of claim 71, wherein R^{A3} is —W²—S(O)ₙR¹⁰, wherein W² is a bond, and n is 2.

73. The compound of claim 72, wherein R¹⁰ is C₁-C₁₀ alkyl, optionally substituted with from 1-2 R^{a}.

74. The compound of claim 73, wherein R¹⁰ is CH₃, CH₂CH₃, or isopropyl.

75. The compound of 73, wherein R¹⁰ is C₂-C₈ alkyl substituted with 1 R^{a}.

76. The compound of claim 75, wherein R^{a} is hydroxyl or C₁-C₃ alkoxy.

77. The compound of claim 72, wherein R^{A5} is hydrogen or R^{c}, and each of R^{A2} and R^{A6} is hydrogen.

78. The compound of claim 71, wherein R^{A4} is —W²—C(O)OR¹³.

79. The compound of claim 78, wherein R¹³ is hydrogen or C₁-C₃ alkyl.

80. The compound of claim 78, wherein W² is CH₂.

81. The compound of claim 78, wherein each of R^{A2}, R^{A5}, and R^{A6} is hydrogen.

82. The compound of claim 64, wherein each of R³, R⁴, and R⁵ is hydrogen.

83. The compound of claim 64, wherein each of R²², R²³, and R²⁴ is hydrogen.

84. The compound of claim 64, wherein one of R²², R²³, and R²⁴ is R^{e}, and the other two are hydrogen.

85. The compound of claim 64, wherein R²² is R^{e}, and each of R²³ and R²⁴ is hydrogen.

86. The compound of claim 85, wherein R²² is chloro.

86. The compound of claim 64, wherein R⁶ is CF₃.

87. The compound of claim 64, wherein R⁶ is chloro.

88. The compound of claim 1, wherein the compound is selected from the group consisting of:
4-(3-methoxyphenyl)-8-(trifluoromethyl)quinazoline;
3-(8-(Trifluoromethyl)quinazolin-4-yl)phenol;
3-[2-methyl-8-(trifluoromethyl)quinazolin-4-yl]phenol;
4-{3-[3-(Methylsulfonyl)phenoxy]phenyl}-8-(trifluoromethyl)quinazoline;
4-{3-[3-(ethylsulfonyl)phenoxy]phenyl}-8-(trifluoromethyl)quinazoline;
4-{3-[3-(isopropylsulfonyl)phenoxy]phenyl}-8-(trifluoromethyl)quinazoline;
4-(3-{3-[(3-methoxypropyl)sulfonyl]phenoxy}phenyl)-8-(trifluoromethyl)quinazoline;
4-{3-[3-chloro-5-(propyl sulfonyl)phenoxy]phenyl}-8-(trifluoromethyl)quinazoline;
3-[(3-chloro-5-{3-[8-(trifluoromethyl)quinazolin-4-yl]phenoxy}phenyl)sulfonyl]propan-1-ol;
4-{3-[4-(methylsulfonyl)phenoxy]phenyl}-8-(trifluoromethyl)quinazoline;
4-{3-[4-(ethylsulfonyl)phenoxy]phenyl}-8-(trifluoromethyl)quinazoline;
4-{3-[4-(propylsulfonyl)phenoxy]phenyl}-8-(trifluoromethyl)quinazoline;
4-{3-[4-(isopropylsulfonyl)phenoxy]phenyl}-8-(trifluoromethyl)quinazoline;
4-{3-[2-(methylsulfonyl)phenoxy]phenyl}-8-(trifluoromethyl)quinazoline;
4-{3-[3-(propylsulfonyl)phenoxy]phenyl}-8-(trifluoromethyl)quinazoline;
4-{3-[3-(isobutylsulfonyl)phenoxy]phenyl}-8-(trifluoromethyl)quinazoline;
4-(3-{3-[(3-methylbutyl)sulfonyl]phenoxy}phenyl)-8-(trifluoromethyl)quinazoline;
3-[(3-{3-[8-(trifluoromethyl)quinazolin-4-yl]phenoxy}phenyl)sulfonyl]propan-1-ol;
3-[(4-{3-[8-(trifluoromethyl)quinazolin-4-yl]phenoxy}phenyl)sulfonyl]propan-1-ol;
4-{3-[3-fluoro-5-(methylsulfonyl)phenoxy]phenyl}-8-(trifluoromethyl)quinazoline;
2-methyl-4-{3-[3-(methylsulfonyl)phenoxy]phenyl}-8-(trifluoromethyl)quinazoline;
8-chloro-4-{3-[3-(methyl sulfonyl)phenoxy]phenyl}quinazoline;
4-(3-{[3-(methylsulfonyl)benzyl]oxy}phenyl)-8-(trifluoromethyl)quinazoline;
4-(3-Bromo-phenyl)-8-trifluoromethyl-quinazoline;
4-(3′-Methanesulfonyl-biphenyl-3-yl)-8-trifluoromethyl-quinazoline;
4-(4′-Methanesulfonyl-biphenyl-3-yl)-8-trifluoromethyl-quinazoline;
4-(3-bromophenyl)-2-phenyl-8-(trifluoromethyl)quinazoline;
4-(3-bromophenyl)-2-(thiophen-2-yl)-8-(trifluoromethyl)quinazoline;
4-(3-Bromo-phenyl)-2-methyl-8-trifluoromethyl-quinazoline;
4-(3-bromophenyl)-2,8-bis(trifluoromethyl)quinazoline;
4-(3-bromophenyl)-2-ethyl-8-(trifluoromethyl)quinazoline;
4-(3-bromophenyl)-2-propyl-8-(trifluoromethyl)quinazoline;
4-(3-bromophenyl)-2-isopropyl-8-(trifluoromethyl)quinazoline;
4-(3-methoxyphenyl)-2-methyl-8-(trifluoromethyl)quinazoline;
4-(3′-Methanesulfonyl-biphenyl-3-yl)-2-methyl-8-trifluoromethyl-quinazoline;
4-(4′-Methanesulfonyl-biphenyl-3-yl)-2-methyl-8-trifluoromethyl-quinazoline;
4-[3′-(methylsulfonyl)biphenyl-3-yl]-2,8-bis(trifluoromethyl)quinazoline;
4-[4-chloro-3′-(methylsulfonyl)biphenyl-3-yl]-8-(trifluoromethyl)quinazoline;
2-ethyl-4-[3′-(methylsulfonyl)biphenyl-3-yl]-8-(trifluoromethyl)quinazoline;
4-[3′-(methylsulfonyl)biphenyl-3-yl]-2-propyl-8-(trifluoromethyl)quinazoline;
2-isopropyl-4-[3′-(methylsulfonyl)biphenyl-3-yl]-8-(trifluoromethyl)quinazoline;
4-[3′-(methylsulfonyl)biphenyl-3-yl]-2-phenyl-8-(trifluoromethyl)quinazoline;
2-methyl-2-[4-({3-[8-(trifluoromethyl)quinazolin-4-yl]phenoxy}methyl)phenyl]propanoic acid;
[4-({3-[8-(trifluoromethyl)quinazolin-4-yl]phenoxy}methyl)phenyl]acetic acid;
4-chloro-8-(trifluoromethyl)quinazoline;
4-(2-chloro-5-methoxyphenyl)-8-(trifluoromethyl)quinazoline;
4-chloro-3-[8-(trifluoromethyl)quinazolin-4-yl]phenol;
4-{2-chloro-5-[3-(methylsulfonyl)phenoxy]phenyl}-8-(trifluoromethyl)quinazoline;
4-{2-chloro-5-[3-(ethylsulfonyl)phenoxy]phenyl}-8-(trifluoromethyl)quinazoline;
4-{2-chloro-5-[3-(isopropylsulfonyl)phenoxy]phenyl}-8-(trifluoromethyl)quinazoline;
4-{2-fluoro-5-[3-(methylsulfonyl)phenoxy]phenyl}-8-(trifluoromethyl)quinazoline;
4-{5-[3-(ethylsulfonyl)phenoxy]-2-fluorophenyl}-8-(trifluoromethyl)quinazoline;
4-{2-fluoro-5-[3-(isopropylsulfonyl)phenoxy]phenyl}-8-(trifluoromethyl)quinazoline;
4-{2-fluoro-5-[3-fluoro-5-(methylsulfonyl)phenoxy]phenyl}-8-(trifluoromethyl)quinazoline;
4-(2-fluoro-5-{[3-(methylsulfonyl)benzyl]oxy}phenyl)-8-(trifluoromethyl)quinazoline;
4-{5-[3-chloro-5-(methylsulfonyl)phenoxy]-2-fluorophenyl}-8-(trifluoromethyl)quinazoline;
4-{2-chloro-5-[3-(methylsulfonyl)phenoxy]phenyl}-2-methyl-8-(trifluoromethyl)-quinazoline;
4-{2-chloro-5-[3-(ethylsulfonyl)phenoxy]phenyl}-2-methyl-8-(trifluoromethyl)quinazoline;
4-{2-chloro-5-[3-(isopropylsulfonyl)phenoxy]phenyl}-2-methyl-8-(trifluoromethyl)-quinazoline;
4-{2-chloro-5-[3-fluoro-5-(methylsulfonyl)phenoxy]phenyl}-2-methyl-8-(trifluoromethyl)-quinazoline;
4-{2-chloro-5-[3-(ethylsulfonyl)-5-fluorophenoxy]phenyl}-2-methyl-8-(trifluoromethyl)-quinazoline;
4-(2-chloro-5-{[3-(methylsulfonyl)benzyl]oxy}phenyl)-2-methyl-8-(trifluoromethyl)quinazoline;
8-chloro-4-{2-fluoro-5-[3-fluoro-5-(methylsulfonyl)phenoxy]phenyl}quinazoline;
8-chloro-4-{5-[3-(ethylsulfonyl)-5-fluorophenoxy]-2-fluorophenyl}quinazoline;
8-chloro-4-(2-fluoro-5-{[3-(methylsulfonyl)benzyl]oxy}phenyl)quinazoline;
8-chloro-4-{2-fluoro-5-[3-(ethylsulfonyl)phenoxy]phenyl}quinazoline;
8-chloro-4-(2-fluoro-5-{3-[(1-methylethyl)sulfonyl]phenoxy}phenyl)quinazoline;
8-chloro-4-[2-fluoro-5-(3-{[3-(tetrahydro-2H-pyran-2-yloxy)propyl]sulfonyl}phenoxy)-phenyl]quinazoline;
3-[3-(8-chloroquinazolin-4-yl)-4-fluorophenoxy]benzonitrile;
3-[3-(8-chloroquinazolin-4-yl)-4-fluorophenoxy]benzoic acid;
3-[(3-{4-chloro-3-[2-methyl-8-(trifluoromethyl)quinazolin-4-yl]phenoxy}phenyl)sulfonyl]-propan-1-ol;
3-({3-[3-(8-chloroquinazolin-4-yl)-4-fluorophenoxy]phenyl}sulfonyl)propan-1-ol;
Methyl 3-[3-(8-chloroquinazolin-4-yl)-4-fluorophenoxy]benzoate;
8-chloro-4-{2-fluoro-5-[3-(methylsulfonyl)phenoxy]phenyl}quinazoline;
2-cyclopropyl-4-[3′-(methylsulfonyl)biphenyl-3-yl]-8-(trifluoromethyl)quinazoline;
4-{2-chloro-5-[3-(methylsulfonyl)phenoxy]phenyl}-8-methoxyquinazoline;
8-chloro-4-{2-chloro-5-[3-(methylsulfonyl)phenoxy]phenyl}quinazoline;
3-({3-[4-chloro-3-(8-chloroquinazolin-4-yl)phenoxy]phenyl}sulfonyl)propan-1-ol;
8-chloro-4-{2-chloro-5-[3-(ethylsulfonyl)phenoxy]phenyl}quinazoline;
8-chloro-4-{2-chloro-5-[3-(isopropylsulfonyl)phenoxy]phenyl}quinazoline;
4-({3-[4-chloro-3-(8-chloroquinazolin-4-yl)phenoxy]phenyl}sulfonyl)butan-1-ol;
4-[(3-{4-chloro-3-[8-(trifluoromethyl)quinazolin-4-yl]phenoxy}phenyl)sulfonyl]butan-1-ol;
3-[(3-{4-chloro-3-[8-(trifluoromethyl)quinazolin-4-yl]phenoxy}phenyl)sulfonyl]propan-1-ol;
4-(2-chloro-5-{[3-(methylsulfonyl)benzyl]oxy}phenyl)-8-(trifluoromethyl)quinazoline;
4-{2-chloro-5-[3-fluoro-5-(methylsulfonyl)phenoxy]phenyl}-8-(trifluoromethyl)quinazoline;
4-{2-chloro-5-[3-(ethylsulfonyl)-5-fluorophenoxy]phenyl}-8-(trifluoromethyl)quinazoline;
4-{2-chloro-5-[3-chloro-5-(methylsulfonyl)phenoxy]phenyl}-8-(trifluoromethyl)quinazoline;
8-chloro-4-(2-chloro-5-{[3-(methylsulfonyl)benzyl]oxy}phenyl)quinazoline;
8-chloro-4-{2-chloro-5-[3-fluoro-5-(methylsulfonyl)phenoxy]phenyl}quinazoline;
8-chloro-4-{2-chloro-5-[3-(ethylsulfonyl)-5-fluorophenoxy]phenyl}quinazoline;
8-chloro-4-{2-chloro-5-[3-chloro-5-(methylsulfonyl)phenoxy]phenyl}quinazoline;
3-[(3-{4-fluoro-3-[8-(trifluoromethyl)quinazolin-4-yl]phenoxy}phenyl)sulfonyl]propan-1-ol;
4-[(3-{4-fluoro-3-[8-(trifluoromethyl)quinazolin-4-yl]phenoxy}phenyl)sulfonyl]butan-1-ol;
4-({3-[3-(8-chloroquinazolin-4-yl)-4-fluorophenoxy]phenyl}sulfonyl)butan-1-ol;
3-[(3-{4-chloro-3-[2-methyl-8-(trifluoromethyl)quinazolin-4-yl]phenoxy}phenyl)sulfonyl]-butan-1-ol; and
4-(2-fluoro-5-{3-[(methylsulfonyl)methyl]phenoxy}phenyl)-8-(trifluoromethyl)quinazoline;
or N-oxide or a pharmaceutically acceptable salt thereof.

89. A pharmaceutical composition comprising a compound of claim 1, or a pharmaceutically acceptable salt thereof, and a pharmaceutically acceptable carrier.

90. A method of preventing or treating a Liver X receptor-mediated disease or disorder, the method comprising administering to a subject in need of such treatment an effective amount of a compound of claim 1 or a pharmaceutically acceptable salt thereof.

91. A method of preventing or treating a cardiovascular disease, the method comprising administering to a subject in need of such treatment an effective amount of a compound of claim 1 or a pharmaceutically acceptable salt thereof.

92. The method of claim 91, wherein the cardiovascular disease is acute coronary syndrome or restenosis.

93. A method of preventing or treating Alzheimer's disease, the method comprising administering to a subject in need of such treatment an effective amount of a compound of claim 1 or a pharmaceutically acceptable salt thereof.

94. A method of preventing or treating type I or type II diabetes, the method comprising administering to a subject in need of such treatment an effective amount of a compound of claim 1 or a pharmaceutically acceptable salt thereof.

95. A method of preventing or treating an inflammatory disease, the method comprising administering to a subject in need of such treatment an effective amount of a compound of claim 1 or a pharmaceutically acceptable salt thereof.

96. The method of claim 95, wherein the inflammatory disease is multiple sclerosis, rheumatoid arthritis, inflammatory bowel disease, Crohn's disease, endometriosis, LPS-induced sepsis, acute contact dermatitis of the ear, or chronic atherosclerotic inflammation of the artery wall.

97. The method of claim 95, wherein the inflammatory disease is rheumatoid arthritis.

98. A method of treating a connective tissue disease, the method comprising administering to a mammal in need thereof an effective amount of a compound of claim 1 or a pharmaceutically acceptable salt thereof.

99. The method of claim 98, wherein the compound of formula (I) inhibits cartilage degradation and induces cartilage regeneration.

100. The method of claim 98, wherein the compound of formula (I) inhibits aggrecanase activity.

101. The method of claim 98, wherein the compound of formula (I) inhibits elaboration of pro-inflammatory cytokines in osteoarthritic lesions.

103. The method of claim 98, wherein the connective tissue disease is osteoarthritis or tendonitis.

103. The method of claim 98, wherein the mammal is a human.

104. A method of treating skin aging, the method comprising administering to a mammal in need thereof an effective amount of a compound of claim 1 or a pharmaceutically acceptable salt thereof.

105. The method of claim 104, wherein the mammal is a human.

106. The method of claim 104, wherein the compound of formula (I) is topically administered.

107. The method of claim 104, wherein the skin aging is derived from chronological aging, photoaging, steroid-induced skin thinning, or a combination thereof.

108. A method of preventing or treating atherosclerosis and/or atherosclerotic lesions, the method comprising administering to a mammal in need thereof an effective amount of a compound of claim 1 or a pharmaceutically acceptable salt thereof.

109. A method of preventing or treating Syndrome X, the method comprising administering to a mammal in need thereof an effective amount of a compound of claim 1 or a pharmaceutically acceptable salt thereof.

110. A method of preventing or treating obesity, the method comprising administering to a mammal in need thereof an effective amount of a compound of claim 1 or a pharmaceutically acceptable salt thereof.

111. A method of preventing or treating one or more lipid disorders selected from the group consisting of dyslipidemia, hyperlipidemia, hypertriglyceridemia, hypercholesterolemia, low HDL and high LDL, the method comprising administering to a mammal in need thereof an effective amount of a compound of claim 1 or a pharmaceutically acceptable salt thereof.

112. A method of increasing serum HDL cholesterol levels in a subject, the method comprising administering to a mammal in need thereof an effective amount of a compound of claim 1 or a pharmaceutically acceptable salt thereof.

113. A method of decreasing serum LDL cholesterol levels in a subject, the method comprising administering to a mammal in need thereof an effective amount of a compound of claim 1 or a pharmaceutically acceptable salt thereof.

114. A method of increasing reverse cholesterol transport in a subject, the method comprising administering to a mammal in need thereof an effective amount of a compound of claim 1 or a pharmaceutically acceptable salt thereof.

115. A method of decreasing cholesterol absorption in a subject, the method comprising administering to a mammal in need thereof an effective amount of a compound of claim 1 or a pharmaceutically acceptable salt thereof.
